(19) United States
(12) Patent Application Publication
(10) Pub. No.: US 2017/0088532 A1
(43) Pub. Date: Mar. 30, 2017

(54) INHIBITORS OF LPXC

(71) Applicant: The Regents of the University of California, Oakland, CA (US)

(72) Inventors: Seth M. Cohen, Carlsbad, CA (US); David T. Puerta, Carlsbad, CA (US); Christian Perez, San Diego, CA (US)

(21) Appl. No.: 15/173,383

(22) Filed: Jun. 3, 2016

Related U.S. Application Data
(63) Continuation of application No. PCT/US2014/068906, filed on Dec. 5, 2014.
(60) Provisional application No. 61/912,087, filed on Dec. 5, 2013.

(51) Int. Cl. C07D 309/40; C07D 309/36

(52) U.S. Cl. CPC C07D 309/40; C07D 309/36

(57) ABSTRACT

Provided herein, inter alia, are compounds and methods for treating infectious diseases. The compounds provided herein are, inter alia, useful for the treatment of bacterial infections. The compounds provided herein are, inter alia, useful inhibitors of metalloproteins, e.g. UDP-3-O—(R-3-hydroxymyristoyl)-N-acetylglucosamine deacetylase (LpxC).

### CROSS-REFERENCES TO RELATED APPLICATIONS

This application is a continuation of International Application No. PCT/US2014/068906, filed Dec. 5, 2014, which claims the benefit of U.S. Provisional Application No. 61/912,087, filed Dec. 5, 2013, which are incorporated herein by reference in their entirety and for all purposes.

### STATEMENT AS TO RIGHTS TO INVENTIONS MADE UNDER FEDERALLY SPONSORED RESEARCH AND DEVELOPMENT

This invention was made with government support under Grant No. R01GM098435 awarded by the National Institutes of Health. The government has certain rights in the invention.

### BACKGROUND OF THE INVENTION

Multi-drug resistant (MDR) and pan-drug resistant (PDR) bacteria pose an ongoing and increasing challenge to the health care system, particularly in the hospital setting. Many of these infections are associated with the devices commonly used in medical procedures, such as catheters and ventilators, and result in central line-associated blood stream infections, catheter-associated urinary tract infections, and ventilator-associated pneumonia. As known in the art, the most common bacteria accounting for drug resistance in the hospital and community are the ‘ESKAPE’ pathogens: Enterococcus faecium, Staphylococcus aureus, Klebsiella pneumoniae, Acinetobacter baumannii, Pseudomonas aeruginosa, and Enterobacter species.

LpxC (UDP-3-O—(R-3-hydroxymyristoyl)-N-acetylglucosamine deacetylase) has emerged as an attractive target for antibiotic development because it catalyzes the committed step of lipid A biosynthesis required for nearly all Gram-negative bacteria. Lipid A is a phospholipid contained within the outer monolayer of the membrane in Gram-negative bacteria. It is the membrane anchor of lipopolysaccharide (LPS) and is essential to protect Gram-negative bacteria against external agents such as antibiotics. Bacteria lacking lipid A are not viable, and mutants with reduced lipid A are hypersensitive to antibiotics. Therefore, an effective inhibitor of LpxC is a viable strategy for treating Gram-negative infections.

LpxC, a zinc-dependent deacetylase, has been the focus of several pharmaceutical drug development programs over the last decade. Potent LpxC inhibitors have been identified that contain a hydroxamic acid group to bind the catalytic zinc ion in the LpxC active site; however, development has been hampered due to narrow spectrum across the Gram-negative family, poor PK/PD properties (i.e. lack of cell permeability, efficient efflux), and toxicity due to off-target effects. Several of these issues are associated with the well-known limitations of hydroxamic acid metal-binding groups (MBG).

Thus, there is a need in the art for novel and effective antibiotics. The application discloses solutions to this and other problems in the art.

### BRIEF SUMMARY OF THE INVENTION

In a first aspect, there is provided compounds useful, inter alia, as antibiotics that inhibit LpxC. The compounds include those having the structure of formula (IA):

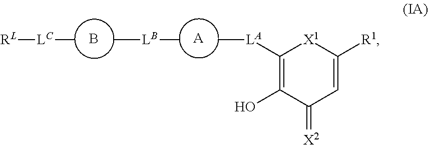

For compounds of formula (IA), R^{L}, L^{C}, ring B, L^{B}, ring A, L^{A}, X¹, R¹ and X² are as defined herein.

In another aspect, there is provided a pharmaceutical composition including a pharmaceutically acceptable excipient and a compound described herein, such as those with structure of formula (IA).

In another aspect, there is provided a method for treating a UDP-3-O—(R-3-hydroxymyristoyl)-N-acetylglucosamine deacetylase (LpxC)-mediated disease in a patient in need of such treatment. The method includes administering a therapeutically effective amount of a compound disclosed herein, such as a compound of formula (IA) or embodiment thereof, to a subject in need thereof.

In another aspect, there is provided a method of treating a UDP-3-O—(R-3-hydroxymyristoyl)-N-acetylglucosamine deacetylase (LpxC)-mediated disease in a patient in need of such treatment. The method includes administering a therapeutically effective amount of a LpxC inhibitor compound, wherein the LpxC inhibitor compound is a substituted or unsubstituted pyridinone, substituted or unsubstituted pyridinethione, substituted or unsubstituted hydroxypyridinone, substituted or unsubstituted hydroxypyridinethione, substituted or unsubstituted hydroxypyranone, substituted or unsubstituted hydroxypyranthione, substituted or unsubstituted pyrocatechol or substituted or unsubstituted pyridinol.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched non-cyclic carbon chain (or carbon), or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable non-cyclic straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′-represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, non-aromatic cyclic versions of “alkyl” and “heteroalkyl,” respectively, wherein the carbons making up the ring or rings do not necessarily need to be bonded to a hydrogen due to all carbon valencies participating in bonds with non-hydrogen atoms. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, 3-hydroxy-cyclobut-3-enyl-1,2, dione, 1H-1,2,4-triazolyl-5(4H)-one, 4H-1,2,4-triazolyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. A heterocycloalkyl moiety may include one ring heteroatom (e.g., O, N, S, Si, or P). A heterocycloalkyl moiety may include two optionally different ring heteroatoms (e.g., O, N, S, Si, or P). A heterocycloalkyl moiety may include three optionally different ring heteroatoms (e.g., O, N, S, Si, or P). A heterocycloalkyl moiety may include four optionally different ring heteroatoms (e.g., O, N, S, Si, or P). A heterocycloalkyl moiety may include five optionally different ring heteroatoms (e.g., O, N, S, Si, or P). A heterocycloalkyl moiety may include up to 8 optionally different ring heteroatoms (e.g., O, N, S, Si, or P).

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. Non-limiting examples of aryl and heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene. A heteroaryl moiety may include one ring heteroatom (e.g., O, N, or S). A heteroaryl moiety may include two optionally different ring heteroatoms (e.g., O, N, or S). A heteroaryl moiety may include three optionally different ring heteroatoms (e.g., O, N, or S). A heteroaryl moiety may include four optionally different ring heteroatoms (e.g., O, N, or S). A heteroaryl moiety may include five optionally different ring heteroatoms (e.g., O, N, or S). An aryl moiety may have a single ring. An aryl moiety may have two optionally different rings. An aryl moiety may have three optionally different rings. An aryl moiety may have four optionally different rings. A heteroaryl moiety may have one ring. A heteroaryl moiety may have two optionally different rings. A heteroaryl moiety may have three optionally different rings. A heteroaryl moiety may have four optionally different rings. A heteroaryl moiety may have five optionally different rings.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,”, “cycloalkyl”, “heterocycloalkyl”, “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂N(R)(′R″—NRSO₂R′), —CN, and —NO₂ in a number ranging from zero to (2m′+1), where m′ is the total number of carbon atoms in such radical. R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, NR″C(O)₂R′, NRC(NR′R″)═NR′″, S(O)R′, —S(O)₂R′, —S(O)₂N(R′)(R″, —NRSO₂R′), —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Where a moiety is substituted with an R substituent, the group may be referred to as “R-substituted.” Where a moiety is R-substituted, the moiety is substituted with at least one R substituent and each R substituent is optionally different. For example, where a moiety herein is R^{1A}-substituted or unsubstituted alkyl, a plurality of R^{1A} substituents may be attached to the alkyl moiety wherein each R^{1A} substituent is optionally different. Where an R-substituted moiety is substituted with a plurality R substituents, each of the R-substituents may be differentiated herein using a prime symbol (′) such as R′, R″, etc. For example, where a moiety is R^{1A}-substituted or unsubstituted alkyl, and the moiety is substituted with a plurality of R^{1A} substituents, the plurality of R^{1A} substitutents may be differentiated as R^{1A}′, R^{1A}″, R^{1A}′″, etc. In some embodiments, the plurality of R substituents is 3. In some embodiments, the plurality of R substituents is 2.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties:
(A) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, heteroaryl, substituted with at least one substituent selected from:
   (i) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, heteroaryl, substituted with at least one substituent selected from:
      (a) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents than are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Provided herein are agents (e.g. compounds, drugs, therapeutic agents) that may be in a prodrug form. Prodrugs of the compounds described herein are those compounds that readily undergo chemical changes under select physiological conditions to provide the final agents (e.g. compounds, drugs, therapeutic agents). Additionally, prodrugs can be converted to agents (e.g. compounds, drugs, therapeutic agents) by chemical or biochemical methods in an ex vivo environment. Prodrugs described herein include compounds that readily undergo chemical changes under select physiological conditions to provide agents (e.g. compounds, drugs, therapeutic agents) to a biological system (e.g. in a subject, in a cancer cell, in the extracellular space near a cancer cell).

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

Descriptions of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease.

An “effective amount” is an amount sufficient to accomplish a stated purpose (e.g. achieve the effect for which it is administered, treat a disease, reduce enzyme activity, increase enzyme activity, reduce protein function, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug or prodrug is an amount of a drug or prodrug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “associated” or “associated with” in the context of a substance or substance activity or function associated with a disease (e.g. infectious disease, hyperproliferative disease, cancer) means that the disease is caused by (in whole or in part), or a symptom of the disease is caused by (in whole or in part) the substance or substance activity or function. As used herein, what is described as being associated with a disease, if a causative agent, could be a target for treatment of the disease. For example, a disease associated with infection may be treated with an agent (e.g. compound as described herein) effective as an antibiotic.

“Control” or “control experiment” or “standard control” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g. chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme. In some embodiments contacting includes allowing a compound described herein to interact with a protein or enzyme.

As defined herein, the term “inhibition”, “inhibit”, “inhibiting” and the like in reference to a protein-inhibitor (e.g. antagonist) interaction means negatively affecting (e.g. decreasing) the level of activity or function of the protein relative to the level of activity or function of the protein in the absence of the inhibitor. In some embodiments inhibition refers to reduction of a disease or symptoms of disease. Thus, inhibition may include, at least in part, partially or totally blocking stimulation, decreasing, preventing, or delaying activation, or inactivating, desensitizing, or down-regulating signal transduction or enzymatic activity or the amount of a protein.

As defined herein, the term “activation”, “activate”, “activating” and the like in reference to a protein-activator (e.g. agonist) interaction means positively affecting (e.g. increasing) the activity or function of the protein relative to the activity or function of the protein in the absence of the activator (e.g. compound described herein). Thus, activation may include, at least in part, partially or totally increasing stimulation, increasing or enabling activation, or activating, sensitizing, or up-regulating signal transduction or enzymatic activity or the amount of a protein decreased in a disease. Activation may include, at least in part, partially or totally increasing stimulation, increasing or enabling activation, or activating, sensitizing, or up-regulating signal transduction or enzymatic activity or the amount of a protein.

“Chemotherapeutic” or “chemotherapeutic agent” is used in accordance with its plain ordinary meaning and refers to a chemical composition or compound having antineoplastic properties or the ability to inhibit the growth or proliferation of cells.

“Patient” or “subject in need thereof” or “subject” refers to a living organism suffering from or prone to a disease or condition that can be treated by administration of a compound or pharmaceutical composition or by a method, as provided herein. Non-limiting examples include humans, other mammals, bovines, rats, mice, dogs, monkeys, goat, sheep, cows, deer, and other non-mammalian animals. In some embodiments, a patient is human. In some embodiments, a subject is human.

“Disease” or “condition” refer to a state of being or health status of a patient or subject capable of being treated with a compound, pharmaceutical composition, or method provided herein. In some embodiments, the disease results from an infection.

As used herein, the term “infectious disease” refers to a disease or condition related to the presence of an organism (the agent or infectious agent) within or contacting the subject or patient. Examples include a bacterium, fungus, virus, or other microorganism. A “bacterial infectious disease” is an infectious disease wherein the organism is a bacterium.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g. anti-cancer agent). The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g. to reduce metabolic degradation, to increase degradation of a prodrug and release of the drug, detectable agent). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the infection causing agent resulting in endocytosis. By using liposomes, particularly where the liposome surface carries receptor ligands specific for target cells, or are otherwise preferentially directed to a specific target, one can focus the delivery of the compositions of the present invention into the target cells in vivo. The compositions of the present invention can also be delivered as nanoparticles.

Pharmaceutical compositions provided by the present invention include compositions wherein the active ingredient (e.g. compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., reducing, eliminating, or slowing the progression of disease symptoms (e.g. symptoms of infection). Determination of a therapeutically effective amount of a compound of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g. symptoms of cancer), kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds described herein can be used in combination with one another, with other active agents known to be useful in treating cancer, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another. In some embodiments, the compounds described herein may be combined with treatments for cancer such as radiation or surgery.

### II. Compound

Compounds provided herein are useful, for example, as inhibitors of metalloproteins such as LpxC (UDP-3-O—(R-3-hydroxymyristoyl)-N-acetylglucosamine deacetylase). LpxC is a Zn-dependent deacetylase essential to the survival of Gram-negative bacteria because LpxC is responsible for the first committed step in LPA (endotoxin) synthesis, a key component of the bacterial membrane.

Inhibitors of metalloproteins, e.g., LpxC, may include two parts: A metal-binding group (MBG) and a “backbone” designed to interact with the protein of interest. The vast majority of metalloprotein inhibitors use a hydroxamic acid moiety as the metal-binding group. Hydroxamic acids have well known chemical and metabolic liabilities limiting their potential in drug applications. In embodiments, compounds disclosed herein are unique, novel and more effective in a clinical setting as they contain non-hydroxamate metal-binding groups which provide a wider chemical space to address the limitations of hydroxamic acids. Additionally, these groups can by modified to improve on potency, selectivity and PK properties.

In one aspect, there is provided a compound with structure of formula (PI):

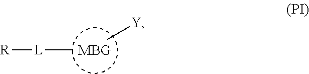

wherein “MBG” (“Metal Binding Group) is a non-hydroxamate metal binding group. Linker L is a substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R and Y are independently hydrogen, halogen, —CN, —SH, —OH, —COOH, —NH₂, —CONH₂, nitro, —CF₃, —CCl₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms “non-hydroxamate” and the like refer, in the usual and customary sense, to a chemical moiety which does not include a hydroxamate moiety. Thus, a “non-hydroxamate metal binding group” is a metal binding group which does not contain a hydroxamate moiety.

In one embodiment, the compound is one or more of the following.

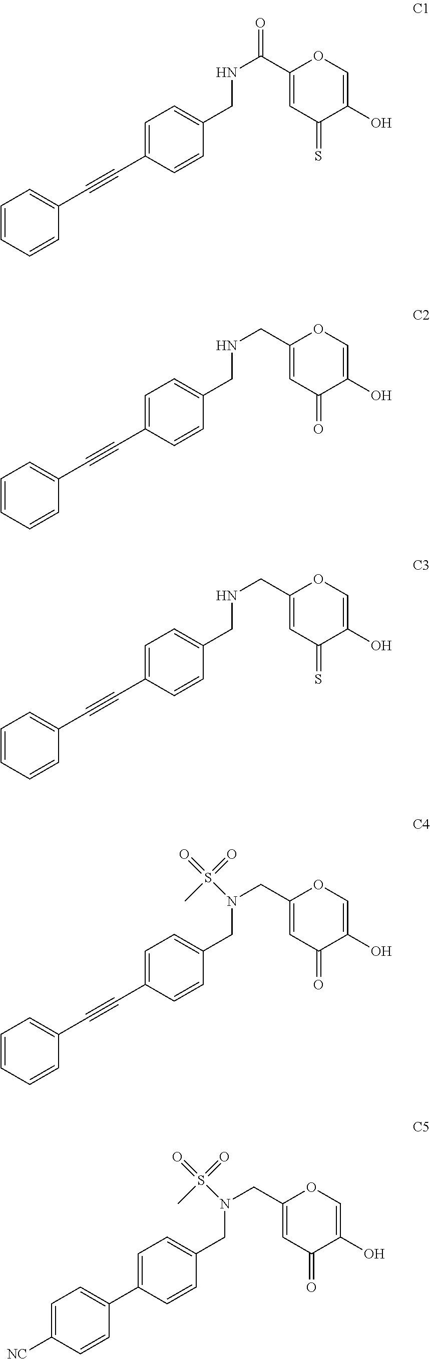 [SMILES: O=C(NCc1ccc(C#Cc2ccccc2)cc1)c1cc(=S)c(O)co1 ; O=c1cc(CNCc2ccc(C#Cc3ccccc3)cc2)occ1O ; Oc1coc(CNCc2ccc(C#Cc3ccccc3)cc2)cc1=S ; CS(=O)(=O)N(Cc1ccc(C#Cc2ccccc2)cc1)Cc1cc(=O)c(O)co1 ; CS(=O)(=O)N(Cc1ccc(-c2ccc(C#N)cc2)cc1)Cc1cc(=O)c(O)co1]

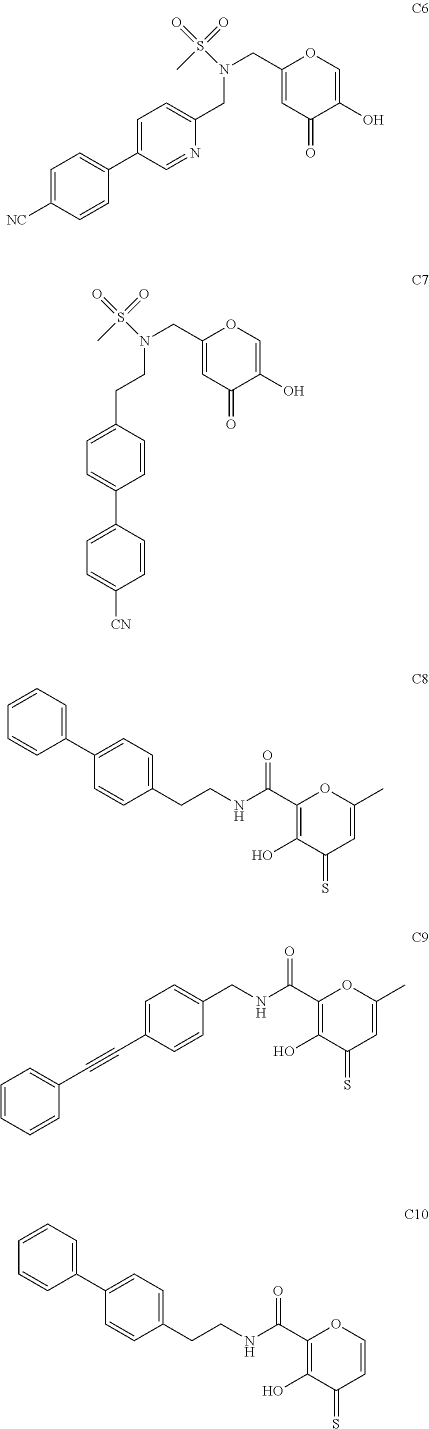 [SMILES: CS(=O)(=O)N(Cc1ccc(-c2ccc(C#N)cc2)cn1)Cc1cc(=O)c(O)co1 ; CS(=O)(=O)N(CCc1ccc(-c2ccc(C#N)cc2)cc1)Cc1cc(=O)c(O)co1 ; Cc1cc(=S)c(O)c(C(=O)NCCc2ccc(-c3ccccc3)cc2)o1 ; Cc1cc(=S)c(O)c(C(=O)NCc2ccc(C#Cc3ccccc3)cc2)o1 ; O=C(NCCc1ccc(-c2ccccc2)cc1)c1occc(=S)c1O]

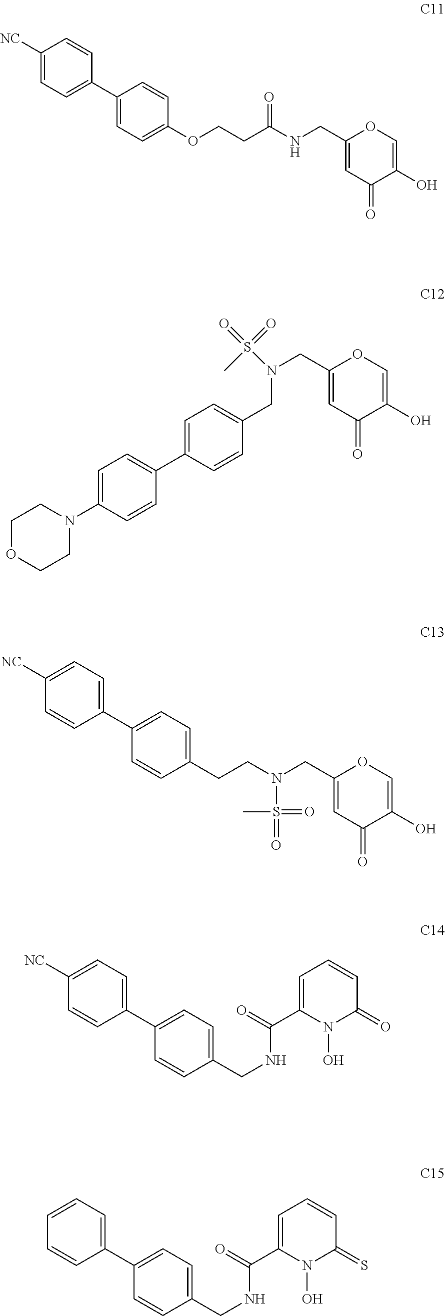 [SMILES: N#Cc1ccc(-c2ccc(OCCC(=O)NCc3cc(=O)c(O)co3)cc2)cc1 ; CS(=O)(=O)N(Cc1ccc(-c2ccc(N3CCOCC3)cc2)cc1)Cc1cc(=O)c(O)co1 ; CS(=O)(=O)N(CCc1ccc(-c2ccc(C#N)cc2)cc1)Cc1cc(=O)c(O)co1 ; N#Cc1ccc(-c2ccc(CNC(=O)c3cccc(=O)n3O)cc2)cc1 ; O=C(NCc1ccc(-c2ccccc2)cc1)c1cccc(=S)n1O]

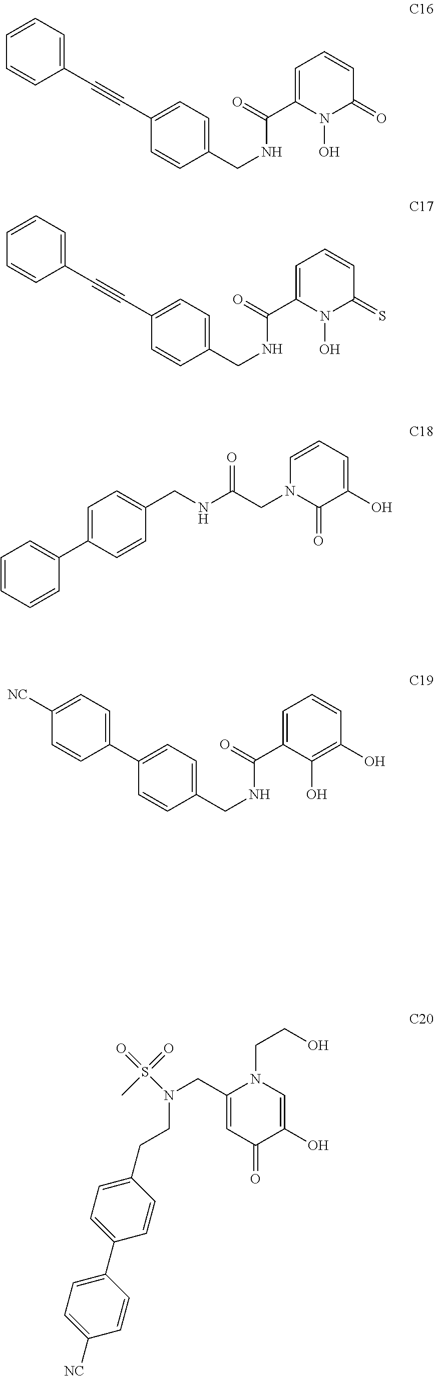 [SMILES: O=C(NCc1ccc(C#Cc2ccccc2)cc1)c1cccc(=O)n1O ; O=C(NCc1ccc(C#Cc2ccccc2)cc1)c1cccc(=S)n1O ; O=C(Cn1cccc(O)c1=O)NCc1ccc(-c2ccccc2)cc1 ; N#Cc1ccc(-c2ccc(CNC(=O)c3cccc(O)c3O)cc2)cc1 ; CS(=O)(=O)N(CCc1ccc(-c2ccc(C#N)cc2)cc1)Cc1cc(=O)c(O)cn1CCO]

In another aspect, there is provided a compound with structure of formula (PII) following:

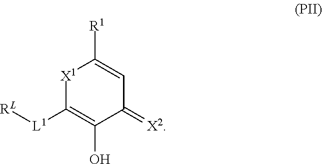

Regarding formula (PII), R¹ is hydrogen, —CN, —SH, —OH, —COOH, —NH₂, —CONH₂, nitro, —CF₃, —CCl₃, substituted or unsubstituted alkyl, or substituted or unsubstituted heteroalkyl. L¹ is substituted or unsubstituted alkylene, or substituted or unsubstituted heteroalkylene. R^{L} is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. X¹ is C, N, O or S. X² is O or S.

In embodiments, R¹ is hydrogen, methyl, hydroxymethyl, —C(O)OCH₃, —CH₂S(O)₂CH₃, or —CH₂S(O)₂-phenyl. L¹ may be —NH—CH₂—. X¹ may be C. X¹ may be N. In one embodiment, X¹ is O. X¹ may also be S. X² may be O. In embodiments, X² is S.

In embodiments, the compound is one or more of the following.

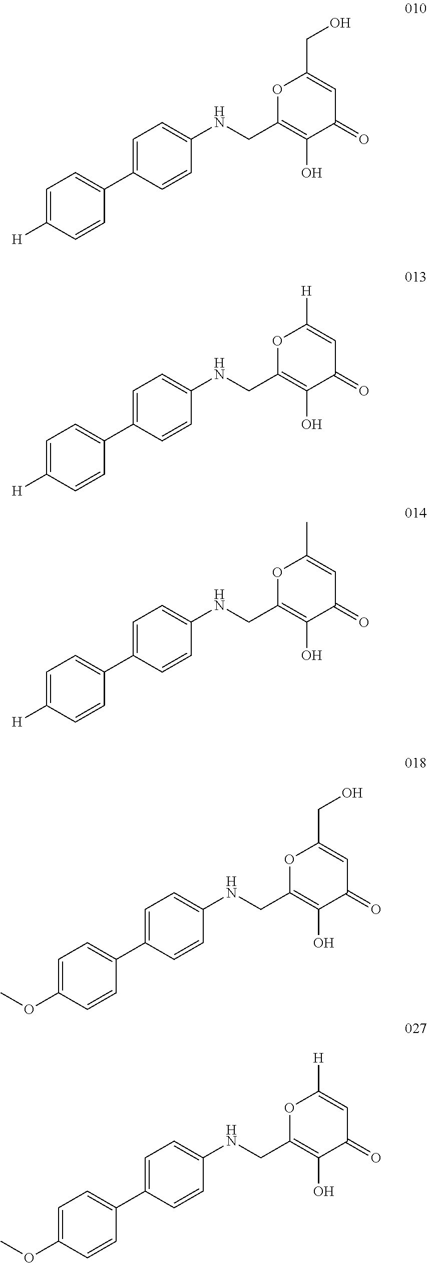 [SMILES: O=c1cc(CO)oc(CNc2ccc(-c3ccccc3)cc2)c1O ; O=c1ccoc(CNc2ccc(-c3ccccc3)cc2)c1O ; Cc1cc(=O)c(O)c(CNc2ccc(-c3ccccc3)cc2)o1 ; COc1ccc(-c2ccc(NCc3oc(CO)cc(=O)c3O)cc2)cc1 ; COc1ccc(-c2ccc(NCc3occc(=O)c3O)cc2)cc1]

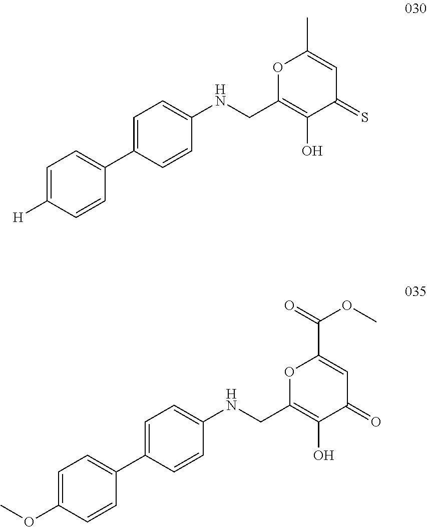

In embodiments, the compound has a structure as set forth following.

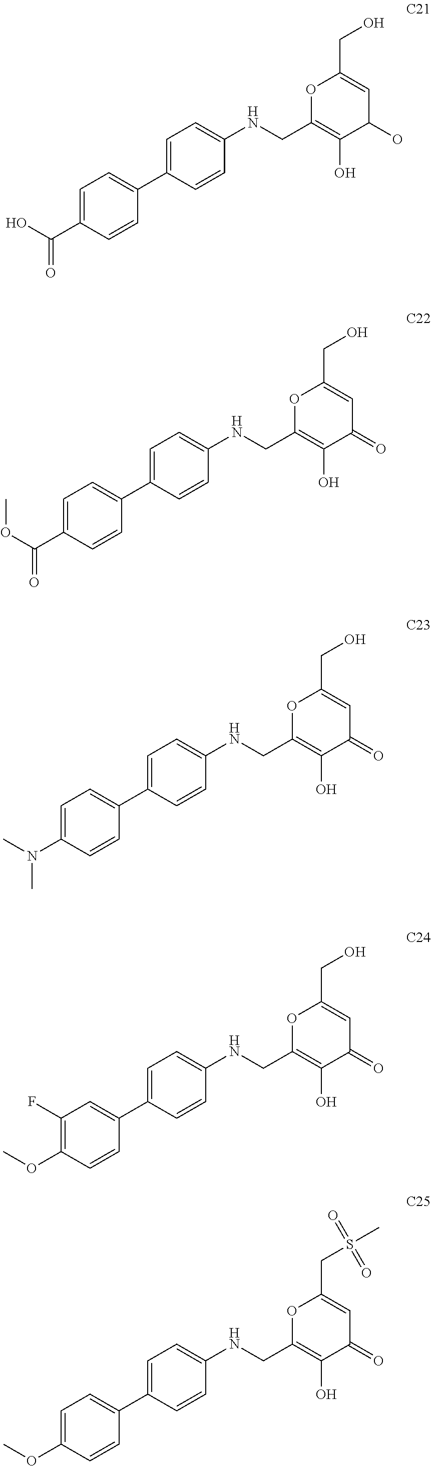

In embodiments, the compound is one or more of the following.

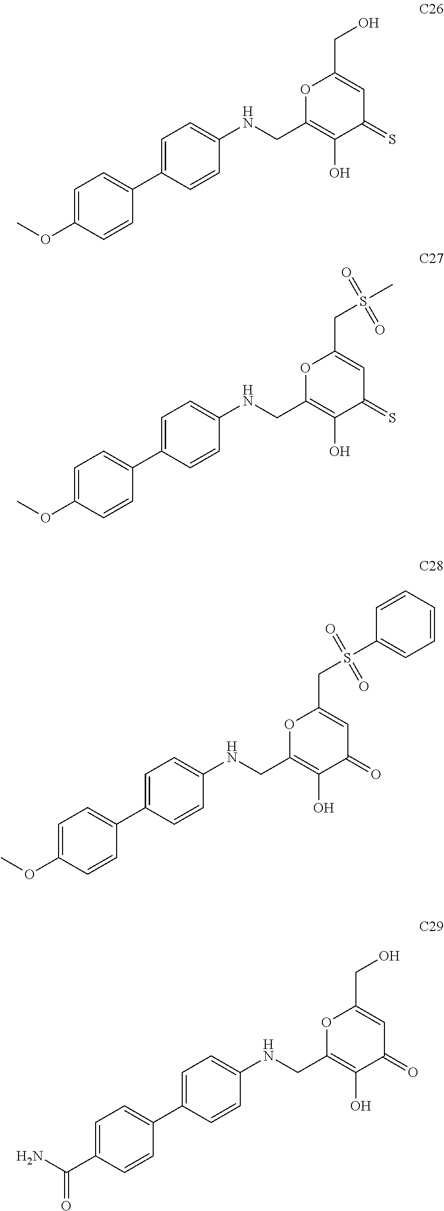

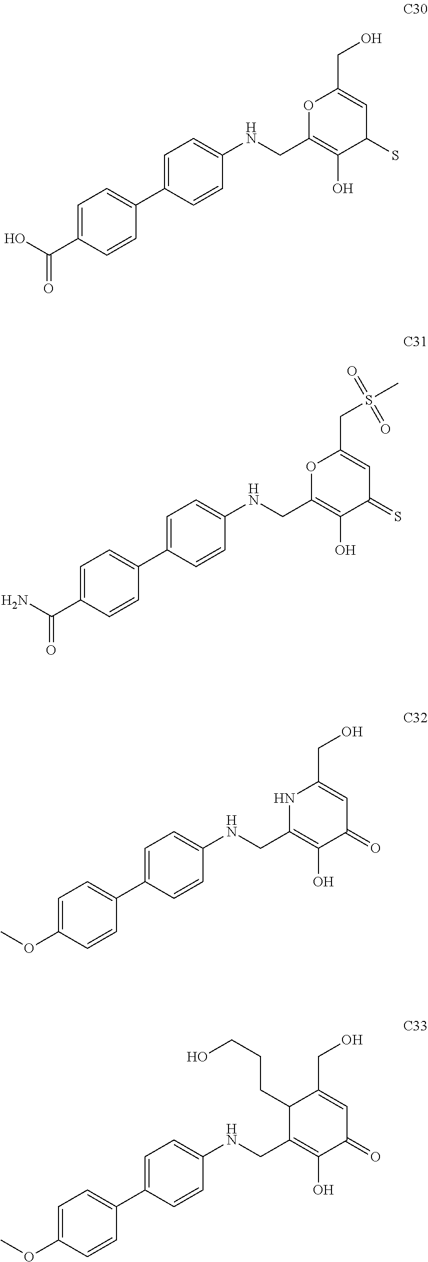

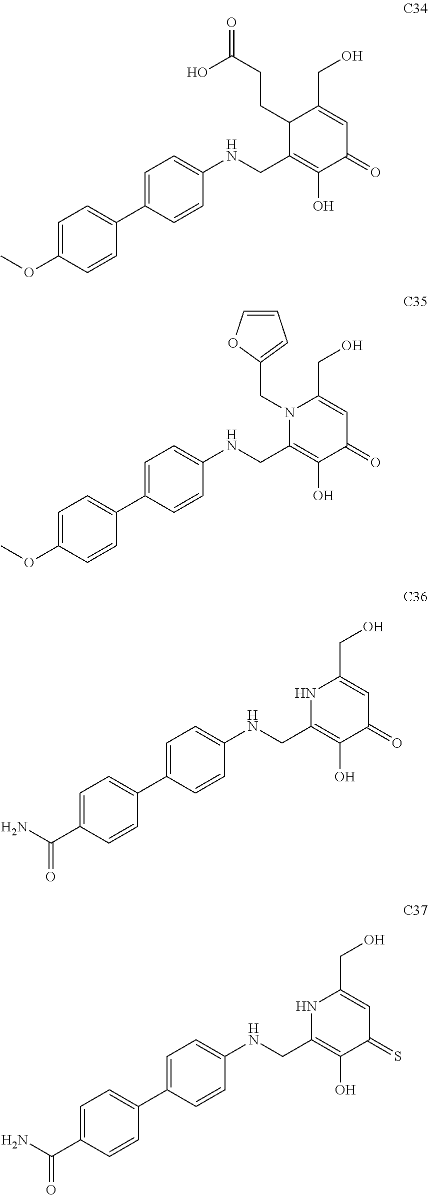 [SMILES: COc1ccc(-c2ccc(NCC3=C(O)C(=O)C=C(CO)C3CCC(=O)O)cc2)cc1 ; COc1ccc(-c2ccc(NCc3c(O)c(=O)cc(CO)n3Cc3ccco3)cc2)cc1 ; NC(=O)c1ccc(-c2ccc(NCc3[nH]c(CO)cc(=O)c3O)cc2)cc1 ; NC(=O)c1ccc(-c2ccc(NCc3[nH]c(CO)cc(=S)c3O)cc2)cc1]

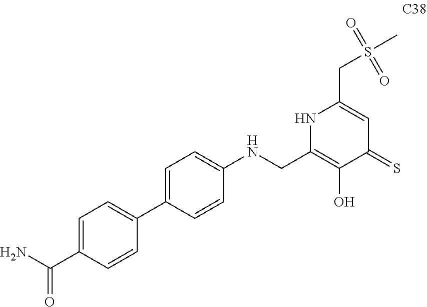 [SMILES: CS(=O)(=O)Cc1cc(=S)c(O)c(CNc2ccc(-c3ccc(C(N)=O)cc3)cc2)[nH]1]

In another aspect, there is provided a compound with structure of formula (PIII):

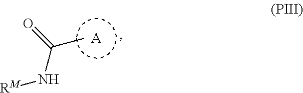

wherein ring “A” is a substituted or unsubstituted six-membered cycloalkyl, substituted or unsubstituted six-membered heterocycloalkyl, or substituted or unsubstituted six-membered heteroaryl. R^{M} is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, the compound is one or more of the following:

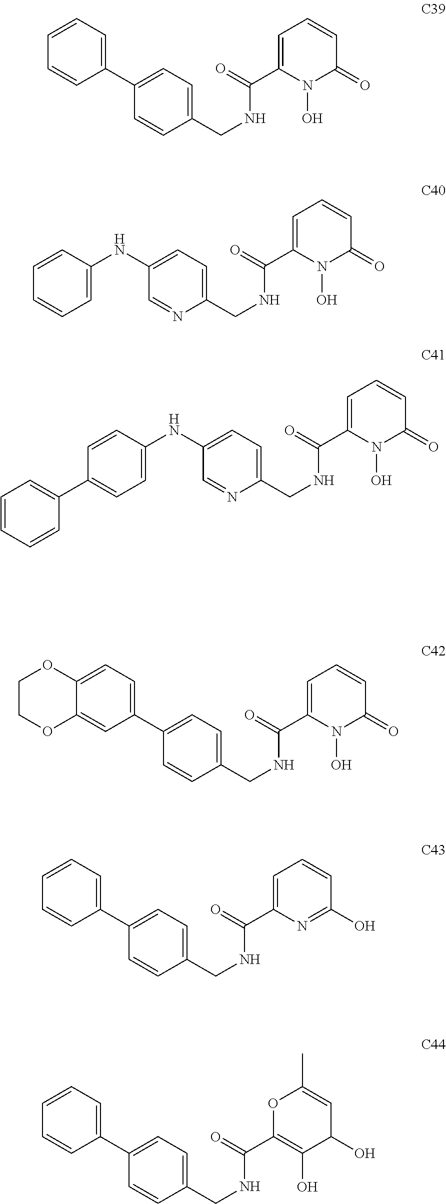 [SMILES: O=C(NCc1ccc(-c2ccccc2)cc1)c1cccc(=O)n1O ; O=C(NCc1ccc(Nc2ccccc2)cn1)c1cccc(=O)n1O ; O=C(NCc1ccc(Nc2ccc(-c3ccccc3)cc2)cn1)c1cccc(=O)n1O ; O=C(NCc1ccc(-c2ccc3c(c2)OCCO3)cc1)c1cccc(=O)n1O ; O=C(NCc1ccc(-c2ccccc2)cc1)c1cccc(O)n1 ; CC1=CC(O)C(O)=C(C(=O)NCc2ccc(-c3ccccc3)cc2)O1]

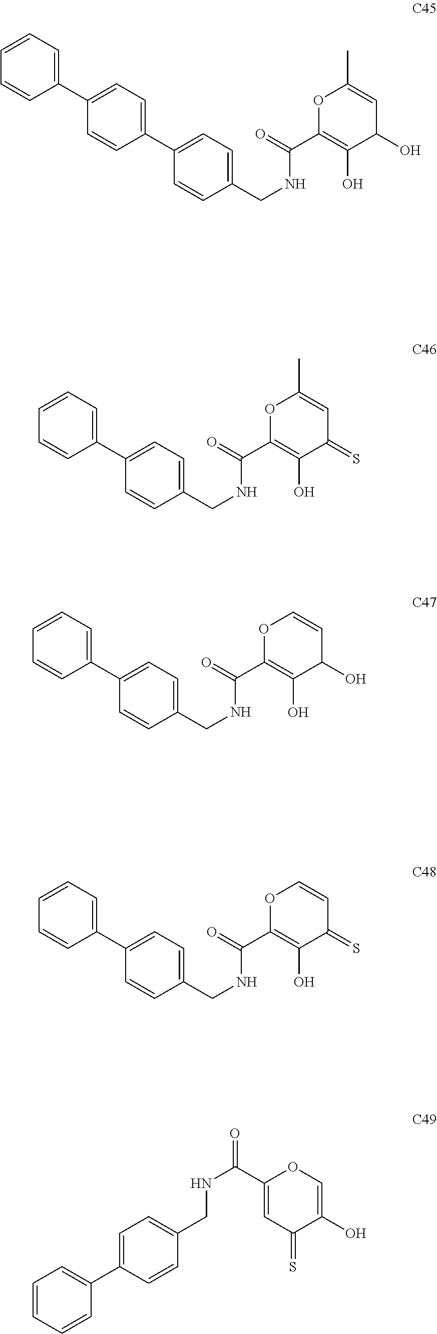 [SMILES: CC1=CC(O)C(O)=C(C(=O)NCc2ccc(-c3ccc(-c4ccccc4)cc3)cc2)O1 ; Cc1cc(=S)c(O)c(C(=O)NCc2ccc(-c3ccccc3)cc2)o1 ; O=C(NCc1ccc(-c2ccccc2)cc1)C1=C(O)C(O)C=CO1 ; O=C(NCc1ccc(-c2ccccc2)cc1)c1occc(=S)c1O ; O=C(NCc1ccc(-c2ccccc2)cc1)c1cc(=S)c(O)co1]

In embodiments, the compound has the formula:

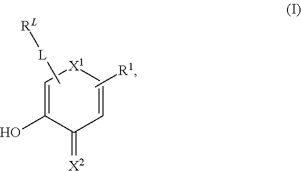

Regarding formula (I), R¹ is independently hydrogen, halogen, —CX^{a}₃, —CN, —SR³, —SO₂Cl, —SOₙ₁R³, —SOᵥ₁NR³R⁴, —NHNH₂, —ONR³R⁴, —NHC═(O)NHNH₂, —NHC═(O)NR³R⁴, —N(O)ₘ₁, —NR³R⁴, —NH—O—R³, —C(O)R³, —C(O)—OR³, —C(O)NR³R⁴, —OR³, -L⁴-SOₙ₁R³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{L} is independently hydrogen, halogen, —CX^{b}₃, —CN, —SR⁵, —SO₂Cl, —SOₙ₂R⁵, —SOᵥ₂NR⁵R⁶, —NHNH₂, —ONR⁵R⁶, —NHC═(O)NHNH₂, —NHC═(O)NR⁵R⁶, —N(O)ₘ₂, —NR⁵R⁶, —NH—O—R⁵, —C(O)R⁵, —C(O)—OR⁵, —C(O)NR⁵R⁶, —OR⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³, R⁴, R⁵, R⁶ and R⁷ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L is independently a bond, —S(O)—, —S(O)₂NH—, —NHS(O)₂—, —C(O)O—, —OC(O)—, —C(O)—, —C(O)NH—, —C(O)NH-L⁵-, -L⁵-C(O)NH—, —NH—, —NHC(O)—, —NH-L⁵-, -L⁵-NH—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L⁵ is independently —C(O)—, —C(O)—NH₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

X^{a} and X^{b} are independently —F, —Cl, —Br, or —I. n₁ and n₂ are independently an integer from 0 to 4. m₁ and m₂ are independently an integer from 1 to 2. v₁ and v₂ are independently an integer from 1 to 2. X¹ is C, N, O, NR⁷ or S. X² is O or S.

In embodiments, R⁷ is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{7A}-substituted or unsubstituted alkyl, R^{7A}-substituted or unsubstituted heteroalkyl, R^{7A}-substituted or unsubstituted cycloalkyl, R^{7A} substituted or unsubstituted heterocycloalkyl, R^{7A}-substituted or unsubstituted aryl, or R^{7A}-substituted or unsubstituted heteroaryl. R⁷ may be R^{7A}-substituted or unsubstituted (e.g., C₁-C₂₀ or C₁-C₆) alkyl, R^{7A}-substituted or unsubstituted (e.g., 2 to 20 membered or 2 to 6 membered) heteroalkyl, R^{7A}-substituted or unsubstituted (e.g., C₃-C₈ or C₅-C₇) cycloalkyl, R^{7A}-substituted or unsubstituted (e.g., 3 to 8 membered or 3 to 6 membered) heterocycloalkyl, R^{7A-}substituted or unsubstituted (e.g., C₅-C₁₀ or C₅-C₆) aryl, or R^{7A}-substituted or unsubstituted (e.g., 5 to 10 membered or 5 to 6 membered) heteroaryl.

R^{7A} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{7B}-substituted or unsubstituted alkyl, R^{7B}-substituted or unsubstituted heteroalkyl, R^{B}-substituted or unsubstituted cycloalkyl, R^{7B}-substituted or unsubstituted heterocycloalkyl, R^{B}-substituted or unsubstituted aryl, or R^{7B}-substituted or unsubstituted heteroaryl. R^{7A} may be R^{7B}-substituted or unsubstituted (e.g., C₁-C₂₀ or C₁-C₆) alkyl, R^{7B}-substituted or unsubstituted (e.g., 2 to 20 membered or 2 to 6 membered) heteroalkyl, R^{B}-substituted or unsubstituted (e.g., C₃-C₈ or C₅-C₇) cycloalkyl, R^{7B}-substituted or unsubstituted (e.g., 3 to 8 membered or 3 to 6 membered) heterocycloalkyl, R^{7B}-substituted or unsubstituted (e.g., C₅-C₁₀ or C₅-C₆) aryl, or R^{7B}-substituted or unsubstituted (e.g., 5 to 10 membered or 5 to 6 membered) heteroaryl.

R^{7B} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{7C}-substituted or unsubstituted alkyl, R^{7C}-substituted or unsubstituted heteroalkyl, R^{7C}-substituted or unsubstituted cycloalkyl, R^{7C}-substituted or unsubstituted heterocycloalkyl, R^{7C}-substituted or unsubstituted aryl, or R^{7C}-substituted or unsubstituted heteroaryl. R^{7B} may be R^{7C}-substituted or unsubstituted (e.g., C₁-C₂₀ or C₁-C₆) alkyl, R^{7C}-substituted or unsubstituted (e.g., 2 to 20 membered or 2 to 6 membered) heteroalkyl, R^{7C}-substituted or unsubstituted (e.g., C₃-C₈ or C₅-C₇) cycloalkyl, R^{7C-}substituted or unsubstituted (e.g., 3 to 8 membered or 3 to 6 membered) heterocycloalkyl, R^{7C}-substituted or unsubstituted (e.g., C₅-C₁₀ or C₅-C₆) aryl, or R^{7C}-substituted or unsubstituted (e.g., 5 to 10 membered or 5 to 6 membered) heteroaryl.

R^{7C} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. R^{7C} may be unsubstituted (e.g., C₁-C₂₀ or C₁-C₆) alkyl, unsubstituted (e.g., 2 to 20 membered or 2 to 6 membered) heteroalkyl, unsubstituted (e.g., C₃-C₈ or C₅-C₇) cycloalkyl, unsubstituted (e.g., 3 to 8 membered or 3 to 6 membered) heterocycloalkyl, unsubstituted (e.g., C₅-C₁₀ or C₅-C₆) aryl, or unsubstituted (e.g., 5 to 10 membered or 5 to 6 membered) heteroaryl.

In embodiments, L is

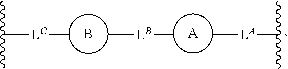

wherein L^{A} is independently a bond, —S(O)—, —S(O)₂NH—, —NHS(O)₂—, —C(O)O—, —OC(O)—, —C(O)—, —C(O)NH—, —C(O)NH-L⁴-, —NH—, —NHC(O)—, —NH-L⁴-, -L⁴-NH—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylen. L^{B} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L^{C} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. L⁴ is independently —C(O)—, —C(O)—NH₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. Ring A is substituted or unsubstituted cycloalkylene, substituted or unsubstituted heterocycloalkylene, substituted or unsubstituted arylene, or substituted or unsubstituted heteroarylene. Ring B is substituted or unsubstituted cycloalkylene, substituted or unsubstituted heterocycloalkylene, substituted or unsubstituted arylene, or substituted or unsubstituted heteroarylene.

In embodiments, the compound has the formula

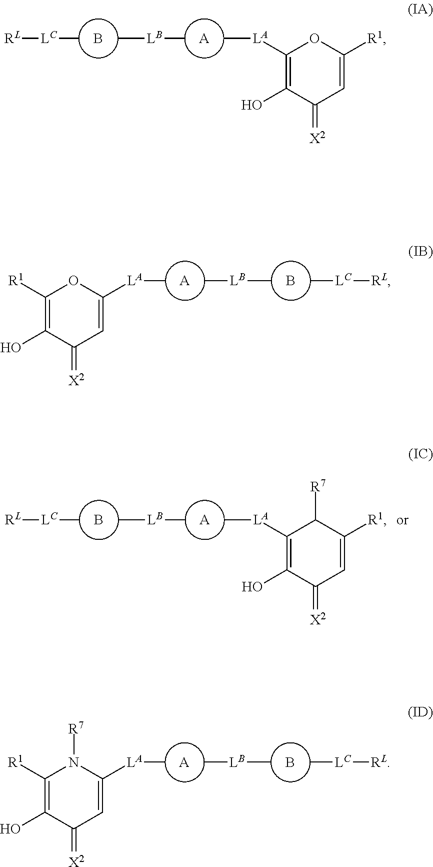

In embodiments, the compound has the formula:

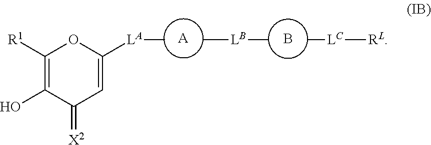

In embodiments, R¹ is hydrogen.

In embodiments, R^{L} is independently hydrogen, —CN or substituted or unsubstituted heterocycloalkyl. In embodiments, R^{L} is substituted or unsubstituted 5-6 membered heterocycloalkyl. In embodiments, R^{L} is unsubstituted 6 membered heterocycloalkyl. In embodiments, R^{L} is unsubstituted morpholinyl.

In embodiments, L^{A} is independently —C(O)NH-L⁴-, -L⁴-NH— or substituted or unsubstituted heteroalkylene. In embodiments, L^{A} is —C(O)NH-L⁴- and L⁴ is substituted or unsubstituted C₁-C₁₀ alkylene. In embodiments, L⁴ is substituted or unsubstituted C₁-C₅ alkylene. In embodiments, L⁴ is unsubstituted C₁-C₅ alkylene. In embodiments, L⁴ is unsubstituted C₁-C₃ alkylene. In embodiments, L⁴ is methylene.

In embodiments, L^{A} is -L⁴-NH— and L⁴ is substituted or unsubstituted C₁-C₁₀ alkylene. In embodiments, L⁴ is substituted or unsubstituted C₁-C₅ alkylene. In embodiments, L⁴ is unsubstituted C₁-C₅ alkylene. In embodiments, L⁴ is methylene.

In embodiments, L^{A} is substituted or unsubstituted 2 to 8 membered heteroalkylene. In embodiments, L^{A} is substituted or unsubstituted 3 to 6 membered heteroalkylene.

In embodiments, L^{A} is set forth herein for a compound of formula (IA) and embodiments thereof.

In embodiments, L^{B} is independently a bond or unsubstituted C₁-C₁₀ alkylene. In embodiments, L^{B} is a bond. In embodiments, L^{B} is unsubstituted C₁-C₅ alkylene. In embodiments, L^{B} is unsaturated unsubstituted C₁-C₅ alkylene. In embodiments, L^{B} is unsaturated unsubstituted C₁-C₃ alkylene. In embodiments, L^{B} is —C≡C—.

In embodiments, L^{C} is a bond.

In embodiments, ring A is substituted or unsubstituted C₅-C₆ arylene. In embodiments, ring A is substituted or unsubstituted C₆ arylene. In embodiments, ring A is unsubstituted phenylene.

In embodiments, ring B is substituted or unsubstituted C₅-C₆ arylene. In embodiments, ring B is unsubstituted phenylene.

In embodiments, the compound has the structure of formula (IC):

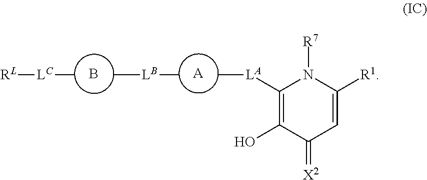

In embodiments of formula (IC) or other compounds provided herein, R¹ is R^{1A} substituted or unsubstituted C₁-C₁₀ alkyl, wherein R^{1A} is —OH or —SO₂CH₃. In embodiments, R¹ is R^{1A}-substituted C₁-C₅ alkyl. In embodiments, R¹ is R^{1A}-substituted C₁-C₃ alkyl. In embodiments, R¹ is R^{1A}-substituted C₁ alkyl.

In embodiments, R^{L} is —C(O)NR⁵R⁶ or —OR⁵. In embodiments, R^{L} is —C(O)NR⁵R⁶ and R⁵ and R⁶ are independently hydrogen or substituted or unsubstituted C₁-C₅ alkyl. In embodiments, R^{L} is —C(O)NR⁵R⁶ and R⁵ and R⁶ are hydrogen. In embodiments, R^{L} is —OR⁵ and R⁵ is substituted or unsubstituted C₁-C₅ alkyl. In embodiments, R⁵ is unsubstituted C₁-C₅ alkyl. In embodiments, R⁵ is methyl.

In embodiments, L^{A} is -L⁴-NH— and L⁴ is substituted or unsubstituted C₁-C₁₀ alkylene. In embodiments, L⁴ is unsubstituted C₁-C₅ alkylene. In embodiments, L⁴ is methylene.

In embodiments, ring A is substituted or unsubstituted C₅-C₆ arylene. In embodiments, ring A is unsubstituted phenylene.

In embodiments, ring B is substituted or unsubstituted C₅-C₆ arylene. In embodiments, ring B is unsubstituted phenylene.

In embodiments, R⁷ is independently hydrogen or R^{7A}-substituted or unsubstituted alkyl and R^{7A} is —OH, —COOH or unsubstituted heteroaryl. In embodiments, R⁷ is R^{7A-}substituted C₁-C₁₀ alkyl. In embodiments, R⁷ is R^{7A}-substituted C₁-C₅ alkyl. In embodiments, R⁷ is R^{7A}-substituted C₃ alkyl and R^{7A} is —OH. In embodiments, R⁷ is R^{7A}-substituted C₂ alkyl and R^{7A} is —COOH. In embodiments, R⁷ is R^{7A}-substituted C₁ alkyl and R^{7A} is unsubstituted 5-membered heteroaryl. In embodiments, R^{7A} is furanyl.

In embodiments, the compound has the structure of formula (ID):

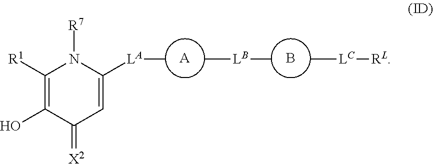

In embodiments of formula (ID) or other compounds provided herein, R¹ is hydrogen. In embodiments, R^{L} is —CN.

In embodiments, L^{A} is substituted or unsubstituted 2-8 membered heteroalkylene. In embodiments, L^{A} is substituted 4 membered heteroalkylene.

In embodiments, ring A is substituted or unsubstituted C₅-C₆ arylene. In embodiments, ring A is unsubstituted phenylene.

In embodiments, ring B is substituted or unsubstituted C₅-C₆ arylene. In embodiments, ring B is unsubstituted phenylene.

In embodiments, R⁷ is independently hydrogen or R^{7A}-substituted or unsubstituted alkyl and R^{7A} is —OH. In embodiments, R⁷ is R^{7A}-substituted C₁-C₅ alkyl. In embodiments, R⁷ is R^{7A}-substituted C₂ alkyl.

In embodiments, the compound has the structure of formula (II):

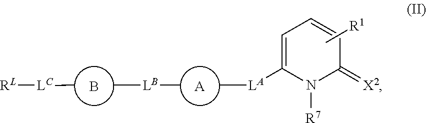

In embodiments of formula (II) or other compounds provided herein, R¹ is independently hydrogen, halogen, —CX^{a}₃, —CN, —SR³, —SO₂Cl, —SOₙ₁R³, —SOᵥ₁NR³R⁴, —NHNH₂, —ONR³R⁴, —NHC═(O)NHNH₂, —NHC═(O)NR³R⁴, —N(O)ₘ₁, —NR³R⁴, —NH—O—R³, —C(O)R³, —C(O)—OR³, —C(O)NR³R⁴, —OR³, -L⁴-SOₙR³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{L} is independently hydrogen, halogen, —CX^{b}₃, —CN, —SR⁵, —SO₂Cl, —SOₙ₂R⁵, —SOᵥ₂NR⁵R⁶, —NHNH₂, —ONR⁵R⁶, —NHC═(O)NHNH₂, —NHC═(O)NR⁵R⁶, —N(O)ₘ₂, —NR⁵R⁶, —NH—O—R⁵, —C(O)R⁵, —C(O)—OR⁵, —C(O)NR⁵R⁶, —OR⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R³, R⁴, R⁵, R⁶ and R⁷ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. L^{A} is independently a bond, —S(O)—, —S(O)₂NH—, —NHS(O)₂—, —C(O)O—, —OC(O)—, —C(O)—, —C(O)NH—, —C(O)NH-L⁴-, —NH—, —NHC(O)—, —NH-L⁴-, -L⁴-NH—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. L^{B} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. L^{C} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. L⁴ is independently —C(O)—, —C(O)—NH₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. Ring A is substituted or unsubstituted cycloalkylene, substituted or unsubstituted heterocycloalkylene, substituted or unsubstituted arylene, or substituted or unsubstituted heteroarylene. Ring B is substituted or unsubstituted cycloalkylene, substituted or unsubstituted heterocycloalkylene, substituted or unsubstituted arylene, or substituted or unsubstituted heteroarylene. X^{a} and X^{b} are independently —F, —Cl, —Br, or —I. n₁ and n₂ are independently an integer from 0 to 4. m₁ and m₂ are independently an integer from 1 to 2. v₁ and v₂ are independently an integer from 1 to 2. X² is O or S.

In embodiments, R¹ is hydrogen.

In embodiments, R^{L} is independently hydrogen, —CN, substituted or unsubstituted heterocycloalkyl or substituted or unsubstituted aryl. In embodiments, R^{L} is hydrogen. In embodiments, R^{L} is —CN. In embodiments, R^{L} is substituted or unsubstituted 5-6 membered heterocycloalkyl. In embodiments, R^{L} is unsubstituted 6 membered heterocycloalkyl. In embodiments, R^{L} is dioxanyl. In embodiments, R^{L} is substituted or unsubstituted 5-6 membered aryl. In embodiments, R^{L} is unsubstituted 5-6 membered aryl. In embodiments, R^{L} is unsubstituted phenyl.

In embodiments, L^{A} is —C(O)NH-L⁴- and L⁴ is substituted or unsubstituted C₁-C₁₀ alkylene. In embodiments, L⁴ is substituted or unsubstituted C₁-C₅ alkylene. In embodiments, L⁴ is unsubstituted C₁-C₃ alkylene. In embodiments, L⁴ is methylene.

In embodiments, L^{B} is a bond, substituted or unsubstituted alkylene or —NH—. In embodiments, L^{B} is a bond. In embodiments, L^{B} is substituted or unsubstituted C₁-C₅ alkylene. In embodiments, L^{B} is unsubstituted C₁-C₅ alkylene. In embodiments, L^{B} is unsaturated unsubstituted C₁-C₅ alkylene. In embodiments, L^{B} is acetylene. In embodiments, L^{B} is —NH—.

In embodiments, L^{C} is a bond.

In embodiments, ring A is substituted or unsubstituted C₅-C₆ arylene or substituted or unsubstituted 5-6 membered heteroarylene. In embodiments, ring A is unsubstituted phenylene. In embodiments, ring A is unsubstituted 6 membered heteroarylene. In embodiments, ring A is unsubstituted pyridinylene.

In embodiments, ring B is substituted or unsubstituted C₅-C₆ arylene. In embodiments, ring B is unsubstituted phenylene.

In embodiments, R⁷ is —OH.

In embodiments, the compound has the formula

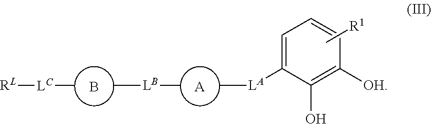

In embodiments of formula (III) of other compounds provided herein, R¹ is independently hydrogen, halogen, —CX^{a}₃, —CN, —SR³, —SO₂Cl, —SOᵥ₁R³, —SOᵥ₁NR³R⁴, —NHNH₂, —ONR³R⁴, —NHC═(O)NHNH₂, —NHC═(O)NR³R⁴, —N(O)ₘ₁, —NR³R⁴, —NH—O—R³, —C(O)R³, —C(O)—OR³, —C(O)NR³R⁴, —OR³, -L⁴-SOₙ₁R³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{L} is independently hydrogen, halogen, —CX^{b}₃, —CN, —SR⁵, —SO₂Cl, —SOₙ₂R⁵, —SOᵥ₂NR⁵R⁶, —NHNH₂, —ONR⁵R⁶, —NHC═(O)NHNH₂, —NHC═(O)NR⁵R⁶, —N(O)ₘ₂, —NR⁵R⁶, —NH—O—R, —C(O)R⁵, —C(O)—OR⁵, —C(O)NR⁵R⁶, —OR⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R³, R⁴, R⁵ and R⁶ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. L^{A} is independently a bond, —S(O)—, —S(O)₂NH—, —NHS(O)₂—, —C(O)O—, —OC(O)—, —C(O)—, —C(O)NH—, —C(O)NH-L⁴-, —NH—, —NHC(O)—, —NH-L⁴-, -L⁴-NH—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. L^{B} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. L^{C} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. L⁴ is independently —C(O)—, —C(O)—NH₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. Ring A is substituted or unsubstituted cycloalkylene, substituted or unsubstituted heterocycloalkylene, substituted or unsubstituted arylene, or substituted or unsubstituted heteroarylene. Ring B is substituted or unsubstituted cycloalkylene, substituted or unsubstituted heterocycloalkylene, substituted or unsubstituted arylene, or substituted or unsubstituted heteroarylene. X^{a} and X^{b} are independently —F, —Cl, —Br, or —I. n₁ and n₂ are independently an integer from 0 to 4. m₁ and m₂ are independently an integer from 1 to 2. v₁ and v₂ are independently an integer from 1 to 2.

In embodiments, R¹ is hydrogen. In embodiments, R^{L} is —CN.

In embodiments, L^{A} is —C(O)NH-L⁴- and L⁴ is substituted or unsubstituted C₁-C₁₀ alkylene.

In embodiments, L⁴ is substituted or unsubstituted C₁-C₅ alkylene. In embodiments, L⁴ is unsubstituted C₁-C₃ alkylene. In embodiments, L⁴ is methylene.

In embodiments, L^{B} is a bond.

In embodiments, L^{C} is a bond.

In embodiments, ring A is substituted or unsubstituted C₅-C₆ arylene. In embodiments, ring A is unsubstituted phenylene.

In embodiments, ring B is substituted or unsubstituted C₅-C₆ arylene. In embodiments, ring B is unsubstituted phenylene.

In embodiments, the compound has the formula

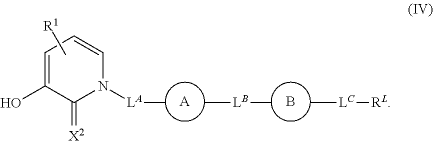

In embodiments of formula (IV) or other compounds provided herein, R¹ is independently hydrogen, halogen, —CX^{a}₃, —CN, —SR³, —SO₂Cl, —SOₙ₁R³, —SOᵥ₁NR³R⁴, —NHNH₂, —ONR³R⁴, —NHC═(O)NHNH₂, —NHC═(O)NR³R⁴, —N(O)ₘ₁, —NR³R⁴, —NH—O—R³, —C(O)R³, —C(O)—OR³, —C(O)NR³R⁴, —OR³, -L⁴-SOₙ₁R³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{L} is independently hydrogen, halogen, —CX^{b}₃, —CN, —SR⁵, —SO₂Cl, —SOₙ₂R⁵, —SOᵥ₂NR⁵R⁶, —NHNH₂, —ONR⁵R⁶, —NHC═(O)NHNH₂, —NHC═(O)NR⁵R⁶, —N(O)ₘ₂, —NR⁵R⁶, —NH—O—R⁵, —C(O)R⁵, —C(O)—OR⁵, —C(O)NR⁵R⁶, —OR′, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R³, R⁴, R⁵ and R⁶ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. L^{A} is independently a bond, —S(O)—, —S(O)₂NH—, —NHS(O)₂—, —C(O)O—, —OC(O)—, —C(O)—, —C(O)NH—, —C(O)NH-L⁴-, —NH—, —NHC(O)—, —NH-L⁴-, -L⁴-NH—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. L^{B} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. L^{C} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. L⁴ is independently —C(O)—, —C(O)—NH₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. Ring A is substituted or unsubstituted cycloalkylene, substituted or unsubstituted heterocycloalkylene, substituted or unsubstituted arylene, or substituted or unsubstituted heteroarylene. Ring B is substituted or unsubstituted cycloalkylene, substituted or unsubstituted heterocycloalkylene, substituted or unsubstituted arylene, or substituted or unsubstituted heteroarylene. X^{a} and X^{b} are independently —F, —Cl, —Br, or —I. n₁ and n₂ are independently an integer from 0 to 4. m₁ and m₂ are independently an integer from 1 to 2. v₁ and v₂ are independently an integer from 1 to 2. X² is O or S.

In embodiments, R¹ is hydrogen.

In embodiments, R^{L} is hydrogen.

In embodiments, L^{A} is substituted or unsubstituted hetero alkylene. In embodiments, L^{A} is substituted 2-8 membered heteroalkylene. In embodiments, L^{A} is substituted 4 membered heteroalkylene.

In embodiments, L^{B} is a bond.

In embodiments, L^{C} is a bond.

In embodiments, ring A is substituted or unsubstituted C₅-C₆ arylene. In embodiments, ring A is unsubstituted phenylene.

In embodiments, ring B is substituted or unsubstituted C₅-C₆ arylene. In embodiments, ring B is unsubstituted phenylene.

In another embodiment, the compound has the formula:

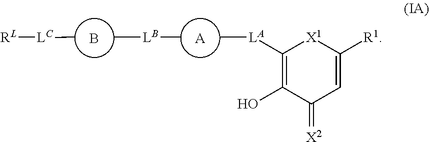

In embodiments of formula (IA) or other compounds provided herein, R¹ is independently hydrogen, halogen, —CX^{a}₃, —CN, —SR³, —SO₂Cl, —SOₙ₁R³, —SOᵥ₁NR³R⁴, —NHNH₂, —ONR³R⁴, —NHC═(O)NHNH₂, —NHC═(O)NR³R⁴, —N(O)ₘ₁, —NR³R⁴, —NH—O—R³, —C(O)R³, —C(O)—OR³, —C(O)NR³R⁴, —OR³, -L⁴-SOₙ₁R³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{L} is independently hydrogen, halogen, —CX^{b}₃, —CN, —SR⁵, —SO₂Cl, —SOₙ₂R⁵, —SOᵥ₂NR⁵R⁶, —NHNH₂, —ONR⁵R⁶, —NHC═(O)NHNH₂, —NHC═(O)NR⁵R⁶, —N(O)ₘ₂, —NR⁵R⁶, —NH—O—R⁵, —C(O)R⁵, —C(O)—OR⁵, —C(O)NR⁵R⁶, —OR⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³, R⁴, R⁵, R⁶ and R⁷ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L^{A} is independently a bond, —S(O)—, —S(O)₂NH—, —NHS(O)₂—, —C(O)O—, —OC(O)—, —C(O)—, —C(O)NH—, —C(O)NH-L⁴-, -L⁴-C(O)NH—, —NH—, —NHC(O)—, —NH-L⁴-, -L⁴-NH—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L^{B} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L^{C} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L⁴ is independently —C(O)—, —C(O)—NH—, —NH—C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Ring A is substituted or unsubstituted cycloalkylene, substituted or unsubstituted heterocycloalkylene, substituted or unsubstituted arylene, or substituted or unsubstituted heteroarylene.

Ring B is substituted or unsubstituted cycloalkylene, substituted or unsubstituted heterocycloalkylene, substituted or unsubstituted arylene, or substituted or unsubstituted heteroarylene.

X¹ is C, N, O, NR⁷ or S. X² is independently O or S. X^{a} and X^{b} are independently F, Cl, Br, or I. n₁ and n₂ are independently an integer from 0 to 4. m₁ and m₂ are independently an integer from 1 to 2. v₁ and v₂ are independently an integer from 1 to 2.

In embodiments, if L^{A} is —C(O)NH-L⁴, L⁴ is methylene, ring A and ring B are each unsubstituted phenyl, L^{C} is a bond or —O—, and X¹ is O, then R^{L} is not hydrogen.

In embodiments, L^{A} is independently a bond, —S(O)—, —S(O)₂NH—, —NHS(O)₂—, —C(O)O—, —OC(O)—, —C(O)—, -L⁴-C(O)NH—, —NH—, —NHC(O)—, —NH-L⁴-, -L⁴-NH—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, if L^{A} is -L⁴-C(O)NH—, then L⁴ is not a bond.

In embodiments, R¹ is independently R^{1A}-substituted or unsubstituted alkyl, R^{1A-}substituted or unsubstituted heteroalkyl, R^{1A}-substituted or unsubstituted cycloalkyl, R^{1A} substituted or unsubstituted heterocycloalkyl, R^{1A}-substituted or unsubstituted aryl, or R^{1A} substituted or unsubstituted heteroaryl. R^{1A} may be independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1B}-substituted or unsubstituted (e.g., C₁-C₂₀ or C₁-C₆) alkyl, R^{1B}-substituted or unsubstituted (e.g., 2 to 20 membered or 2 to 6 membered) heteroalkyl, R^{1B}-substituted or unsubstituted (e.g., C₃-C₈ or C₅-C₇) cycloalkyl, R^{1B}-substituted or unsubstituted (e.g., 3 to 8 membered or 3 to 6 membered) heterocycloalkyl, R^{1B} substituted or unsubstituted (e.g., C₅-C₁₀ or C₅-C₆) aryl, or R^{1B}-substituted or unsubstituted (e.g., 5 to 10 membered or 5 to 6 membered) heteroaryl.

R^{1B} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1C}-substituted or unsubstituted (e.g., C₁-C₂₀ or C₁-C₆) alkyl, R^{1C}-substituted or unsubstituted (e.g., 2 to 20 membered or 2 to 6 membered) heteroalkyl, R^{1C}-substituted or unsubstituted (e.g., C₃-C₈ or C₅-C₇) cycloalkyl, R^{1C}-substituted or unsubstituted (e.g., 3 to 8 membered or 3 to 6 membered) heterocycloalkyl, R^{1C}-substituted or unsubstituted (e.g., C₅-C₁₀ or C₅-C₆) aryl, or R^{1C}-substituted or unsubstituted (e.g., 5 to 10 membered or 5 to 6 membered) heteroaryl.

R^{1C} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted (e.g., C₁-C₂₀ or C₁-C₆) alkyl, unsubstituted (e.g., 2 to 20 membered or 2 to 6 membered) heteroalkyl, unsubstituted (e.g., C₃-C₈ or C₅-C₇) cycloalkyl, unsubstituted (e.g., 3 to 8 membered or 3 to 6 membered) heterocycloalkyl, unsubstituted (e.g., C₅-C₁₀ or C₅-C₆) aryl, or unsubstituted (e.g., 5 to 10 membered or 5 to 6 membered) heteroaryl.

In embodiments, R^{L} is independently R^{LA}-substituted or unsubstituted alkyl, R^{LA-}substituted or unsubstituted heteroalkyl, R^{LA}-substituted or unsubstituted cycloalkyl, R^{LA} substituted or unsubstituted heterocycloalkyl, R^{LA}-substituted or unsubstituted aryl, or R^{LA}-substituted or unsubstituted heteroaryl.

R^{LA} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{LA}-substituted or unsubstituted (e.g., C₁-C₂₀ or C₁-C₆) alkyl, R^{LA}-substituted or unsubstituted (e.g., 2 to 20 membered or 2 to 6 membered) heteroalkyl, R^{LA}-substituted or unsubstituted (e.g., C₃-C₈ or C₅-C₇) cycloalkyl, R^{LA}-substituted or unsubstituted (e.g., 3 to 8 membered or 3 to 6 membered) heterocycloalkyl, R^{LA}-substituted or unsubstituted (e.g., C₅-C₁₀ or C₅-C₆) aryl, or R^{LA}-substituted or unsubstituted (e.g., 5 to 10 membered or 5 to 6 membered) heteroaryl.

R^{LB} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{LC}-substituted or unsubstituted (e.g., C₁-C₂₀ or C₁-C₆) alkyl, R^{LC}-substituted or unsubstituted (e.g., 2 to 20 membered or 2 to 6 membered) heteroalkyl, R^{LC-}substituted or unsubstituted (e.g., C₃-C₈ or C₅-C₇) cycloalkyl, R^{LC}-substituted or unsubstituted (e.g., 3 to 8 membered or 3 to 6 membered) heterocycloalkyl, R^{LC}-substituted or unsubstituted (e.g., C₅-C₁₀ or C₅-C₆) aryl, or R^{LC}-substituted or unsubstituted (e.g., 5 to 10 membered or 5 to 6 membered) heteroaryl.

R^{LC} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted (e.g., C₁-C₂₀ or C₁-C₆) alkyl, unsubstituted (e.g., 2 to 20 membered or 2 to 6 membered) heteroalkyl, unsubstituted (e.g., C₃-C₈ or C₅-C₇) cycloalkyl, unsubstituted (e.g., 3 to 8 membered or 3 to 6 membered) heterocycloalkyl, unsubstituted (e.g., C₅-C₁₀ or C₅-C₆) aryl, or unsubstituted (e.g., 5 to 10 membered or 5 to 6 membered) heteroaryl.

In embodiments, R¹ is independently hydrogen, —C(O)—OR³, -L⁴-SOₙ₁R³ or substituted or unsubstituted alkyl. In embodiments, R¹ is hydrogen. In embodiments, R¹ is —C(O)—OR³ and R³ is substituted or unsubstituted C₁-C₁₀ alkyl.

In embodiments, R³ is substituted or unsubstituted C₁-C₅ alkyl. In embodiments, R³ is unsubstituted C₁-C₅ alkyl. In embodiments, R³ is methyl.

In embodiments, R¹ is -L⁴-SOₙ₁R³, R³ is substituted or unsubstituted alkyl or substituted or unsubstituted aryl, and L⁴ is substituted or unsubstituted alkylene. In embodiments, R³ is substituted or unsubstituted C₁-C₁₀ alkyl or substituted or unsubstituted C₅-C₆ aryl. In embodiments, R³ is substituted or unsubstituted C₁-C₅ alkyl. In embodiments, R³ is unsubstituted C₁-C₅ alkyl. In embodiments, R³ is methyl. In embodiments, R³ is unsubstituted C₅-C₆ aryl. In embodiments, R³ is unsubstituted phenyl.

In embodiments, L⁴ is substituted or unsubstituted C₁-C₁₀ alkylene. In embodiments, L⁴ is substituted or unsubstituted C₁-C₅ alkylene. In embodiments, L⁴ is unsubstituted C₁-C₅ alkylene. In embodiments, L⁴ is methylene.

In embodiments, R¹ is substituted or unsubstituted C₁-C₁₀ alkyl. In embodiments, R¹ is substituted or unsubstituted C₁-C₅ alkyl. In embodiments, R¹ unsubstituted C₁-C₅ alkyl. In embodiments, R¹ is methyl.

In embodiments, R¹ is R^{1A}-substituted C₁-C₅ alkyl. In embodiments, R¹ is R^{1A}-substituted C₁ alkyl and R^{1A} is —OH.

In embodiments R^{L} is independently hydrogen, halogen, —OR⁵, —C(O)—OR⁵, —NR⁵R⁶, —C(O)NR⁵R⁶, substituted or unsubstituted heterocycloalkyl or substituted or unsubstituted aryl. In embodiments, R^{L} is hydrogen. In embodiments, R^{L} is halogen. In embodiments, R^{L} is —F.

In embodiments, R^{L} is —OR⁵ and R⁵ is substituted or unsubstituted C₁-C₁₀ alkyl. In embodiments, R⁵ is substituted or unsubstituted C₁-C₅ alkyl. In embodiments, R⁵ is unsubstituted C₁-C₅ alkyl. In embodiments, R⁵ is methyl. In embodiments, R^{L} is —C(O)—OR⁵ and R⁵ is hydrogen or substituted or unsubstituted C₁-C₁₀ alkyl. In embodiments, R⁵ is hydrogen. In embodiments, R⁵ is substituted or unsubstituted C₁-C₅ alkyl. In embodiments, R⁵ is unsubstituted C₁-C₅ alkyl. In embodiments, R⁵ is methyl.

In embodiments, R^{L} is —NR⁵R⁶ and R⁵ and R⁶ are independently hydrogen or substituted or unsubstituted C₁-C₁₀ alkyl. In embodiments, R⁵ and R⁶ are independently hydrogen or substituted or unsubstituted C₁-C₅ alkyl. In embodiments, R⁵ and R⁶ are independently unsubstituted C₁-C₅ alkyl. In embodiments, R⁵ and R⁶ are methyl.

In embodiments, R^{L} is —C(O)NR⁵R⁶ and R⁵ and R⁶ are independently hydrogen or substituted or unsubstituted C₁-C₁₀ alkyl. In embodiments, R⁵ and R⁶ are independently hydrogen or substituted or unsubstituted C₁-C₅ alkyl. In embodiments, R⁵ and R⁶ are independently hydrogen or unsubstituted C₁-C₅ alkyl. In embodiments, R⁵ and R⁶ are independently hydrogen or methyl. In embodiments, R⁵ and R⁶ are hydrogen.

In embodiments, R^{L} is substituted or unsubstituted 5-6 membered heterocycloalkyl. In embodiments, R^{L} is unsubstituted 6 membered heterocycloalkyl. In embodiments, R^{L} is unsubstituted morpholinyl.

In embodiments, R^{L} is substituted or unsubstituted C₅-C₆ aryl. In embodiments, R^{L} is substituted or unsubstituted C₆ aryl. In embodiments, R^{L} is unsubstituted phenyl.

In embodiments, L^{A} is —C(O)NH-L⁴- or -L⁴-NH—. In embodiments, L^{A} is —C(O)NH-L⁴- and L⁴ is substituted or unsubstituted C₁-C₁₀ alkylene. In embodiments, L⁴ is substituted or unsubstituted C₁-C₅ alkylene. In embodiments, L⁴ is unsubstituted C₁-C₅ alkylene. In embodiments, L⁴ is unsubstituted C₁-C₃ alkylene. In embodiments, L⁴ is methylene.

In embodiments, L^{A} is -L⁴-NH— and L⁴ is substituted or unsubstituted C₁-C₁₀ alkylene. In embodiments, L⁴ is substituted or unsubstituted C₁-C₅ alkylene. In embodiments, L⁴ is unsubstituted C₁-C₅ alkylene. In embodiments, L⁴ is unsubstituted C₁-C₃ alkylene. In embodiments, L⁴ is methylene.

In embodiments, if L^{A} is -L⁴-C(O)NH—, then L⁴ is not a bond.

In embodiments, L^{A} is independently a bond, —S(O)—, —S(O)₂NH—, —NHS(O)₂—, —C(O)O—, —OC(O)—, —C(O)—, —C(O)NH—, —C(O)NH-L⁴-, -L⁴-C(O)NH—, —NH—, —NHC(O)—, —NH-L⁴-, -L⁴-NH—, —O—, —S—, R^{LA}-substituted or unsubstituted alkylene, R^{LA1-}substituted or unsubstituted heteroalkylene, R^{LA1}-substituted or unsubstituted cycloalkylene, R^{LA1}-substituted or unsubstituted heterocycloalkylene, R^{LA1}-substituted or unsubstituted arylene, or R^{LA1}-substituted or unsubstituted heteroarylene.

In embodiments, L^{A} is R^{LA1}-substituted or unsubstituted (e.g., C₁-C₂₀ or C₁-C₆) alkylene, R^{LA1}-substituted or unsubstituted (e.g., 2 to 20 membered or 2 to 6 membered) heteroalkylene, R^{LA1}-substituted or unsubstituted (e.g., C₃-C₈ or C₅-C₇) cycloalkylene, R^{LA1-}substituted or unsubstituted (e.g., 3 to 8 membered or 3 to 6 membered) heterocycloalkylene, R^{LA1}-substituted or unsubstituted (e.g., C₅-C₁₀ or C₅-C₆) arylene, or R^{LA1}-substituted or unsubstituted (e.g., 5 to 10 membered or 5 to 6 membered) heteroarylene. In embodiments, L^{A} is R^{LA}-substituted (e.g., C₁-C₂₀ or C₁-C₆) alkylene, R^{LA1}-substituted (e.g., 2 to 20 membered or 2 to 6 membered) heteroalkylene, R^{LA1}-substituted (e.g., C₃-C₈ or C₅-C₇) cycloalkylene, R^{LA1}-substituted (e.g., 3 to 8 membered or 3 to 6 membered) heterocycloalkylene, R^{LA-}substituted (e.g., C₅-C₁₀ or C₅-C₆) arylene, or R^{LA1}-substituted (e.g., 5 to 10 membered or 5 to 6 membered) heteroarylene. In embodiments, L^{A} is unsubstituted (e.g., C₁-C₂₀ or C₁-C₆) alkylene, unsubstituted (e.g., 2 to 20 membered or 2 to 6 membered) heteroalkylene, unsubstituted (e.g., C₃-C₈ or C₅-C₇) cycloalkylene, unsubstituted (e.g., 3 to 8 membered or 3 to 6 membered) heterocycloalkylene, unsubstituted (e.g., C₅-C₁₀ or C₅-C₆) arylene, or unsubstituted (e.g., 5 to 10 membered or 5 to 6 membered) heteroarylene.

R^{LA1} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{LA2}-substituted or unsubstituted (e.g., C₁-C₂₀ or C₁-C₆) alkyl, R^{LA2}-substituted or unsubstituted (e.g., 2 to 20 membered or 2 to 6 membered) heteroalkyl, R^{LA2}-substituted or unsubstituted (e.g., C₃-C₈ or C₅-C₇) cycloalkyl, R^{LA2-}substituted or unsubstituted (e.g., 3 to 8 membered or 3 to 6 membered) heterocycloalkyl, R^{L2}-substituted or unsubstituted (e.g., C₅-C₁₀ or C₅-C₆) aryl, or R^{L2}-substituted or unsubstituted (e.g., 5 to 10 membered or 5 to 6 membered) heteroaryl.

In embodiments, R^{LA1} is R^{LA2}-substituted (e.g., C₁-C₂₀ or C₁-C₆) alkyl, R^{LA2}-substituted (e.g., 2 to 20 membered or 2 to 6 membered) heteroalkyl, R^{LA2}-substituted (e.g., C₃-C₈ or C₅-C₇) cycloalkyl, R^{LA2}-substituted (e.g., 3 to 8 membered or 3 to 6 membered) heterocycloalkyl, R^{LA2}-substituted (e.g., C₅-C₁₀ or C₅-C₆) aryl, or R^{LA2}-substituted (e.g., 5 to 10 membered or 5 to 6 membered) heteroaryl. In embodiments, R^{LA1} is unsubstituted (e.g., C₁-C₂₀ or C₁-C₆) alkyl, unsubstituted (e.g., 2 to 20 membered or 2 to 6 membered) heteroalkyl, unsubstituted (e.g., C₃-C₈ or C₅-C₇) cycloalkyl, unsubstituted (e.g., 3 to 8 membered or 3 to 6 membered) heterocycloalkyl, unsubstituted (e.g., C₅-C₁₀ or C₅-C₆) aryl, or unsubstituted (e.g., 5 to 10 membered or 5 to 6 membered) heteroaryl.

R^{LA2} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted (e.g., C₁-C₂₀ or C₁-C₆) alkyl, unsubstituted (e.g., 2 to 20 membered or 2 to 6 membered) heteroalkyl, unsubstituted (e.g., C₃-C₈ or C₅-C₇) cycloalkyl, unsubstituted (e.g., 3 to 8 membered or 3 to 6 membered) heterocycloalkyl, unsubstituted (e.g., C₅-C₁₀ or C₅-C₆) aryl, or unsubstituted (e.g., 5 to 10 membered or 5 to 6 membered) heteroaryl.

In embodiments, L^{B} is independently a bond or unsubstituted C₁-C₁₀ alkylene. In embodiments, L^{B} is a bond. In embodiments, L^{B} is unsubstituted C₁-C₅ alkylene. In embodiments, L^{B} is unsaturated unsubstituted C₁-C₅ alkylene. In embodiments, L^{B} is unsaturated unsubstituted C₁-C₃ alkylene. In embodiments, L^{B} is —C≡C—. In embodiments, L^{B} is unsaturated unsubstituted C₁-C₄ alkylene. In embodiments, L^{B} is —C≡C—C≡C—.

In embodiments, L^{C} is a bond.

In embodiments, ring A is substituted or unsubstituted C₅-C₆ arylene. In embodiments, ring A is substituted or unsubstituted C₆ arylene. In embodiments, ring A is unsubstituted phenylene.

In embodiments, ring B is is substituted or unsubstituted C₅-C₆ arylene. In embodiments, ring B is substituted or unsubstituted C₆ arylene. In embodiments, ring B is unsubstituted phenylene.

In embodiments, the compound is one or more of the following:

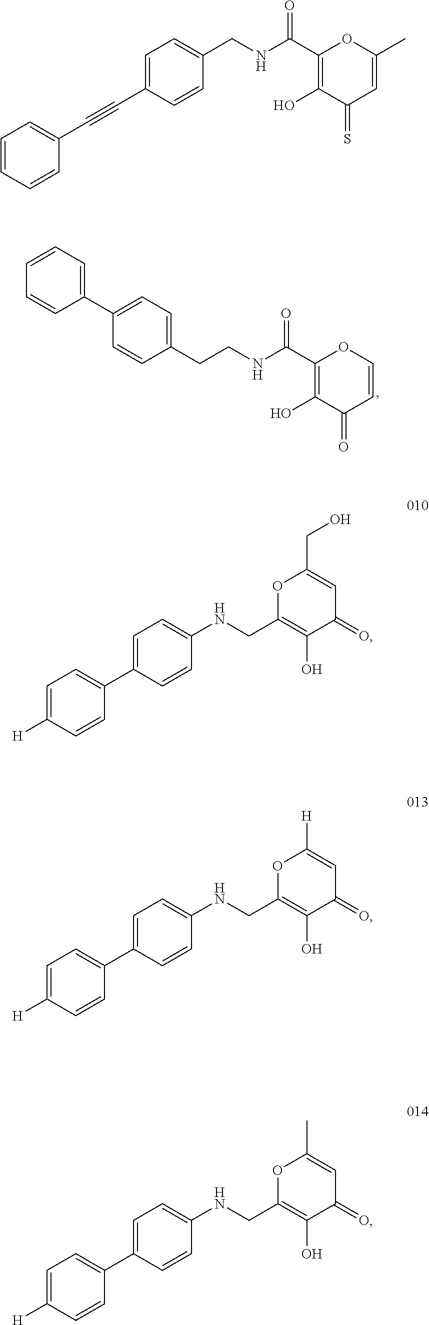 [SMILES: Cc1cc(=S)c(O)c(C(=O)NCc2ccc(C#Cc3ccccc3)cc2)o1 ; O=C(NCCc1ccc(-c2ccccc2)cc1)c1occc(=O)c1O ; O=c1cc(CO)oc(CNc2ccc(-c3ccccc3)cc2)c1O ; O=c1ccoc(CNc2ccc(-c3ccccc3)cc2)c1O ; Cc1cc(=O)c(O)c(CNc2ccc(-c3ccccc3)cc2)o1]

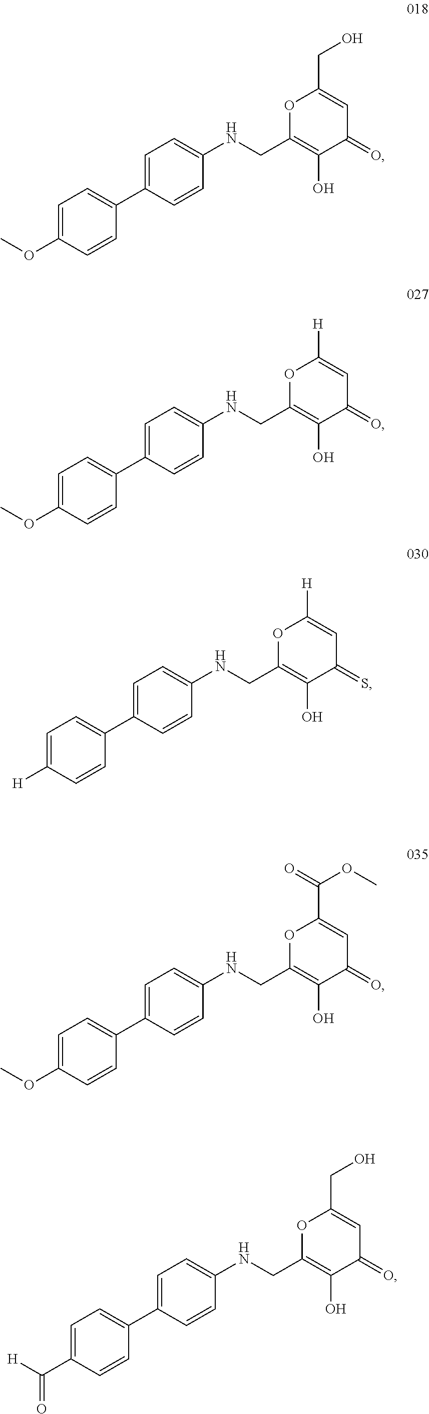

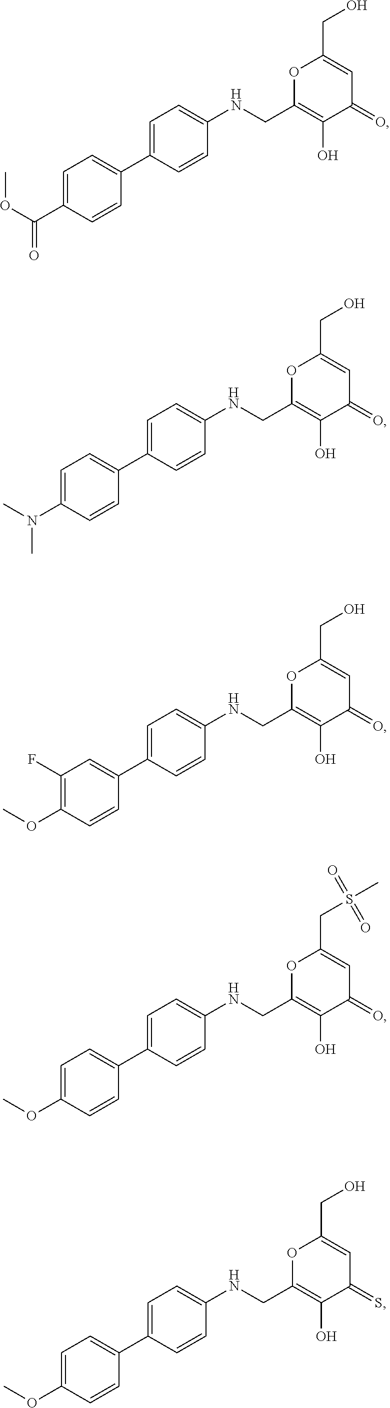

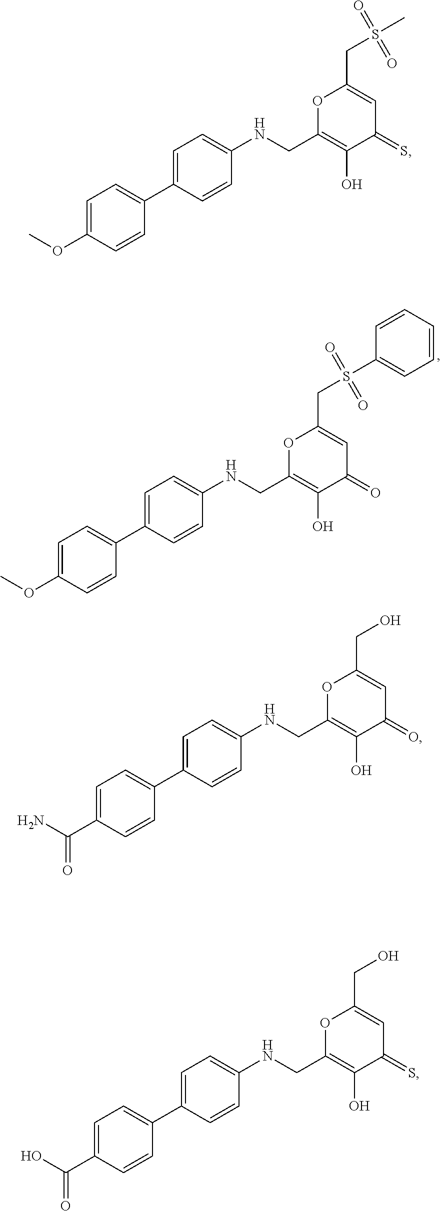

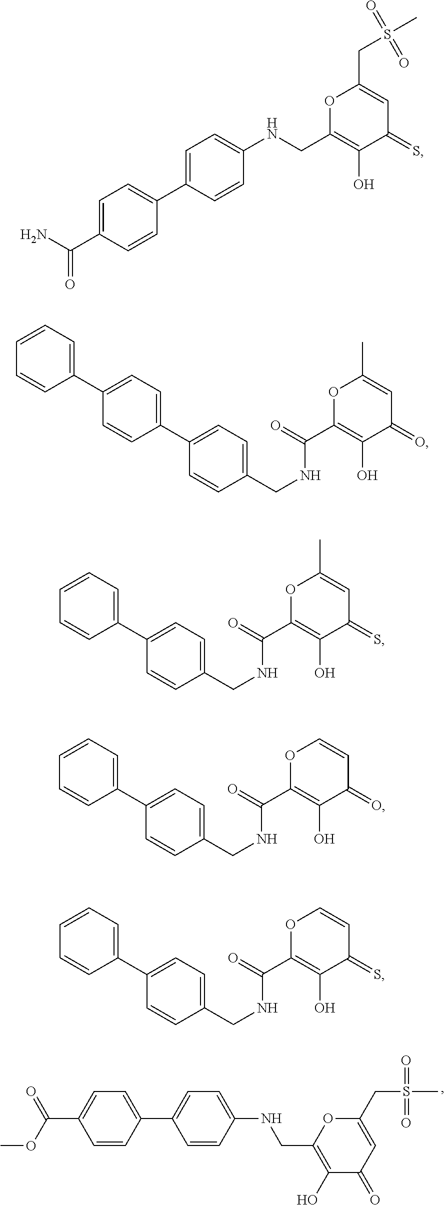

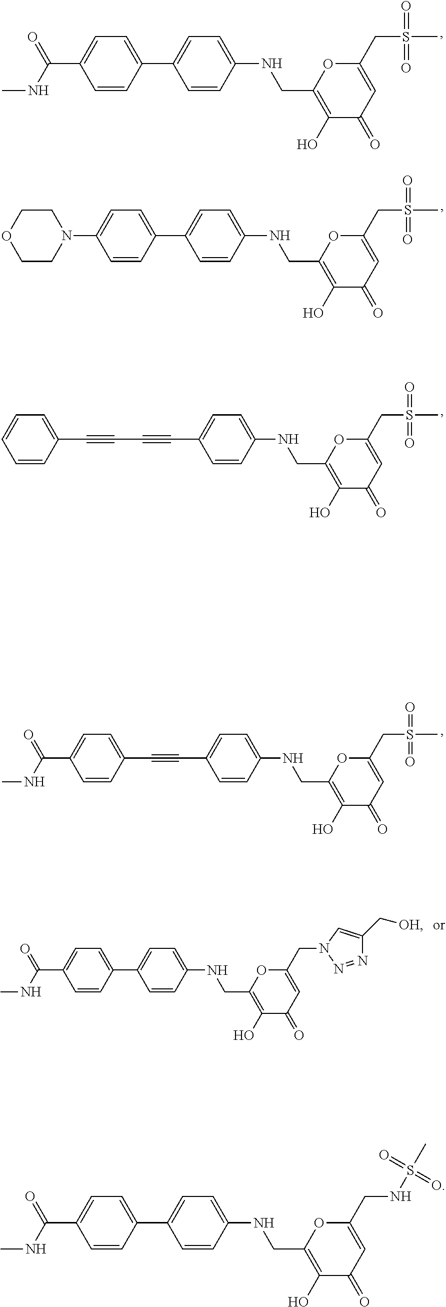

In embodiments, the compound is one or more of the following:

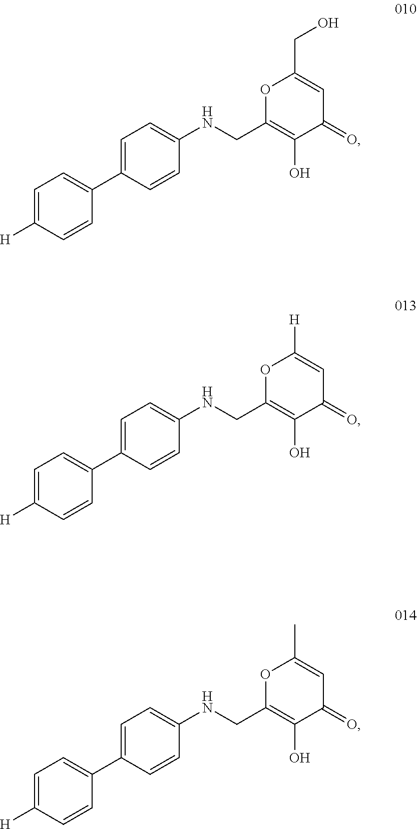 [SMILES: O=c1cc(CO)oc(CNc2ccc(-c3ccccc3)cc2)c1O ; O=c1ccoc(CNc2ccc(-c3ccccc3)cc2)c1O ; Cc1cc(=O)c(O)c(CNc2ccc(-c3ccccc3)cc2)o1]

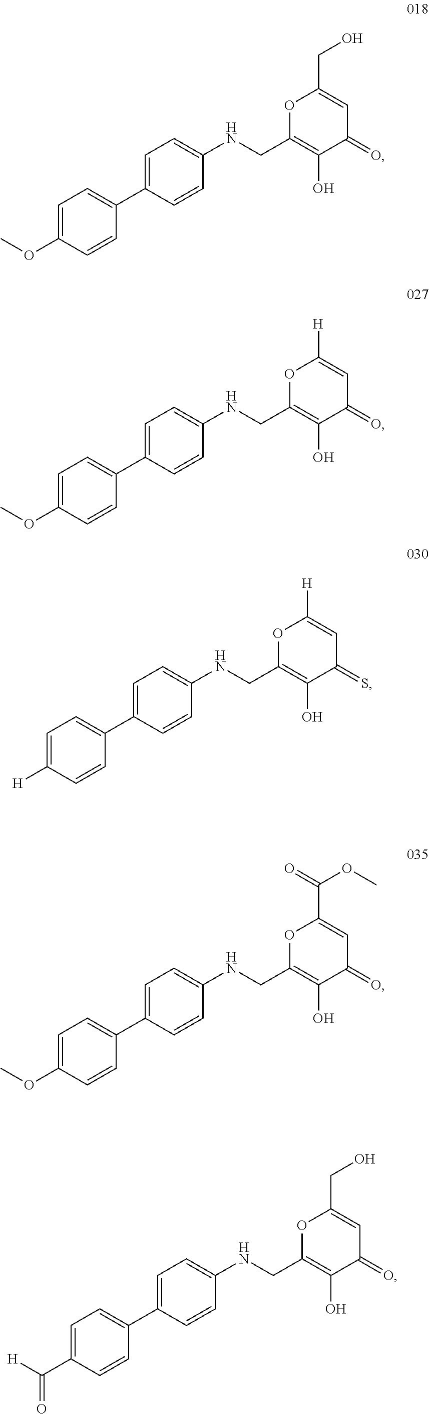

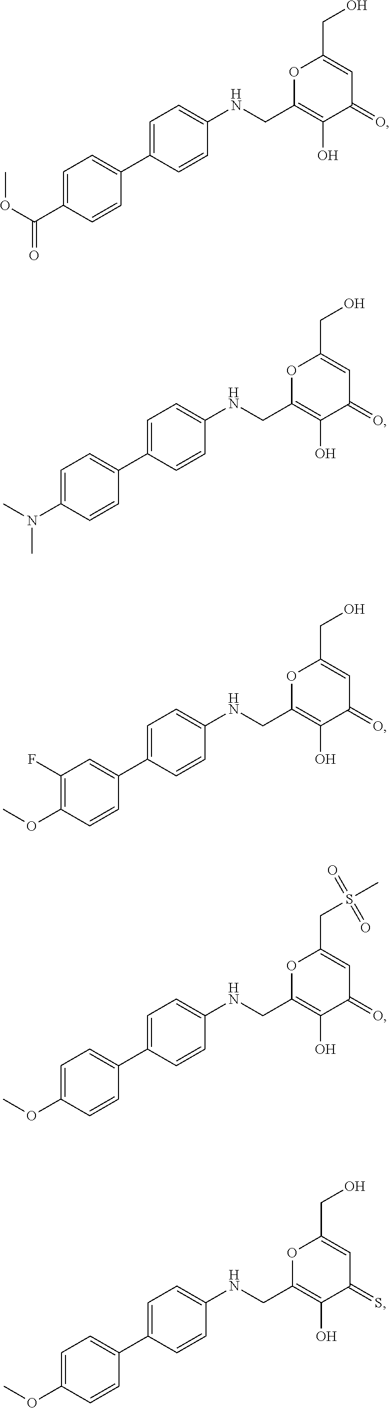

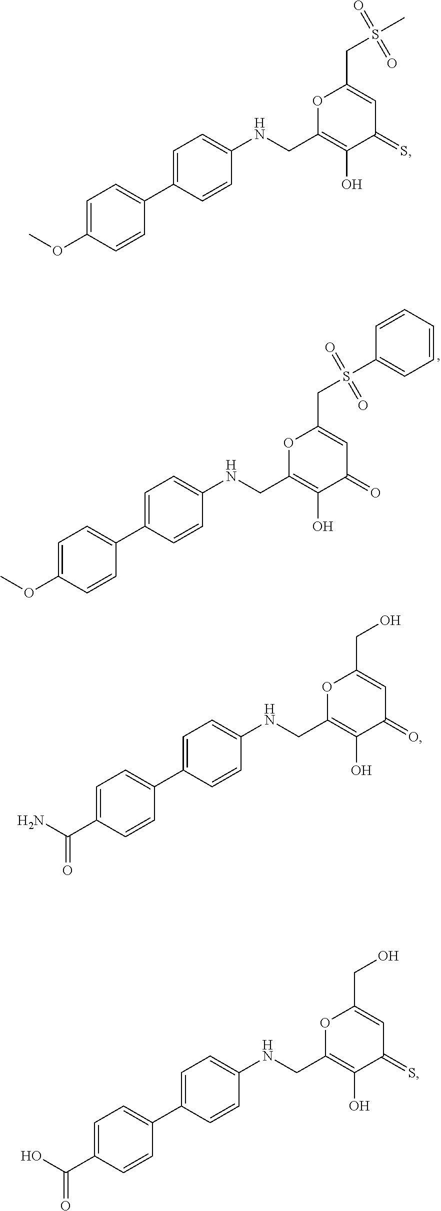

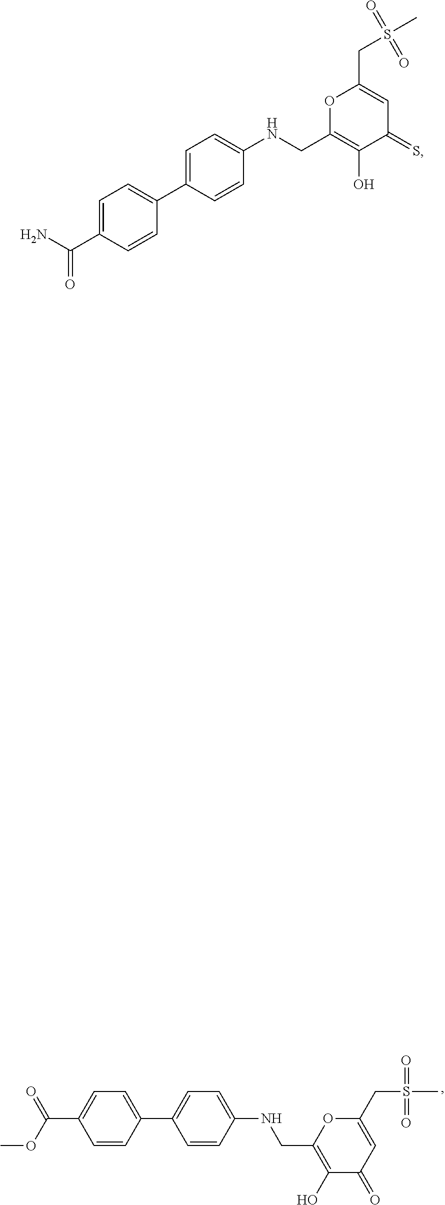

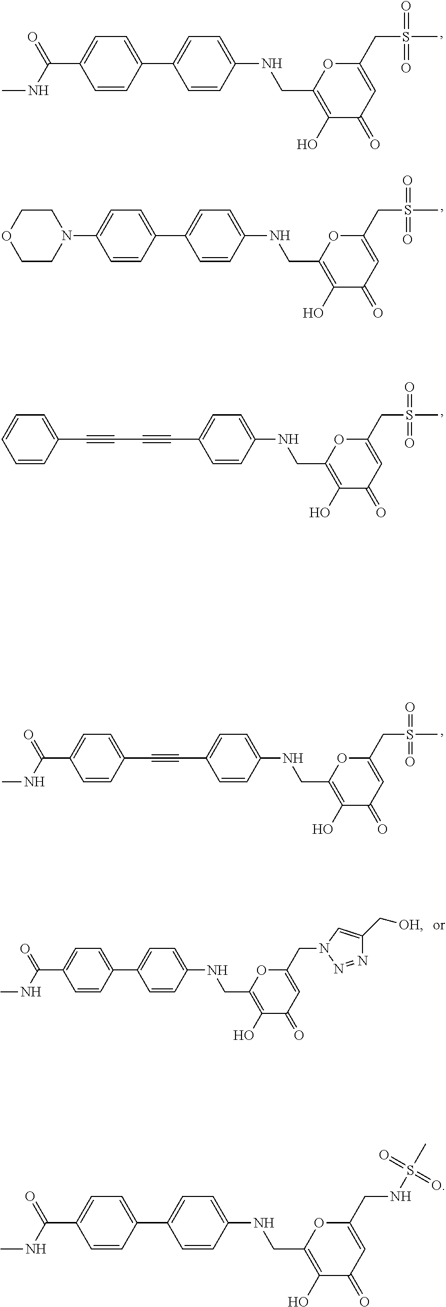

In embodiments, the compound has the structure of:

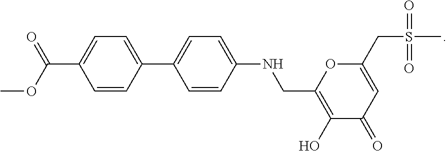

In embodiments, the compound has the structure of:

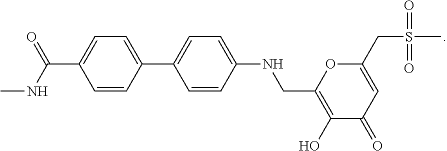

In embodiments, the compound has the structure of:

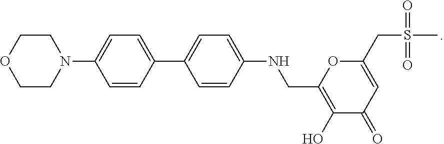 [SMILES: CS(=O)(=O)Cc1cc(=O)c(O)c(CNc2ccc(-c3ccc(N4CCOCC4)cc3)cc2)o1]

In embodiments, the compound has the structure of:

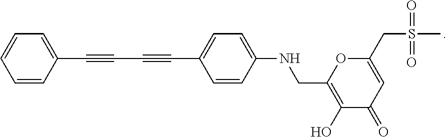 [SMILES: CS(=O)(=O)Cc1cc(=O)c(O)c(CNc2ccc(C#CC#Cc3ccccc3)cc2)o1]

In embodiments, the compound has the structure of:

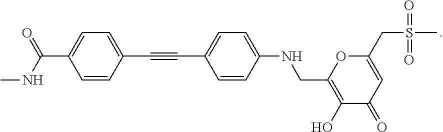

In embodiments, the compound has the structure of:

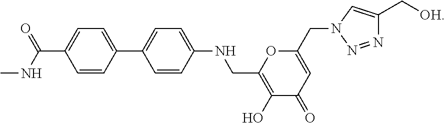 [SMILES: CNC(=O)c1ccc(-c2ccc(NCc3oc(Cn4cc(CO)nn4)cc(=O)c3O)cc2)cc1]

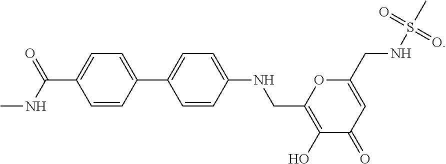 [SMILES: CNC(=O)c1ccc(-c2ccc(NCc3oc(CNS(C)(=O)=O)cc(=O)c3O)cc2)cc1]

### III. Pharmaceutical Compositions

In another aspect, there is provided a pharmaceutical composition including a pharmaceutically acceptable excipient and a compound described herein, e.g., compounds of formulae (I), (IA), (IB), (IC), (ID), (II), (III), (IV), (PI), (PII), and (PIII) as set forth herein, including embodiments thereof. In embodiments, the pharmaceutical composition include the compound of formula (IA) including embodiments thereof.

In embodiments, there is provided a pharmaceutical composition including a pharmaceutically acceptable excipient and a compound with structure of any one of formulae (PI), (PII) and (PIII) as set forth herein and embodiments thereof.

A. Formulations

The compounds of the present invention can be prepared and administered in a wide variety of oral, parenteral, and topical dosage forms. Thus, the compounds of the present invention can be administered by injection (e.g. intravenously, intramuscularly, intracutaneously, subcutaneously, intraduodenally, or intraperitoneally). Also, the compounds described herein can be administered by inhalation, for example, intranasally. Additionally, the compounds of the present invention can be administered transdermally. It is also envisioned that multiple routes of administration (e.g., intramuscular, oral, transdermal) can be used to administer the compounds of the invention. Accordingly, the present invention also provides pharmaceutical compositions comprising a pharmaceutically acceptable carrier or excipient and one or more compounds of the invention, i.e., “pharmaceutical formulation.”

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substance that may also act as diluents, flavoring agents, binders, preservatives, tablet disintegrating agents, or an encapsulating material.

In powders, the carrier is a finely divided solid in a mixture with the finely divided active component. In tablets, the active component is mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. The compounds of the invention can also be incorporated into liposomes or administered via transdermal pumps or patches. Pharmaceutical admixtures suitable for use in the present invention include those described, for example, in PHARMACEUTICAL SCIENCES (17th Ed., Mack Pub. Co., Easton, Pa.) and WO 96/05309, the teachings of both of which are hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.01 mg to 10000 mg, more typically 1.0 mg to 1000 mg, most typically 10 mg to 500 mg, according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The compositions of the present invention may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

B. Effective Dosages

In another aspect, there is provided a method for treating an infection. The method includes administering to a subject in need a therapeutically effective amount of a compound disclosed herein, including embodiments thereof.

Pharmaceutical compositions provided by the present invention include compositions wherein the active ingredient is contained in a therapeutically effective amount. The actual amount effective for a particular application will depend, inter alia, on the condition being treated. For example, when administered in methods to treat infection, such compositions will contain an amount of active ingredient effective to achieve the desired result (e.g., to treat an infection).

The dosage and frequency (single or multiple doses) of compound administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds of the invention.

For any compound described herein, the therapeutically effective amount can be initially determined from cell culture assays. Target concentrations will be those concentrations of active compound(s) that are capable of eliciting innate immune response as measured, for example, using the methods described.

Therapeutically effective amounts for use in humans may be determined from animal models. For example, a dose for humans can be formulated to achieve a concentration that has been found to be effective in animals. The dosage in humans can be adjusted by monitoring effectiveness and adjusting the dosage upwards or downwards, as described above.

Dosages may be varied depending upon the requirements of the patient and the compound being employed. The dose administered to a patient, in the context of the present invention, should be sufficient to affect a beneficial therapeutic response in the patient over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached. In one embodiment of the invention, the dosage range is 0.001% to 10% w/v. In another embodiment, the dosage range is 0.1% to 5% w/v.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

C. Toxicity

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch. 1, p.1, 1975. The exact formulation, route of administration, and dosage can be chosen by the individual physician in view of the patient's condition and the particular method in which the compound is used.

### IV. Method of Use

In another aspect, there is provided a method of treating a UDP-3-O—(R-3-hydroxymyristoyl)-N-acetylglucosamine deacetylase (LpxC)-mediated disease in a patient in need of such treatment. The method includes administering a therapeutically effective amount of a compound described herein, e.g., compounds of formulae (I), (IA), (IB), (IC), (ID), (II), (III), (IV), (PI), (PII), and (PIII) as set forth herein, including embodiments thereof. In embodiments, the compound of formula (IA) is administered). The term “LpxC-mediated disease” and the like refer to diseases and disorders which are treated by inhibition of LpcC. In embodiments, the disease is an infectious disease. In embodiments, the disease is caused by gram-negative bacteria.

In another aspect, there is provided a method of treating a UDP-3-O—(R-3-hydroxymyristoyl)-N-acetylglucosamine deacetylase (LpxC)-mediated disease in a patient in need of such treatment. The method includes administering a therapeutically effective amount of a LpxC inhibitor compound, wherein said LpxC inhibitor compound is a substituted or unsubstituted pyridinone, substituted or unsubstituted pyridinethione, substituted or unsubstituted hydroxypyridinone, substituted or unsubstituted hydroxypyridinethione, substituted or unsubstituted hydroxypyranone, substituted or unsubstituted hydroxypyranthione, substituted or unsubstituted pyrocatechol or substituted or unsubstituted pyridinol. The term “LpxC inhibitor compound” and the like refer to compounds which can disrupt the biological activity of LpxC, including e.g., compounds of formulae (I), (IA), (IB), (IC), (ID), (II), (III), (IV), (PI), (PII), and (PIII) as set forth herein, including embodiments thereof.

Further to any embodiment of method, in some embodiments the disease is an infectious disease. In embodiments, the disease is a bacterial disease. In embodiments, the disease is caused by gram-negative bacteria.

### V. Examples

### Example 1. Synthesis of 3-hydroxy-6-(hydroxymethyl)-2-(((4′-methoxy-[1,1′-biphenyl]-4-yl)amino)methyl)-4H-pyran-4-one

A representative synthetic scheme for synthesis of Compound Ex-1 is provided in the scheme following.

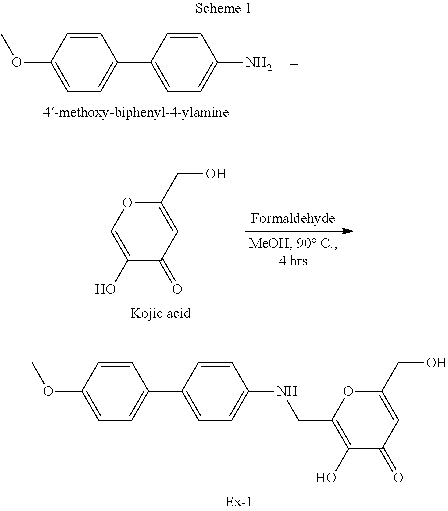 [SMILES: COc1ccc(-c2ccc(N)cc2)cc1 ; O=c1cc(CO)occ1O ; COc1ccc(-c2ccc(NCc3oc(CO)cc(=O)c3O)cc2)cc1]

To a solution of 4′-methoxy-biphenyl-4-ylamine (3.0 g, 15.06 mmol, 1.0 equiv) in Methanol (100 mL) was added 1.36 mL of a 37% (w/w) aqueous formaldehyde solution (18.07 mmol, 1.2 equiv.). The resulting solution was stirred at 70° C. for 30 min under N₂ in a 250 mL round bottom flask fitted with a reflux condenser. Kojic acid (2.57 g, 18.07 mmol, 1.2 equiv) was added to the cloudy light yellow solution as a solid and the reaction was stirred at reflux (85° C.) for 4 hours under N₂. The reaction mixture was concentrated in vacuo to a light orange-yellow residue and purified via flash silica column chromatography (Teledyne ISCO Combiflash Rf) eluting in 75% EtOAc in Hexanes to afford Cmpd Ex-1 an off-white solid, 3.0 g (56.4% yield). ¹HNMR (400 MHz, d-DMSO): δ=9.22 (br s, 1H), 7.44 (d, J=8.8 Hz), 7.32 (d, J=8.8 Hz), 6.91 (d, J=8.8 Hz), 6.69 (d, J=8.4 Hz), 6.28 (s, 1H, pyrone-H), 4.24 (m, 4H), 3.74 (s, 3H). ESI-MS(+): m/z 354.09 [M+H]+.

### Example 2. LpxC Inhibition in E. coli

Inhibition of LpxC from E. coli was assessed for compounds disclosed herein, shown in the table following. Methods for assessing inhibition of LpxC are known in the art.

TABLE 1
Inhibition of LpxC from E. coli

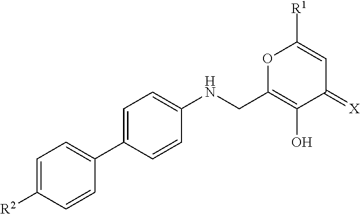

| | | | | % Inhibition |
|---|---|---|---|---|
| | | | | E. Coli LpxC |
| Compound | R¹ | R² | X | at 50 μM |
| 010 | CH₂OH | H | O | 39 |
| 013 | H | H | O | 67 |
| 014 | CH₃ | H | O | 23 |
| 018 | CH₂OH | OCH₃ | O | 81 |
| 027 | H | OCH₃ | O | 13 |
| 030 | CH₃ | H | S | 29 |
| 035 | COOCH₃ | OCH₃ | O | 29 |

TABLE 2
Inhibition of LpxC from E. coli was assessed for compounds
disclosed herein, shown in the table following.
Methods for assessing inhibition of LpxC are known in the art
(for reference: Brown et al. J. Med. Chem. 2012, 55, 914-923).

| | 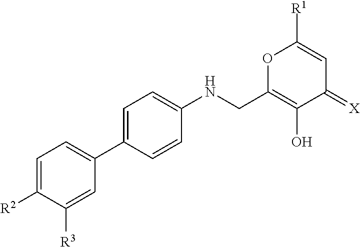 | | | | |
|---|---|---|---|---|---|
| | | | | | IC50 (μM) |
| | | | | | OR |
| | | | | | (% Inhibition |
| Com- | | | | | of E. Coli |
| pound | R¹ | R² | R³ | X | LpxC at 50 μM) |
| 010 | CH₂OH | H | H | O | (39) |
| 013 | H | H | H | O | (67) |
| 014 | CH₃ | H | H | O | (23) |
| 018 | CH₂OH | OCH₃ | H | O | 12.7 |
| 027 | H | OCH₃ | H | O | (13) |
| 030 | CH₃ | H | H | S | (29) |
| 035 | COOCH₃ | OCH₃ | H | O | (29) |
| 052 | CH₂OH | COOCH₃ | H | O | 1.4 |
| 053 | CH₂OH | N(CH₃)₂ | H | O | 2.5 |
| 054 | CH₂OH | OCH₃ | F | O | 6.1 |
| 058 | SO₂CH₃ | OCH₃ | H | O | 4.4 |
| 059 | CH₂OH | COOH | H | O | 67 |
| 085 | SO₂CH₃ | OCH₃ | H | S | 9.0 |
| 087 | SO₂CH₃ | COOCH₃ | H | O | 0.13 |
| 088 | SO₂CH₃ | morpholino | H | O | <0.10 |
| 090 | SO₂CH₃ | CONHCH₃ | H | O | <0.10 |

### Example 3. Further Synthetic Studies

Synthetic scheme for certain compounds disclosed herein is set forth in Scheme 2 following.

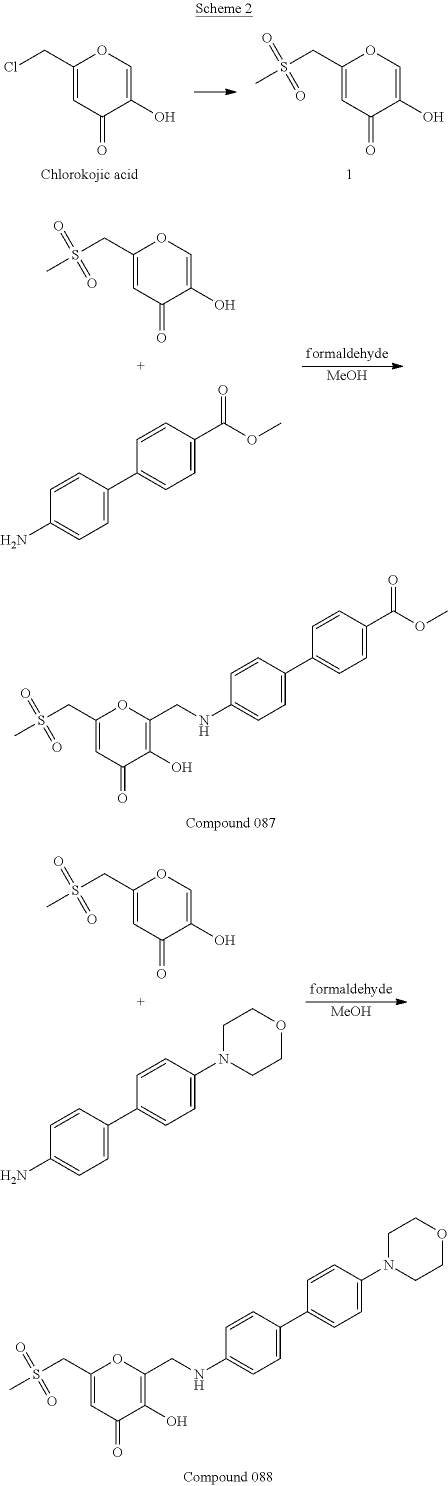

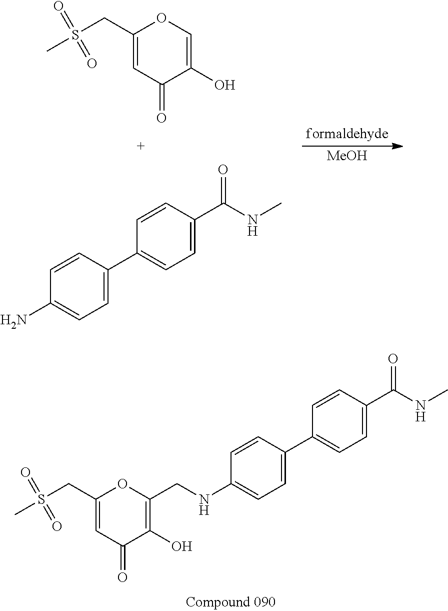

Compound 1: Chlorokojic acid was prepared as previously described (Liu et al. Bioorg. Med. Chem. 9 (2001) 563-573). A suspension of Chlorokojic acid (3 g, 18.7 mmol) and Sodium Methanesulfinate (2.5 g, 24.3 mmol) in water (15 mL) was irradiated in a microwave reactor at 120° C., 100 W, 50 psi for 30 minutes. Upon cooling to room temperature, the desired product crystallized out of solution as a light tan solid. Yield 72%. ¹HNMR (400 MHz, d-DMSO): δ=9.36 (br s, 1H), 8.10 (s, 1H), 6.49 (s, 1H), 4.60 (s, 2H), 3.1 (s, 3H). ESI-MS(−): m/z 203.20 [M−H]⁻.

Compound 087. To a solution of Methyl-4′-amino-[1,1′-biphenyl]-4-carboxylate (304 mg, 1.3 mmol, 1.0 equiv) in Methanol (35 mL) was added 0.131 mL of a 37% (w/w) aqueous formaldehyde solution (1.7 mmol, 1.3 equiv.). The resulting solution was stirred at 70° C. for 30 min under N₂ in a 100 mL round bottom flask fitted with a reflux condenser. Compound 1 (328 mg, 1.6 mmol, 1.2 equiv) was added to the solution as a solid and the reaction was stirred at reflux (80° C.) for 3 hours under N₂. The reaction mixture was concentrated in vacuo to a tan solid. The compound was purified via flash C18 column chromatography (Teledyne ISCO Combiflash Rf) eluting at 60% ACN/H2O in 0.1% formic acid to afford an off-white solid, 430 mg (73% yield). ¹HNMR (400 MHz, d-DMSO): δ=9.51 (br s, 1H), 7.92 (d, J=8.0 Hz, 2H), 7.69 (d, J=8.0 Hz, 2H), 7.50 (d, J=8.0 Hz, 2H), 6.73 (d, J=8.0 Hz, 2H), 6.55 (t, J=6.0 Hz, 1H), 6.47 (s, 1H), 4.55 (s, 2H), 4.30 (d, J=5.6 Hz, 2H), 3.83 (s, 3H), 3.00 (s, 3H). ESI-MS(−): m/z 442.11 [M−H]⁻.

Compound 088. To a solution of 4′-morpholino-[1,1′-biphenyl]-4-amine (245 mg, 0.96 mmol, 1.0 equiv) in Methanol (35 mL) was added 0.094 mL of a 37% (w/w) aqueous formaldehyde solution (1.3 mmol, 1.3 equiv.). The resulting solution was stirred at 70° C. for 30 min under N₂ in a 100 mL round bottom flask fitted with a reflux condenser. Compound 1 (236 mg, 1.16 mmol, 1.2 equiv) was added to the solution as a solid and the reaction was stirred at reflux (80° C.) for 2 hours under N₂. The reaction mixture was concentrated in vacuo to a tan solid. The compound was purified via flash C18 column chromatography (Teledyne ISCO Combiflash Rf) eluting at 50% ACN/H2O in 0.1% formic acid to afford an off-white solid, 230 mg (51% yield). ¹HNMR (400 MHz, d-DMSO): δ=9.46 (br s, 1H), 7.39 (d, J=8.8 Hz, 2H), 7.31 (d, J=8.8 Hz, 2H), 6.92 (d, J=8.8 Hz, 2H), 6.66 (d, J=8.8 Hz, 2H), 6.46 (s, 1H), 6.22 (t, J=6.0 Hz, 1H) 4.55 (s, 2H), 4.26 (d, J=6.0 Hz, 2H), 3.72 (t, J=4.6 Hz, 4H), 3.07 (t, J=4.6 Hz, 4H), 3.00 (s, 3H). ESI-MS(+): m/z 471.23 [M+H]⁺.

Compound 090. To a solution of 4′-amino-N-methyl-[1,1′-biphenyl]-4-carboxamide (200 mg, 0.88 mmol, 1.0 equiv) in Methanol (20 mL) was added 0.086 mL of a 37% (w/w) aqueous formaldehyde solution (1.2 mmol, 1.3 equiv.). The resulting solution was stirred at 70° C. for 30 min under N₂ in a 100 mL round bottom flask fitted with a reflux condenser. Compound 1 (217 mg, 1.06 mmol, 1.2 equiv) was added to the clear light yellow solution as a solid and the reaction was stirred at reflux (80° C.) for 5 hours under N₂. The reaction mixture was concentrated in vacuo to a light brown oil, dissolved in 50 mL 0.5M NaOH and washed with 3×20 mL EtOAc. The aqueous phase was neutralized to pH 7 with 1M HCl and concentrated in vacuo. The compound was purified via flash C18 column chromatography (Teledyne ISCO Combiflash Rf) eluting at 65% MeOH/H2O in 0.1% formic acid to afford an off-white solid, 150 mg (38% yield). ¹HNMR (400 MHz, d-DMSO): δ=8.34 (br s, 1H), 7.81 (d, J=8.4 Hz, 2H), 7.61 (d, J=8.4 Hz, 2H), 7.47 (d, J=8.4 Hz, 2H), 6.72 (d, J=8.4 Hz, 2H), 6.47 (s, 1H), 4.76 (br s, 1H), 4.56 (s, 2H), 4.30 (s, 2H), 3.00 (s, 3H), 2.76 (d, J=4.4 Hz, 2H). ESI-MS(+): m/z 443.08 [M+H]⁺.

### Embodiments

Embodiments include embodiments 1-214 following:

### Embodiment 1

A compound of formula:

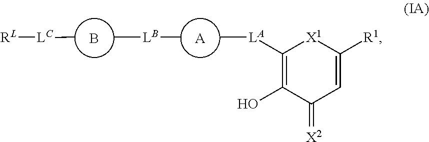

wherein R¹ is independently hydrogen, halogen, —CX^{a}₃, —CN, —SR³, —SO₂Cl, —SOₙ₁R³, —SOᵥ₁NR³R⁴, —NHNH₂, —ONR³R⁴, —NHC═(O)NHNH₂, —NHC═(O)NR³R⁴, —N(O)ₘ₁, —NR³R⁴, —NH—O—R³, —C(O)R³, —C(O)—OR³, —C(O)NR³R⁴, —OR³, -L⁴-SOₙ₁R³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{L} is independently hydrogen, halogen, —CX^{b}₃, —CN, —SR⁵, —SO₂Cl, —SOₙR⁵, —SOₙ₂NR⁵R⁶, —NHNH₂, —ONR⁵R⁶, —NHC═(O)NHNH₂, —NHC═(O)NR⁵R⁶, —N(O)ₘ₂, —NR⁵R⁶, —NH—O—R⁵, —C(O)R⁵, —C(O)—OR⁵, —C(O)NR⁵R⁶, —OR⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R³, R⁴, R⁵, R⁶ and R⁷ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L^{A} is independently a bond, —S(O)—, —S(O)₂NH—, —NHS(O)₂—, —C(O)O—, —OC(O)—, —C(O)—, —C(O)NH—, —C(O)NH-L⁴-, -L⁴-C(O)NH—, —NH—, —NHC(O)—, —NH-L⁴-, -L⁴-NH—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L^{B} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L^{C} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L⁴ is independently —C(O)—, —C(O)—NH—, —NH—C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; ring A is substituted or unsubstituted cycloalkylene, substituted or unsubstituted heterocycloalkylene, substituted or unsubstituted arylene, or substituted or unsubstituted heteroarylene; ring B is substituted or unsubstituted cycloalkylene, substituted or unsubstituted heterocycloalkylene, substituted or unsubstituted arylene, or substituted or unsubstituted heteroarylene; X¹ is C, N, O, NR⁷ or S; X² is independently O or S; X^{a} and X^{b} are independently F, Cl, Br, or I; n₁ and n₂ are independently an integer from 0 to 4; m₁ and m₂ are independently an integer from 1 to 2; and v₁ and v₂ are independently an integer from 1 to 2; wherein if L^{A} is —C(O)NH-L⁴, L⁴ is methylene, ring A and ring B are each unsubstituted phenyl, L^{C} is a bond or —O—, and X¹ is O, then R^{L} is not hydrogen.

### Embodiment 2

The compound of embodiment 1, wherein R¹ is independently R^{1A-}substituted or unsubstituted alkyl, R^{1A}-substituted or unsubstituted heteroalkyl, R^{1A}-substituted or unsubstituted cycloalkyl, R^{1A}-substituted or unsubstituted heterocycloalkyl, R^{1A}-substituted or unsubstituted aryl, or R^{1A}-substituted or unsubstituted heteroaryl; R^{1A} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1B}-substituted or unsubstituted alkyl, R^{1B}-substituted or unsubstituted heteroalkyl, R^{1B}-substituted or unsubstituted cycloalkyl, R^{1B}-substituted or unsubstituted heterocycloalkyl, R^{1B}-substituted or unsubstituted aryl, or R^{1B}-substituted or unsubstituted heteroaryl; R^{1B} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1C}-substituted or unsubstituted alkyl, R^{1C}-substituted or unsubstituted heteroalkyl, R^{1C}-substituted or unsubstituted cycloalkyl, R^{1C}-substituted or unsubstituted heterocycloalkyl, R^{1C}-substituted or unsubstituted aryl, or R^{1C}-substituted or unsubstituted heteroaryl; R^{1C} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl.

### Embodiment 3

The compound of embodiment 1, wherein R^{L} is independently R^{LA}-substituted or unsubstituted alkyl, R^{LA}-substituted or unsubstituted heteroalkyl, R^{LA}-substituted or unsubstituted cycloalkyl, R^{LA}-substituted or unsubstituted heterocycloalkyl, R^{LA}-substituted or unsubstituted aryl, or R^{LA}-substituted or unsubstituted heteroaryl; R^{LA} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{LB}-substituted or unsubstituted alkyl, R^{LB}-substituted or unsubstituted heteroalkyl, R^{LB}-substituted or unsubstituted cycloalkyl, R^{LB}-substituted or unsubstituted heterocycloalkyl, R^{LB}-substituted or unsubstituted aryl, or R^{LB}-substituted or unsubstituted heteroaryl; R^{LB} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{LC}-substituted or unsubstituted alkyl, R^{LC}-substituted or unsubstituted heteroalkyl, R^{LC}-substituted or unsubstituted cycloalkyl, R^{LC}-substituted or unsubstituted heterocycloalkyl, R^{LC}-substituted or unsubstituted aryl, or R^{LC}-substituted or unsubstituted heteroaryl; R^{LC} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl.

### Embodiment 4

The compound of any one of embodiments 1-3, wherein R¹ is independently hydrogen, —C(O)—OR³, -L⁴-SOₙ₁R³ or substituted or unsubstituted alkyl.

### Embodiment 5

The compound of one of embodiments 1 to 4, wherein R¹ is hydrogen.

### Embodiment 6

The compound of one of embodiments 1 to 4, wherein R¹ is —C(O)—OR³ and R³ is substituted or unsubstituted C₁-C₁₀ alkyl.

### Embodiment 7

The compound of one of embodiments 1 to 4 or 6, wherein R³ is substituted or unsubstituted C₁-C₅ alkyl.

### Embodiment 8

The compound of one of embodiments 1 to 4 or 6, wherein R³ is unsubstituted C₁-C₅ alkyl.

### Embodiment 9

The compound of one of embodiments 1 to 4 or embodiment 6, wherein R³ is methyl.

### Embodiment 10

The compound of one of embodiments 1 to 4, wherein R¹ is -L⁴-SOₙ₁R³, R³ is substituted or unsubstituted alkyl or substituted or unsubstituted aryl, and L⁴ is substituted or unsubstituted alkylene.

### Embodiment 11

The compound of one of embodiments 1 to 4 or 10, wherein R³ is substituted or unsubstituted C₁-C₁₀ alkyl or substituted or unsubstituted C₅-C₆ aryl.

### Embodiment 12

The compound of one of embodiments 1 to 4, 10 or 11, wherein R³ is substituted or unsubstituted C₁-C₅ alkyl.

### Embodiment 13

The compound of one of embodiments 1 to 4 or 10-12, wherein R³ is unsubstituted C₁-C₅ alkyl.

### Embodiment 14

The compound of one of embodiments 1 to 4 or 10-13, wherein R³ is methyl.

### Embodiment 15

The compound of one of embodiments 1 to 4, 10 or 11, wherein R³ is unsubstituted C₅-C₆ aryl.

### Embodiment 16

The compound of one of embodiments 1 to 4, 10, 11 or 15, wherein R³ is unsubstituted phenyl.

### Embodiment 17

The compound of any one of embodiments 10-16, wherein L⁴ is substituted or unsubstituted C₁-C₁₀ alkylene.

### Embodiment 18

The compound of any one of embodiments 10-16, wherein L⁴ is substituted or unsubstituted C₁-C₅ alkylene.

### Embodiment 19

The compound of any one of embodiments 10-16, wherein L⁴ is unsubstituted C₁-C₅ alkylene.

### Embodiment 20

The compound of any one of embodiments 10-16, wherein L⁴ is methylene.

### Embodiment 21

The compound of one of embodiments 1 to 4, wherein R¹ is substituted or unsubstituted C₁-C₁₀ alkyl.

### Embodiment 22

The compound of embodiment 21, wherein R¹ is substituted or unsubstituted C₁-C₅ alkyl.

### Embodiment 23

The compound of embodiment 22, wherein R¹ unsubstituted C₁-C₅ alkyl.

### Embodiment 24

The compound of embodiment 23, wherein R¹ is methyl.

### Embodiment 25

The compound of embodiment 22 wherein R¹ is R^{1A}-substituted C₁-C₅ alkyl.

### Embodiment 26

The compound of embodiment 25, wherein R¹ is R^{1A}-substituted C₁ alkyl and R^{1A} is —OH.

### Embodiment 27

The compound of any one of embodiments 1-26, wherein R^{L} is independently hydrogen, halogen, —OR⁵, —C(O)—OR⁵, —NR⁵R⁶, —C(O)NR⁵R⁶, substituted or unsubstituted heterocycloalkyl or substituted or unsubstituted aryl.

### Embodiment 28

The compound of any one of embodiments 1-26, wherein R^{L} is hydrogen.

### Embodiment 29

The compound of any one of embodiments 1-26, wherein R^{L} is halogen.

### Embodiment 30

The compound of any one of embodiments 1-26, wherein R^{L} is —F.

### Embodiment 31

The compound of any one of embodiments 1-26, wherein R^{L} is —OR⁵ and R⁵ is substituted or unsubstituted C₁-C₁₀ alkyl.

### Embodiment 32

The compound of any one of embodiments 1-27 or 31, wherein R⁵ is substituted or unsubstituted C₁-C₅ alkyl.

### Embodiment 33

The compound of any one of embodiments 1-27 or 31, wherein R⁵ is unsubstituted C₁-C₅ alkyl.

### Embodiment 34

The compound of any one of embodiments 1-27 or 31, wherein R⁵ is methyl.

### Embodiment 35

The compound of any one of embodiments 1-26, wherein R^{L} is —C(O)—OR⁵ and R⁵ is hydrogen or substituted or unsubstituted C₁-C₁₀ alkyl.

### Embodiment 36

The compound of any one of embodiments 1-27 or 35, wherein R⁵ is hydrogen.

### Embodiment 37

The compound of any one of embodiments 1-27 or 35, wherein R⁵ is substituted or unsubstituted C₁-C₅ alkyl.

### Embodiment 38

The compound of any one of embodiments 1-27 or 35, wherein R⁵ is unsubstituted C₁-C₅ alkyl.

### Embodiment 39

The compound of any one of embodiments 1-27 or 35, wherein R⁵ is methyl.

### Embodiment 40

The compound of any one of embodiments 1-27, wherein R^{L} is —NR⁵R⁶ and R⁵ and R⁶ are independently hydrogen or substituted or unsubstituted C₁-C₁₀ alkyl.

### Embodiment 41

The compound of any one of embodiments 1-27 or 40, wherein R⁵ and R⁶ are independently hydrogen or substituted or unsubstituted C₁-C₅ alkyl.

### Embodiment 42

The compound of any one of embodiments 1-27 or 40, wherein R⁵ and R⁶ are independently unsubstituted C₁-C₅ alkyl.

### Embodiment 43

The compound of any one of embodiments 1-27 or 40, wherein R⁵ and R⁶ are methyl.

### Embodiment 44

The compound of any one of embodiments 1-27 or 40, wherein R^{L} is —C(O)NR⁵R⁶ and R⁵ and R⁶ are independently hydrogen or substituted or unsubstituted C₁-C₁₀ alkyl.

### Embodiment 45

The compound of any one of embodiments 1-27 or 40, wherein R⁵ and R⁶ are independently hydrogen or substituted or unsubstituted C₁-C₅ alkyl.

### Embodiment 46

The compound of any one of embodiments 1-27 or 40, wherein R⁵ and R⁶ are independently hydrogen or unsubstituted C₁-C₅ alkyl.

### Embodiment 47

The compound of any one of embodiments 1-27 or 40, wherein R⁵ and R⁶ are independently hydrogen or methyl.

### Embodiment 48

The compound of any one of embodiments 1-27 or 40, wherein R⁵ and R⁶ are hydrogen.

### Embodiment 49

The compound of any one of embodiments 1-27, wherein R^{L} is substituted or unsubstituted 5-6 membered heterocycloalkyl.

### Embodiment 50

The compound of any one of embodiments 1-27 or 49, wherein R^{L} is unsubstituted 6 membered heterocycloalkyl.

### Embodiment 51

The compound of any one of embodiments 1-27 or 49, wherein R^{L} is unsubstituted morpholinyl.

### Embodiment 52

The compound of any one of embodiments 1-27, wherein R^{L} is substituted or unsubstituted C₅-C₆ aryl.

### Embodiment 53

The compound of any one of embodiments 1-27 or 52, wherein R^{L} is substituted or unsubstituted C₆ aryl.

### Embodiment 54

The compound of any one of embodiments 1-27 or 52, wherein R^{L} is unsubstituted phenyl.

### Embodiment 55

The compound of any one of embodiments 1-54, wherein L^{A} is —C(O)NH-L⁴- or -L⁴-NH—.

### Embodiment 56

The compound of any one of embodiments 1-54 or 55, wherein L^{A} is —C(O)NH-L⁴- and L⁴ is substituted or unsubstituted C₁-C₁₀ alkylene.

### Embodiment 57

The compound of any one of embodiments 1-54 or 56, wherein L⁴ is substituted or unsubstituted C₁-C₅ alkylene.

### Embodiment 58

The compound of any one of embodiments 1-54 or 56, wherein L⁴ is unsubstituted C₁-C₅ alkylene.

### Embodiment 59

The compound of any one of embodiments 1-54 or 56, wherein L⁴ is unsubstituted C₁-C₃ alkylene.

### Embodiment 60

The compound of any one of embodiments 1-54 or 56, wherein L⁴ is methylene.

### Embodiment 61

The compound of any one of embodiments 1-55, wherein L^{A} is -L⁴-NH— and L⁴ is substituted or unsubstituted C₁-C₁₀ alkylene.

### Embodiment 62

The compound of any one of embodiments 1-55 or 61, wherein L⁴ is substituted or unsubstituted C₁-C₅ alkylene.

### Embodiment 63

The compound of any one of embodiments 1-55 or 61, wherein L⁴ is unsubstituted C₁-C₅ alkylene.

### Embodiment 64

The compound of any one of embodiments 1-55 or 61, wherein L⁴ is unsubstituted C₁-C₃ alkylene.

### Embodiment 65

The compound of any one of embodiments 1-55 or 61, wherein L⁴ is methylene.

### Embodiment 66

The compound of any one of embodiments 1-65, wherein L^{B} is independently a bond or unsubstituted C₁-C₁₀ alkylene.

### Embodiment 67

The compound of any one of embodiments 1-65 or 66, wherein L^{B} is a bond.

### Embodiment 68

The compound of any one of embodiments 1-65 or 66, wherein L^{B} is unsubstituted C₁-C₅ alkylene.

### Embodiment 69

The compound of any one of embodiments 1-65 or 66, wherein L^{B} is unsaturated unsubstituted C₁-C₅ alkylene.

### Embodiment 70

The compound of any one of embodiments 1-65 or 66, wherein L^{B} is unsaturated unsubstituted C₁-C₃ alkylene.

### Embodiment 71

The compound of any one of embodiments 1-65 or 66, wherein L^{B} is —C≡C—.

### Embodiment 72

The compound of any one of embodiments 1-71, wherein L^{C} is a bond.

### Embodiment 73

The compound of any one of embodiments 1-72, wherein ring A is substituted or unsubstituted C₅-C₆ arylene.

### Embodiment 74

The compound of any one of embodiments 1-72 or 73, wherein ring A is substituted or unsubstituted C₆ arylene.

### Embodiment 75

The compound of any one of embodiments 1-72 or 73, wherein ring A is unsubstituted phenylene.

### Embodiment 76

The compound of any one of embodiments 1-75, wherein ring B is substituted or unsubstituted C₅-C₆ arylene.

### Embodiment 77

The compound of any one of embodiments 1-75 or 76, wherein ring B is substituted or unsubstituted C₆ arylene.

### Embodiment 78

The compound of any one of embodiments 1-75 or 76, wherein ring B is unsubstituted phenylene.

### Embodiment 79

The compound of embodiment 1, wherein said compound has the formula:

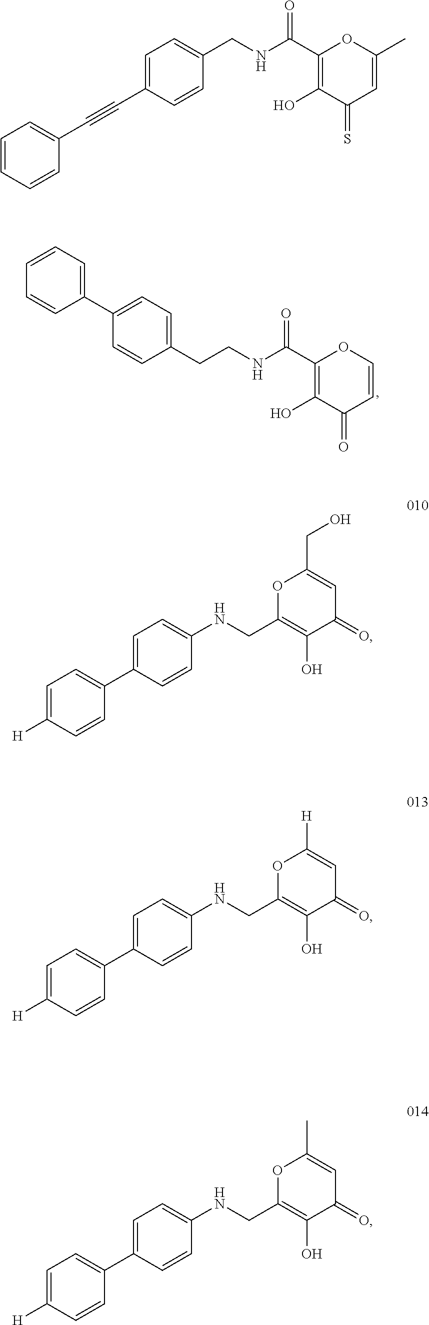 [SMILES: Cc1cc(=S)c(O)c(C(=O)NCc2ccc(C#Cc3ccccc3)cc2)o1 ; O=C(NCCc1ccc(-c2ccccc2)cc1)c1occc(=O)c1O ; O=c1cc(CO)oc(CNc2ccc(-c3ccccc3)cc2)c1O ; O=c1ccoc(CNc2ccc(-c3ccccc3)cc2)c1O ; Cc1cc(=O)c(O)c(CNc2ccc(-c3ccccc3)cc2)o1]

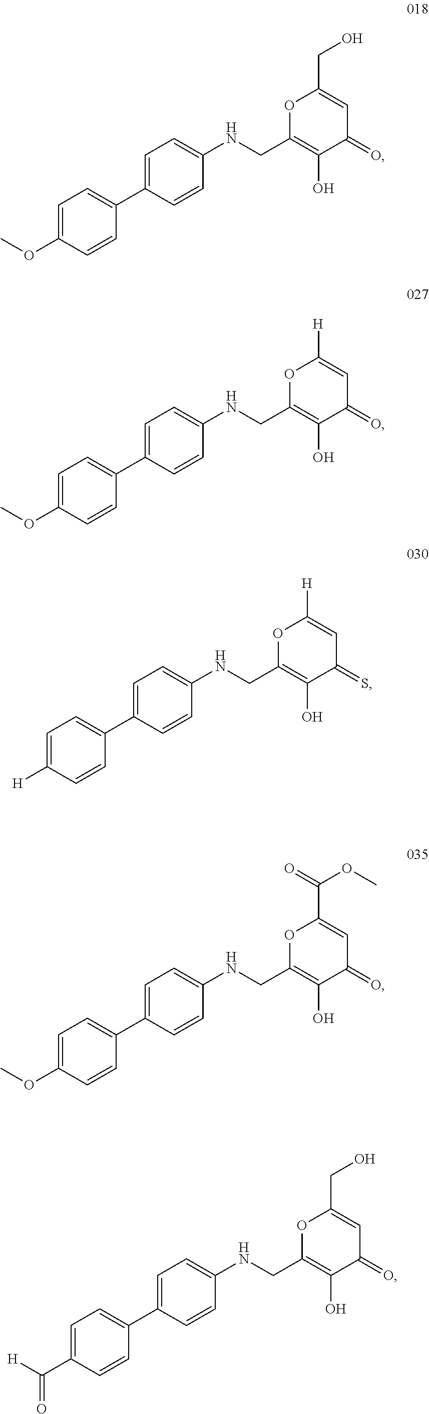

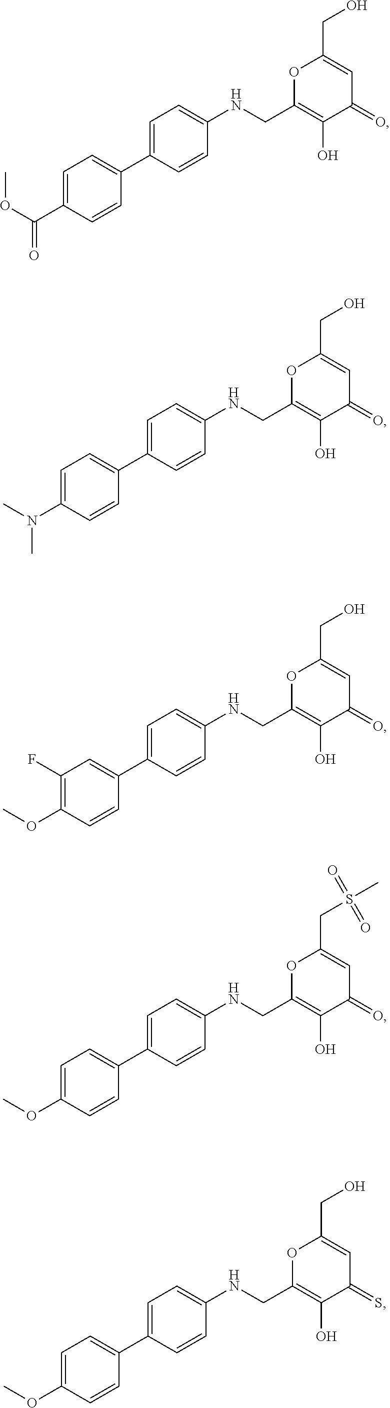

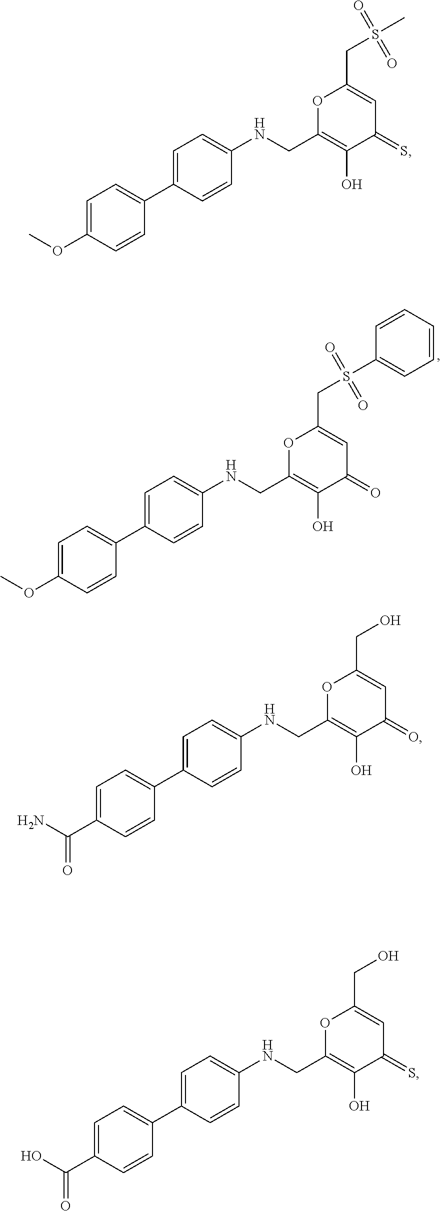

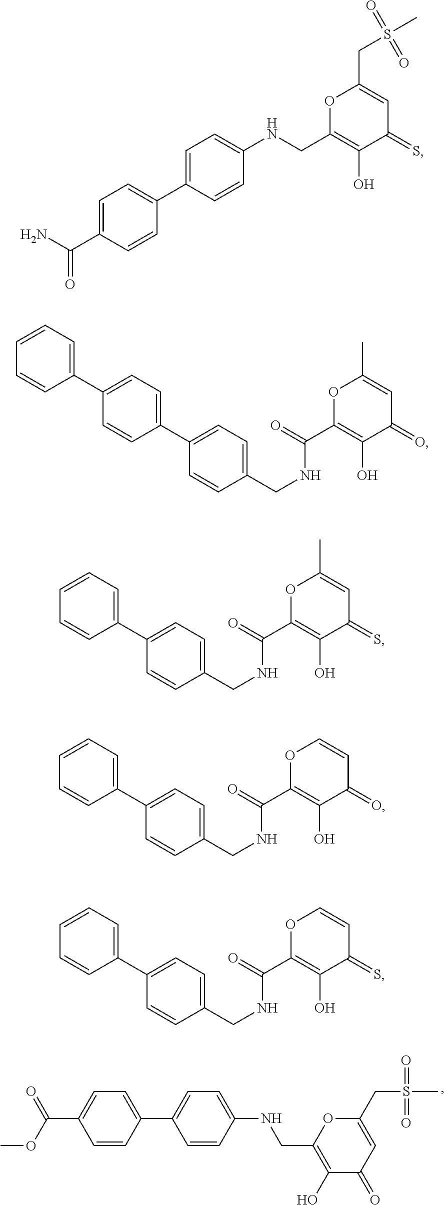

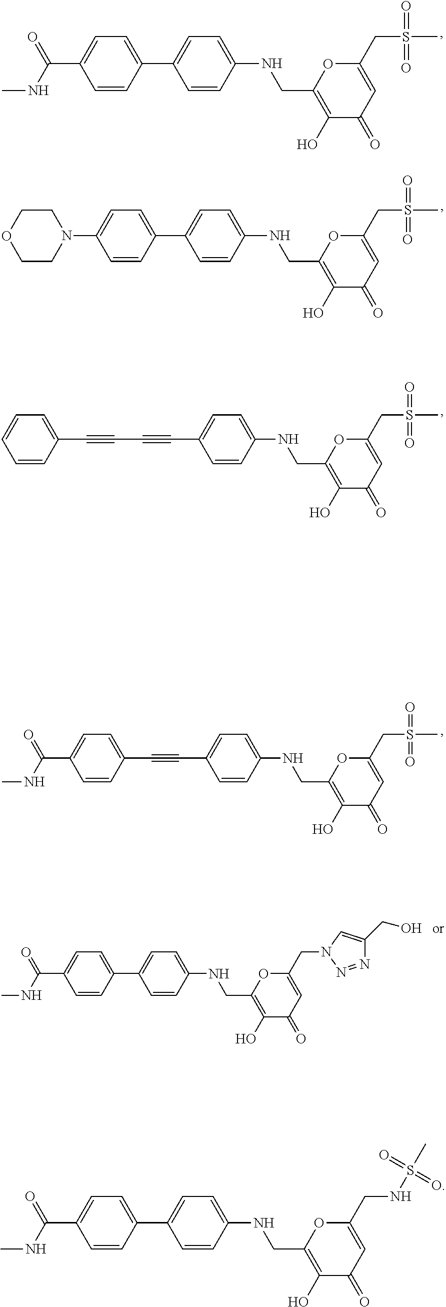

### Embodiment 80

A pharmaceutical composition comprising a pharmaceutically acceptable excipient and a compound of any one of embodiments 1-79.

### Embodiment 81

A method of treating a UDP-3-O—(R-3-hydroxymyristoyl)-N-acetylglucosamine deacetylase (LpxC)-mediated disease in a patient in need of such treatment, said method comprising administering a therapeutically effective amount of a compound of any one of embodiments 1-79.

### Embodiment 82

The method of embodiment 81, wherein said disease is an infectious disease.

### Embodiment 83

The method of embodiment 81, wherein said disease is caused by gram-negative bacteria.

### Embodiment 84

A method of treating a UDP-3-O—(R-3-hydroxymyristoyl)-N-acetylglucosamine deacetylase (LpxC)-mediated disease in a patient in need of such treatment, said method comprising administering a therapeutically effective amount of a LpxC inhibitor compound, wherein said LpxC inhibitor compound is a substituted or unsubstituted pyridinone, substituted or unsubstituted pyridinethione, substituted or unsubstituted hydroxypyridinone, substituted or unsubstituted hydroxypyridinethione, substituted or unsubstituted hydroxypyranone, substituted or unsubstituted hydroxypyranthione, substituted or unsubstituted pyrocatechol or substituted or unsubstituted pyridinol.

### Embodiment 85

The method of embodiment 84, wherein said compound has the formula:

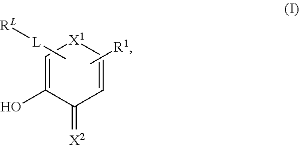

wherein R¹ is independently hydrogen, halogen, —CX^{a}₃, —CN, —SR³, —SO₂Cl, —SOₙ₁R³, —SOᵥ₁NR³R⁴, —NHNH₂, —ONR³R⁴, —NHC═(O)NHNH₂, —NHC═(O)NR³R⁴, —N(O)ₘ₁, —NR³R⁴, —NH—O—R³, —C(O)R³, —C(O)—OR³, —C(O)NR³R⁴, —OR³, -L⁴-SOₙ₁R³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{L} is independently hydrogen, halogen, —CX^{b}₃, —CN, —SR, —SO₂Cl, —SOₙ₂R, —SOᵥ₂NR⁵R⁶, —NHNH₂, —ONR⁵R⁶, —NHC═(O)NHNH₂, —NHC═(O)NR⁵R⁶, —N(O)ₘ₂, —NR⁵R⁶, —NH—O—R⁵, —C(O)R⁵, —C(O)—OR⁵, —C(O)NR⁵R⁶, —OR⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R³, R⁴, R⁵, R⁶ and R⁷ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L is independently a bond, —S(O)—, —S(O)₂NH—, —NHS(O)₂—, —C(O)O—, —OC(O)—, —C(O)—, —C(O)NH—, —C(O)NH-L⁵-, -L⁵-C(O)NH—, —NH—, —NHC(O)—, —NH-L⁵-, -L⁵-NH—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L⁵ is independently —C(O)—, —C(O)—NH₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; X^{a} and X^{b} are independently —F, —Cl, —Br, or —I; n₁ and n₂ are independently an integer from 0 to 4; m₁ and m₂ are independently an integer from 1 to 2; v₁ and v₂ are independently an integer from 1 to 2; X¹ is C, N, O, NR⁷ or S; and X² is O or S.

### Embodiment 86

The method of embodiment 85, wherein L is

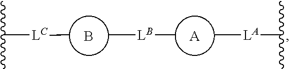

wherein L^{A} is independently a bond, —S(O)—, —S(O)₂NH—, —NHS(O)₂—, —C(O)O—, —OC(O)—, —C(O)—, —C(O)NH—, —C(O)NH-L⁴-, —NH—, —NHC(O)—, —NH-L⁴-, -L⁴-NH—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L^{B} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L⁴ is independently —C(O)—, —C(O)—NH₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; ring A is substituted or unsubstituted cycloalkylene, substituted or unsubstituted heterocycloalkylene, substituted or unsubstituted arylene, or substituted or unsubstituted heteroarylene; and ring B is substituted or unsubstituted cycloalkylene, substituted or unsubstituted heterocycloalkylene, substituted or unsubstituted arylene, or substituted or unsubstituted heteroarylene.

### Embodiment 87

The method of embodiment 85 or 86, wherein said compound has the formula:

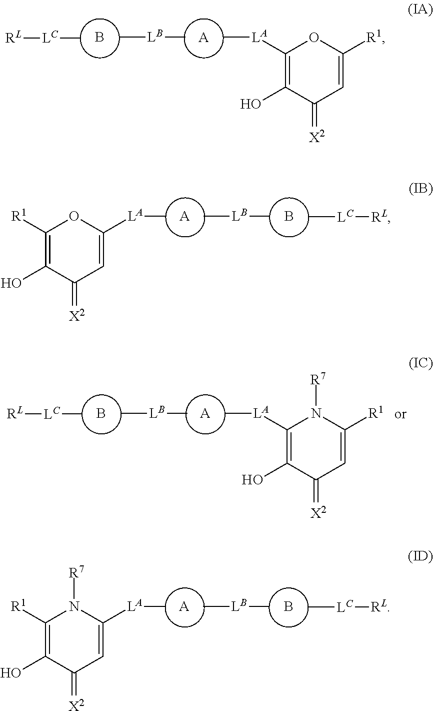

### Embodiment 88

The method of embodiment 87, wherein said compound has the formula:

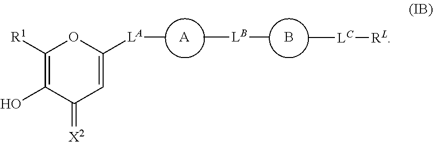

### Embodiment 89

The method of embodiment 88, wherein R¹ is hydrogen.

### Embodiment 90

The method of embodiment 88 or 89, wherein R^{L} is independently hydrogen, —CN or substituted or unsubstituted heterocycloalkyl.

### Embodiment 91

The method of embodiment 90, wherein R^{L} is substituted or unsubstituted 5-6 membered heterocycloalkyl.

### Embodiment 92

The method of embodiment 91, wherein R^{L} is unsubstituted 6 membered heterocycloalkyl.

### Embodiment 93

The method of embodiment 92, wherein R^{L} is unsubstituted morpholinyl.

### Embodiment 94

The method of any one of embodiments 88-93, wherein L^{A} is independently —C(O)NH-L⁴-, -L⁴-NH— or substituted or unsubstituted heteroalkylene.

### Embodiment 95

The method of embodiment 94, wherein L^{A} is —C(O)NH-L⁴- and L⁴ is substituted or unsubstituted C₁-C₁₀ alkylene.

### Embodiment 96

The method of embodiment 95, wherein L⁴ is substituted or unsubstituted C₁-C₅ alkylene.

### Embodiment 97

The method of embodiment 96, wherein L⁴ is unsubstituted C₁-C₅ alkylene.

### Embodiment 98

The method of embodiment 97, wherein L⁴ is unsubstituted C₁-C₃ alkylene.

### Embodiment 99

The method of embodiment 09, wherein L⁴ is methylene.

### Embodiment 100

The method of embodiment 94, wherein L^{A} is -L⁴-NH— and L⁴ is substituted or unsubstituted C₁-C₁₀ alkylene.

### Embodiment 101

The method of embodiment 100, wherein L⁴ is substituted or unsubstituted C₁-C₅ alkylene.

### Embodiment 102

The method of embodiment 101, wherein L⁴ is unsubstituted C₁-C₅ alkylene.

### Embodiment 103

The method of embodiment 102, wherein L⁴ is methylene.

### Embodiment 104

The method of embodiment 94, wherein L^{A} is substituted or unsubstituted 2 to 8 membered heteroalkylene.

### Embodiment 105

The method of embodiment 94, wherein L^{A} is substituted or unsubstituted 3 to 6 membered heteroalkylene.

### Embodiment 106

The method of any one of embodiments 88-105, wherein L^{B} is independently a bond or unsubstituted C₁-C₁₀ alkylene.

### Embodiment 107

The method of embodiment 106, wherein L^{B} is a bond.

### Embodiment 108

The method of embodiment 106, wherein L^{B} is unsubstituted C₁-C₅ alkylene.

### Embodiment 109

The method of embodiment 108, wherein L^{B} is unsaturated unsubstituted C₁-C₅ alkylene.

### Embodiment 110

The method of embodiment 109, wherein L^{B} is unsaturated unsubstituted C₁-C₃ alkylene.

### Embodiment 111

The method of embodiment 110, wherein L^{B} is —C≡C—.

### Embodiment 112

The method of any one of embodiments 88-111, wherein L^{C} is a bond.

### Embodiment 113

The method of any one of embodiments 88-112, wherein ring A is substituted or unsubstituted C₅-C₆ arylene.

### Embodiment 114

The method of embodiment 113, wherein ring A is substituted or unsubstituted C₆ arylene.

### Embodiment 115

The method of embodiment 114, wherein ring A is unsubstituted phenylene.

### Embodiment 116

The method of any one of embodiments 88-115, wherein ring B is substituted or unsubstituted C₅-C₆ arylene.

### Embodiment 117

The method of embodiment 116, wherein ring B is substituted or unsubstituted C₆ arylene.

### Embodiment 118

The method of embodiment 117, wherein ring B is unsubstituted phenylene.

### Embodiment 119

The method of embodiment 87, wherein said compound has the formula:

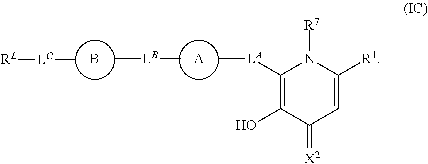

### Embodiment 120

The method of embodiment 119, wherein R¹ is R^{1A}-substituted or unsubstituted C₁-C₁₀ alkyl, wherein R^{1A} is —OH or —SO₂CH₃.

### Embodiment 121

The method of embodiment 120, wherein R¹ is R^{1A}-substituted C₁-C₅ alkyl.

### Embodiment 122

The method of embodiment 120, wherein R¹ is R^{1A}-substituted C₁-C₃ alkyl.

### Embodiment 123

The method of embodiment 120, wherein R¹ is R^{1A}-substituted C₁ alkyl.

### Embodiment 124

The method of any one of embodiments 119-122, wherein R^{L} is —C(O)NR⁵R⁶ or —OR⁵.

### Embodiment 125

The method of embodiment 124, wherein R^{L} is —C(O)NR⁵R⁶ and R⁵ and R⁶ are independently hydrogen or substituted or unsubstituted C₁-C₅ alkyl.

### Embodiment 126

The method of embodiment 125, wherein R^{L} is —C(O)NR⁵R⁶ and R⁵ and R⁶ are hydrogen.

### Embodiment 127

The method of embodiment 124, wherein R^{L} is —OR⁵ and R⁵ is substituted or unsubstituted C₁-C₅ alkyl.

### Embodiment 128

The method of embodiment 127, wherein R⁵ is unsubstituted C₁-C₅ alkyl.

### Embodiment 129

The method of embodiment 128, wherein R⁵ is methyl.

### Embodiment 130

The method of any one of embodiments 119-129, wherein L^{A} is -L⁴-NH— and L⁴ is substituted or unsubstituted C₁-C₁₀ alkylene.

### Embodiment 131

The method of embodiment 130, wherein L⁴ is unsubstituted C₁-C₅ alkylene.

### Embodiment 132

The method of embodiment 131, wherein L⁴ is methylene.

### Embodiment 133

The method of any one of embodiments 119-132, wherein ring A is substituted or unsubstituted C₅-C₆ arylene.

### Embodiment 134

The method of embodiment 133, wherein ring A is unsubstituted phenylene.

### Embodiment 135

The method of any one of embodiments 119-134, wherein ring B is substituted or unsubstituted C₅-C₆ arylene.

### Embodiment 136

The method of embodiment 135, wherein ring B is unsubstituted phenylene.

### Embodiment 137

The method of any one of embodiments 119-136, wherein R⁷ is independently hydrogen or R^{7A}-substituted or unsubstituted alkyl and R^{7A} is —OH, —COOH or unsubstituted heteroaryl.

### Embodiment 138

The method of embodiment 137, wherein R⁷ is R^{7A}-substituted C₁-C₁₀ alkyl.

### Embodiment 139

The method of embodiment 137, wherein R⁷ is R^{7A}-substituted C₁-C₅ alkyl.

### Embodiment 140

The method of embodiment 137, wherein R⁷ is R^{7A}-substituted C₃ alkyl and R^{7A} is —OH.

### Embodiment 141

The method of embodiment 137, wherein R⁷ is R^{7A}-substituted C₂ alkyl and R^{7A} is —COOH.

### Embodiment 142

The method of embodiment 137, wherein R⁷ is R^{7A}-substituted C₁ alkyl and R^{7A} is unsubstituted 5-membered heteroaryl.

### Embodiment 143

The method of embodiment 137, wherein R^{7A} is furanyl.

### Embodiment 144

The method of embodiment 87, wherein said compound has the formula:

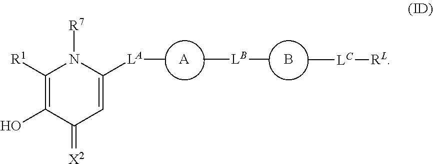

### Embodiment 145

The method of embodiment 144, wherein R¹ is hydrogen.

### Embodiment 146

The method of embodiment 145, wherein R^{L} is —CN.

### Embodiment 147

The method of any one of embodiments 144-146, wherein L^{A} is substituted or unsubstituted 2-8 membered heteroalkylene.

### Embodiment 148

The method of embodiment 147, wherein L^{A} is substituted 4 membered heteroalkylene.

### Embodiment 149

The method of any one of embodiments 144-148, wherein ring A is substituted or unsubstituted C₅-C₆ arylene.

### Embodiment 150

The method of embodiment 149, wherein ring A is unsubstituted phenylene.

### Embodiment 151

The method of any one of embodiments 144-150, wherein ring B is substituted or unsubstituted C₅-C₆ arylene.

### Embodiment 152

The method of embodiment 151, wherein ring B is unsubstituted phenylene.

### Embodiment 153

The method of any one of embodiments 144-152, wherein R⁷ is independently hydrogen or R^{7A}-substituted or unsubstituted alkyl and R^{7A} is —OH.

### Embodiment 154

The method of embodiment 153, wherein R⁷ is R^{7A}-substituted C₁-C₅ alkyl.

### Embodiment 155

The method of embodiment 154, wherein R⁷ is R^{7A}-substituted C₂ alkyl.

### Embodiment 156

The method of embodiment 84, wherein said compound has the formula:

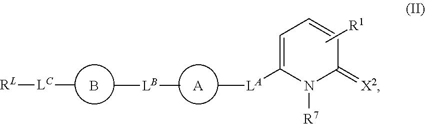

wherein R¹ is independently hydrogen, halogen, —CX^{a}₃, —CN, —SR, —SO₂Cl, —SOₙ₁R, —SOᵥ₁NR³R⁴, —NHNH₂, —ONR³R⁴, —NHC═(O)NHNH₂, —NHC═(O)NR³R⁴, —N(O)ₘ₁, —NR³R⁴, —NH—O—R³, —C(O)R³, —C(O)—OR³, —C(O)NR³R⁴, —OR³, -L⁴-SOₙ₁R³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{L} is independently hydrogen, halogen, —CX^{b}₃, —CN, —SR⁵, —SO₂Cl, —SOₙR⁵, —SOᵥ₂NR⁵R⁶, —NHNH₂, —ONR⁵R⁶, —NHC═(O)NHNH₂, —NHC═(O)NR⁵R⁶, —N(O)ₘ₂, —NR⁵R⁶, —NH—O—R⁵, —C(O)R⁵, —C(O)—OR⁵, —C(O)NR⁵R⁶, —OR⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R³, R⁴, R⁵, R⁶ and R⁷ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L^{A} is independently a bond, —S(O)—, —S(O)₂NH—, —NHS(O)₂—, —C(O)O—, —OC(O)—, —C(O)—, —C(O)NH—, —C(O)NH-L⁴-, —NH—, —NHC(O)—, —NH-L⁴-, -L⁴-NH—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L^{B} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L^{C} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L⁴ is independently —C(O)—, —C(O)—NH₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; ring A is substituted or unsubstituted cycloalkylene, substituted or unsubstituted heterocycloalkylene, substituted or unsubstituted arylene, or substituted or unsubstituted heteroarylene; ring B is substituted or unsubstituted cycloalkylene, substituted or unsubstituted heterocycloalkylene, substituted or unsubstituted arylene, or substituted or unsubstituted heteroarylene; X^{a} and X^{b} are independently —F, —Cl, —Br, or —I; n₁ and n₂ are independently an integer from 0 to 4; m₁ and m₂ are independently an integer from 1 to 2; v₁ and v₂ are independently an integer from 1 to 2; and X² is O or S.

### Embodiment 157

The method of embodiment 156, wherein R¹ is hydrogen.

### Embodiment 158

The method of embodiment 156 or 157, wherein R^{L} is independently hydrogen, —CN, substituted or unsubstituted heterocycloalkyl or substituted or unsubstituted aryl.

### Embodiment 159

The method of embodiment 158, wherein R^{L} is hydrogen.

### Embodiment 160

The method of embodiment 158, wherein R^{L} is —CN.

### Embodiment 161

The method of embodiment 158, wherein R^{L} is substituted or unsubstituted 5-6 membered heterocycloalkyl.

### Embodiment 162

The method of embodiment 161, wherein R^{L} is unsubstituted 6 membered heterocycloalkyl.

### Embodiment 163

The method of embodiment 162, wherein R^{L} is dioxanyl.

### Embodiment 164

The method of embodiment 158, wherein R^{L} is substituted or unsubstituted 5-6 membered aryl.

### Embodiment 165

The method of embodiment 164, wherein R^{L} is unsubstituted 5-6 membered aryl.

### Embodiment 166

The method of embodiment 165, wherein R^{L} is unsubstituted phenyl.

### Embodiment 167

The method of any one of embodiments 156-166, wherein L^{A} is —C(O)NH-L⁴- and L⁴ is substituted or unsubstituted C₁-C₁₀ alkylene.

### Embodiment 168

The method of embodiment 167, wherein L⁴ is substituted or unsubstituted C₁-C₅ alkylene.

### Embodiment 169

The method of embodiment 168, wherein L⁴ is unsubstituted C₁-C₃ alkylene.

### Embodiment 170

The method of embodiment 169, wherein L⁴ is methylene.

### Embodiment 171

The method of any one of embodiments 156-170, wherein L^{B} is a bond, substituted or unsubstituted alkylene or —NH—.

### Embodiment 172

The method of embodiment 171, wherein L^{B} is a bond.

### Embodiment 173

The method of embodiment 171, wherein L^{B} is substituted or unsubstituted C₁-C₅ alkylene.

### Embodiment 174

The method of embodiment 173, wherein L^{B} is unsubstituted C₁-C₅ alkylene.

### Embodiment 175

The method of embodiment 174, wherein L^{B} is unsaturated unsubstituted C₁-C₅ alkylene.

### Embodiment 176

The method of embodiment 175, wherein L^{B} is —C≡C—.

### Embodiment 177

The method of embodiment 171, wherein L^{B} is —NH—.

### Embodiment 178

The method of any one of embodiments 156-177, wherein L^{C} is a bond.

### Embodiment 179

The method of any one of embodiments 156-178, wherein ring A is substituted or unsubstituted C₅-C₆ arylene or substituted or unsubstituted 5-6 membered heteroarylene.

### Embodiment 180

The method of embodiment 179, wherein ring A is unsubstituted phenylene.

### Embodiment 181

The method of embodiment 179, wherein ring A is unsubstituted 6 membered heteroarylene.

### Embodiment 182

The method of embodiment 181, wherein ring A is unsubstituted pyridinylene.

### Embodiment 183

The method of any one of embodiments 156-182, wherein ring B is substituted or unsubstituted C₅-C₆ arylene.

### Embodiment 184

The method of embodiment 183, wherein ring B is unsubstituted phenylene.

### Embodiment 185

The method of any one of embodiments 156-184, wherein R⁷ is —OH.

### Embodiment 186

The method of embodiment 84, wherein said compound has the formula:

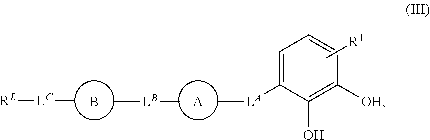

wherein R¹ is independently hydrogen, halogen, —CX^{a}₃, —CN, —SR³, —SO₂Cl, —SOₙR³, —SOᵥ₁NR³R⁴, —NHNH₂, —ONR³R⁴, —NHC═(O)NHNH₂, —NHC═(O)NR³R⁴, —N(O)ₘ₁, —NR³R⁴, —NH—O—R³, —C(O)R³, —C(O)—OR³, —C(O)NR³R⁴, —OR³, -L⁴-SOₙ₁R³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{L} is independently hydrogen, halogen, —CX^{b}₃, —CN, —SR, —SO₂Cl, —SOₙ₂R⁵, —SOᵥ₂NR⁵R⁶, —NHNH₂, —ONR⁵R⁶, —NHC═(O)NHNH₂, —NHC═(O)NR⁵R⁶, —N(O)ₘ₂, —NR⁵R⁶, —NH—O—R⁵, —C(O)R⁵, —C(O)—OR⁵, —C(O)NR⁵R⁶, —OR⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R³, R⁴, R⁵ and R⁶ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L^{A} is independently a bond, —S(O)—, —S(O)₂NH—, —NHS(O)₂—, —C(O)O—, —OC(O)—, —C(O)—, —C(O)NH—, —C(O)NH-L⁴-, —NH—, —NHC(O)—, —NH-L⁴-, -L⁴-NH—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L^{B} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L^{C} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L⁴ is independently —C(O)—, —C(O)—NH₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; ring A is substituted or unsubstituted cycloalkylene, substituted or unsubstituted heterocycloalkylene, substituted or unsubstituted arylene, or substituted or unsubstituted heteroarylene; ring B is substituted or unsubstituted cycloalkylene, substituted or unsubstituted heterocycloalkylene, substituted or unsubstituted arylene, or substituted or unsubstituted heteroarylene; X^{a} and X^{b} are independently —F, —Cl, —Br, or —I; n₁ and n₂ are independently an integer from 0 to 4; m₁ and m₂ are independently an integer from 1 to 2; and v₁ and v₂ are independently an integer from 1 to 2.

### Embodiment 187

The method of embodiment 186, wherein R¹ is hydrogen.

### Embodiment 188

The method of embodiment 186, wherein R^{L} is —CN.

### Embodiment 189

The method of any one of embodiments 186-188, wherein L^{A} is —C(O)NH-L⁴- and L⁴ is substituted or unsubstituted C₁-C₁₀ alkylene.

### Embodiment 190

The method of embodiment 189, wherein L⁴ is substituted or unsubstituted C₁-C₅ alkylene.

### Embodiment 191

The method of embodiment 189, wherein L⁴ is unsubstituted C₁-C₃ alkylene.

### Embodiment 192

The method of embodiment 189, wherein L⁴ is methylene.

### Embodiment 193

The method of any one of embodiments 186-192, wherein L^{B} is a bond.

### Embodiment 194

The method of any one of embodiments 186-193, wherein L^{C} is a bond.

### Embodiment 195

The method of any one of embodiments 186-194, wherein ring A is substituted or unsubstituted C₅-C₆ arylene.

### Embodiment 196

The method of embodiment 195, wherein ring A is unsubstituted phenylene.

### Embodiment 197

The method of any one of embodiments 186-196, wherein ring B is substituted or unsubstituted C₅-C₆ arylene.

### Embodiment 198

The method of embodiment 197, wherein ring B is unsubstituted phenylene.

### Embodiment 199

The method of embodiment 84, wherein said compound has the formula:

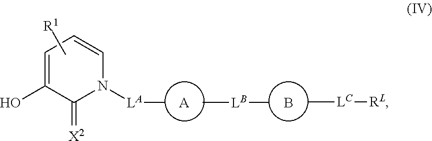

wherein R¹ is independently hydrogen, halogen, —CX^{a}₃, —CN, —SR³, —SO₂Cl, —SOₙ₁R³, —SOᵥ₁NR³R⁴, —NHNH₂, —ONR³R⁴, —NHC═(O)NHNH₂, —NHC═(O)NR³R⁴, —N(O)ₘ₁, —NR³R⁴, —NH—O—R³, —C(O)R³, —C(O)—OR³, —C(O)NR³R⁴, —OR³, -L⁴-SOₙ₁R³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{L} is independently hydrogen, halogen, —CX^{b}₃, —CN, —SR, —SO₂Cl, —SO₂R, —SOₙ₂NR⁵R⁶, —NHNH₂, —ONR⁵R⁶, —NHC═(O)NHNH₂, —NHC═(O)NR⁵R⁶, —N(O)ₙ₂, —NR⁵R⁶, —NH—O—R⁵, —C(O)R⁵, —C(O)—OR⁵, —C(O)NR⁵R⁶, —OR⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R³, R⁴, R⁵ and R⁶ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L^{A} is independently a bond, —S(O)—, —S(O)₂NH—, —NHS(O)₂—, —C(O)O—, —OC(O)—, —C(O)—, —C(O)NH—, —C(O)NH-L⁴-, —NH—, —NHC(O)—, —NH-L⁴-, -L⁴-NH—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L^{B} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L^{C} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L⁴ is independently —C(O)—, —C(O)—NH₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; ring A is substituted or unsubstituted cycloalkylene, substituted or unsubstituted heterocycloalkylene, substituted or unsubstituted arylene, or substituted or unsubstituted heteroarylene; ring B is substituted or unsubstituted cycloalkylene, substituted or unsubstituted heterocycloalkylene, substituted or unsubstituted arylene, or substituted or unsubstituted heteroarylene; X^{a} and X^{b} are independently —F, —Cl, —Br, or —I; n₁ and n₂ are independently an integer from 0 to 4; m₁ and m₂ are independently an integer from 1 to 2; v₁ and v₂ are independently an integer from 1 to 2; and X² is O or S.

### Embodiment 200

The method of embodiment 100, wherein R¹ is hydrogen.

### Embodiment 201

The method of embodiment 100 or 200, wherein R^{L} is hydrogen.

### Embodiment 202

The method of any one of embodiments 199-201, wherein L^{A} is substituted or unsubstituted hetero alkylene.

### Embodiment 203

The method of embodiment 202, wherein L^{A} is substituted 2-8 membered heteroalkylene.

### Embodiment 204

The method of embodiment 203, wherein L^{A} is substituted 4 membered heteroalkylene.

### Embodiment 205

The method of any one of embodiments 199-204, wherein L^{B} is a bond.

### Embodiment 206

The method of any one of embodiments 199-205, wherein L is a bond.

### Embodiment 207

The method of any one of embodiments 100-206, wherein ring A is substituted or unsubstituted C₅-C₆ arylene.

### Embodiment 208

The method of embodiment 207, wherein ring A is unsubstituted phenylene.

### Embodiment 209

The method of any one of embodiments 199-208, wherein ring B is substituted or unsubstituted C₅-C₆ arylene.

### Embodiment 210

The method of embodiment 209, wherein ring B is unsubstituted phenylene.

### Embodiment 211

The method of any one of embodiments 84-210, wherein said disease is an infectious disease.

### Embodiment 212

The method of embodiment 211, wherein said disease is a bacterial disease.

### Embodiment 213

The method of embodiment 212, wherein said disease is caused by gram-negative bacteria.

### Embodiment 214

A compound of formula:

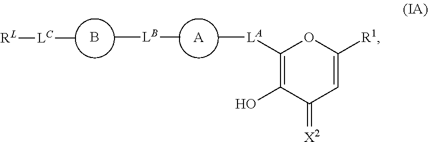

wherein R¹ is independently hydrogen, halogen, —CX^{a}₃, —CN, —SR³, —SO₂Cl, —SOₙ₁R³, —SOᵥ₁NR³R⁴, —NHNH₂, —ONR³R⁴, —NHC═(O)NHNH₂, —NHC═(O)NR³R⁴, —N(O)ₘ₁, —NR³R⁴, —NH—O—R³, —C(O)R³, —C(O)—OR³, —C(O)NR³R⁴, —OR³, -L⁴-SOₙ₁R³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{L} is independently hydrogen, halogen, —CX^{b}₃, —CN, —SR⁵, —SO₂Cl, —SOₙ₂R⁵, —SOᵥ₂NR⁵R⁶, —NHNH₂, —ONR⁵R⁶, —NHC═(O)NHNH₂, —NHC═(O)NR⁵R⁶, —N(O)ₘ₂, —NR⁵R⁶, —NH—O—R⁵, —C(O)R⁵, —C(O)—OR⁵, —C(O)NR⁵R⁶, —OR⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R³, R⁴, R⁵, R⁶ and R⁷ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L^{A} is independently a bond, —S(O)—, —S(O)₂NH—, —NHS(O)₂—, —C(O)O—, —OC(O)—, —C(O)—, -L⁴-C(O)NH—, —NH—, —NHC(O)—, —NH-L⁴-, -L⁴-NH—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L^{B} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L^{C} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L⁴ is independently —C(O)—, —C(O)—NH—, —NH—C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; ring A is substituted or unsubstituted cycloalkylene, substituted or unsubstituted heterocycloalkylene, substituted or unsubstituted arylene, or substituted or unsubstituted heteroarylene; ring B is substituted or unsubstituted cycloalkylene, substituted or unsubstituted heterocycloalkylene, substituted or unsubstituted arylene, or substituted or unsubstituted heteroarylene; X¹ is C, N, O, NR⁷ or S; X² is independently O or S; X^{a} and X^{b} are independently F, Cl, Br, or I; n₁ and n₂ are independently an integer from 0 to 4; m₁ and m₂ are independently an integer from 1 to 2; and v₁ and v₂ are independently an integer from 1 to 2; wherein if L^{A} is -L⁴-C(O)NH—, then L⁴ is not a bond.

### Additional Embodiments

Additional embodiments include embodiments P1-P11 following:

### Embodiment P1

A compound with structure of Formula (PII):

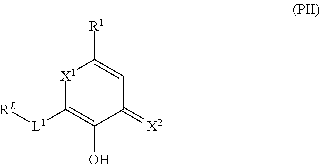

wherein R¹ is hydrogen, —CN, —SH, —OH, —COOH, —NH₂, —CONH₂, nitro, —CF₃, —CCl₃, substituted or unsubstituted alkyl, or substituted or unsubstituted heteroalkyl; L¹ is substituted or unsubstituted alkylene, or substituted or unsubstituted heteroalkylene; R^{L} is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; X¹ is C, N, O or S; and X² is O or S.

### Embodiment P2

The compound of embodiment Error! Reference source not found, wherein R¹ is hydrogen, methyl, hydroxymethyl, —C(O)OCH₃, —CH₂S(O)₂CH₃, or —CH₂S(O)₂-phenyl.

### Embodiment P3

The compound of embodiment P1, wherein L¹ is —NH—CH₂—.

### Embodiment P4

The compound of embodiment P1, wherein X¹ is C.

### Embodiment P5

The compound of embodiment P1, wherein X¹ is N.

### Embodiment P6

The compound of embodiment P1, wherein X¹ is O.

### Embodiment P7

The compound of embodiment P1, wherein X¹ is S.

### Embodiment P8

The compound of embodiment P1, wherein X² is O.

### Embodiment P9

The compound of embodiment P1, wherein X² is S.

### Embodiment P10

A pharmaceutical composition comprising a pharmaceutically acceptable excipient and a compound according to embodiment P1.

### Embodiment P11

A method for treating an infection, said method comprising administering to a subject in need a therapeutically effective amount of a compound according to embodiment P1.

1. A compound of formula: 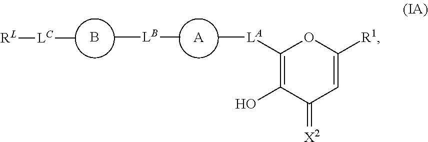
wherein
R¹ is independently hydrogen, halogen, —CX^{a}₃, —CN, —SR³, —SO₂Cl, —SOₙ₁R³, —SOᵥ₁NR³R⁴, —NHNH₂, —ONR³R⁴, —NHC═(O)NHNH₂, —NHC═(O)NR³R⁴, —N(O)ₘ₁, —NR³R⁴, —NH—O—R³, —C(O)R³, —C(O)—OR³, —C(O)NR³R⁴, —OR³, -L⁴-SOₙ₁R³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{L} is independently hydrogen, halogen, —CX^{b}₃, —CN, —SR⁵, —SO₂Cl, —SOₙ₂R⁵, —SOᵥ₂NR⁵R⁶, —NHNH₂, —ONR⁵R⁶, —NHC═(O)NHNH₂, —NHC═(O)NR⁵R⁶, —N(O)ₘ₂, —NR⁵R⁶, —NH—O—R⁵, —C(O)R⁵, —C(O)—OR⁵, —C(O)NR⁵R⁶, —OR⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³, R⁴, R⁵, R⁶ and R⁷ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{A} is independently a bond, —S(O)—, —S(O)₂NH—, —NHS(O)₂—, —C(O)O—, —OC(O)—, —C(O)—, -L⁴-C(O)NH—, —NH—, —NH-L⁴-, -L⁴-NH—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{B} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{C} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is independently —C(O)—, —C(O)—NH—, —NH—C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
ring A is substituted or unsubstituted cycloalkylene, substituted or unsubstituted heterocycloalkylene, substituted or unsubstituted arylene, or substituted or unsubstituted heteroarylene;
ring B is substituted or unsubstituted cycloalkylene, substituted or unsubstituted heterocycloalkylene, substituted or unsubstituted arylene, or substituted or unsubstituted heteroarylene;
X¹ is C, N, O, NR⁷ or S;
X² is independently O or S;
X^{a} and X^{b} are independently F, Cl, Br, or I;
n₁ and n₂ are independently an integer from 0 to 4;
m₁ and m₂ are independently an integer from 1 to 2; and
v₁ and v₂ are independently an integer from 1 to 2;
wherein if L^{A} is -L⁴-C(O)NH—, then L⁴ is not a bond.

2. The compound of claim 1, wherein R¹ is independently R^{1A-}substituted or unsubstituted alkyl, R^{1A}-substituted or unsubstituted heteroalkyl, R^{1A}-substituted or unsubstituted cycloalkyl, R^{1A}-substituted or unsubstituted heterocycloalkyl, R^{1A}-substituted or unsubstituted aryl, or R^{1A}-substituted or unsubstituted heteroaryl;
R^{1A} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1B}-substituted or unsubstituted alkyl, R^{1B}-substituted or unsubstituted heteroalkyl, R^{1B}-substituted or unsubstituted cycloalkyl, R^{1B}-substituted or unsubstituted heterocycloalkyl, R^{1B}-substituted or unsubstituted aryl, or R^{1B}-substituted or unsubstituted heteroaryl;
R^{1B} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R^{1C}-substituted or unsubstituted alkyl, R^{1C}-substituted or unsubstituted heteroalkyl, R^{1C}-substituted or unsubstituted cycloalkyl, R^{1C}-substituted or unsubstituted heterocycloalkyl, R^{1C}-substituted or unsubstituted aryl, or R^{1C}-substituted or unsubstituted heteroaryl;
R^{1C} is independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl.

3. (canceled)

4. (canceled)

5. (canceled)

6. The compound of claim 1, wherein R¹ is -L⁴-SOₙ₁R³, R³ is substituted or unsubstituted alkyl or substituted or unsubstituted aryl, and L⁴ is substituted or unsubstituted alkylene.

7. The compound of claim 1, wherein R³ is unsubstituted C₁-C₅ alkyl or C₅-C₆ aryl.

8. (canceled)

9. (canceled)

10. (canceled)

11. The compound of claim 1, wherein L⁴ is substituted or unsubstituted C₁-C₁₀ alkylene.

12. (canceled)

13. (canceled)

14. The compound of claim 1, wherein R¹ is substituted or unsubstituted C₁-C₁₀ alkyl, R^{1A}-substituted C₁-C₅ alkyl or R^{1A}-substituted C₁ alkyl, wherein R^{1A} is —OH.

15. (canceled)

16. (canceled)

17. (canceled)

18. (canceled)

19. The compound of claim 1, wherein R^{L} is independently hydrogen, halogen, —OR⁵, —C(O)—OR⁵, —NR⁵R⁶, —C(O)NR⁵R⁶, substituted or unsubstituted heterocycloalkyl or substituted or unsubstituted aryl.

20. (canceled)

21. (canceled)

22. (canceled)

23. (canceled)

24. (canceled)

25. The compound of claim 1, wherein R^{L} is —F, —OR⁵, —C(O)—OR⁵, —C(O)NR⁵R⁶ or —NR⁵R⁶ and R⁵ and R⁶ are independently hydrogen or substituted or unsubstituted C₁-C₁₀ alkyl.

26. (canceled)

27. (canceled)

28. (canceled)

29. The compound of claim 1, wherein R^{L} is substituted or unsubstituted 5-6 membered heterocycloalkyl or substituted or unsubstituted C₅-C₆ aryl.

30. (canceled)

31. (canceled)

32. (canceled)

33. The compound of claim 1, wherein L^{A} is -L⁴-NH— and L⁴ is substituted or unsubstituted C₁-C₁₀ alkylene.

34. (canceled)

35. (canceled)

36. The compound of claim 1, wherein L^{B} is independently a bond or unsubstituted C₁-C₁₀ alkylene or an unsaturated unsubstituted C₁-C₅ alkylene.

37. (canceled)

38. (canceled)

39. (canceled)

40. The compound of claim 1, wherein ring A is substituted or unsubstituted C₅-C₆ arylene.

41. (canceled)

42. (canceled)

43. (canceled)

44. The compound of claim 1, wherein said compound has the formula:

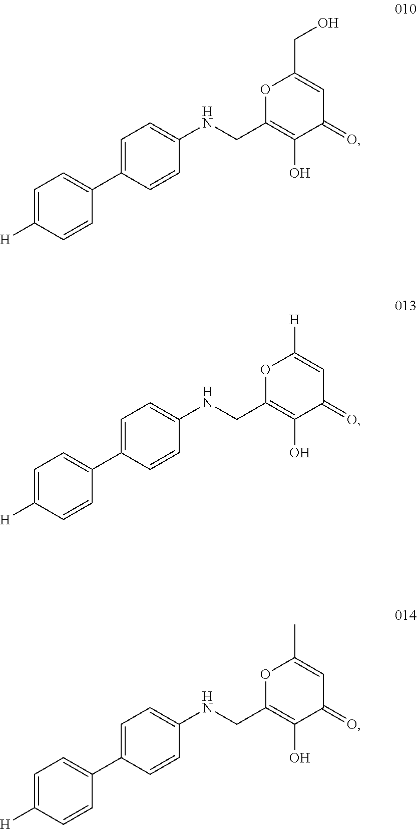 [SMILES: O=c1cc(CO)oc(CNc2ccc(-c3ccccc3)cc2)c1O ; O=c1ccoc(CNc2ccc(-c3ccccc3)cc2)c1O ; Cc1cc(=O)c(O)c(CNc2ccc(-c3ccccc3)cc2)o1]

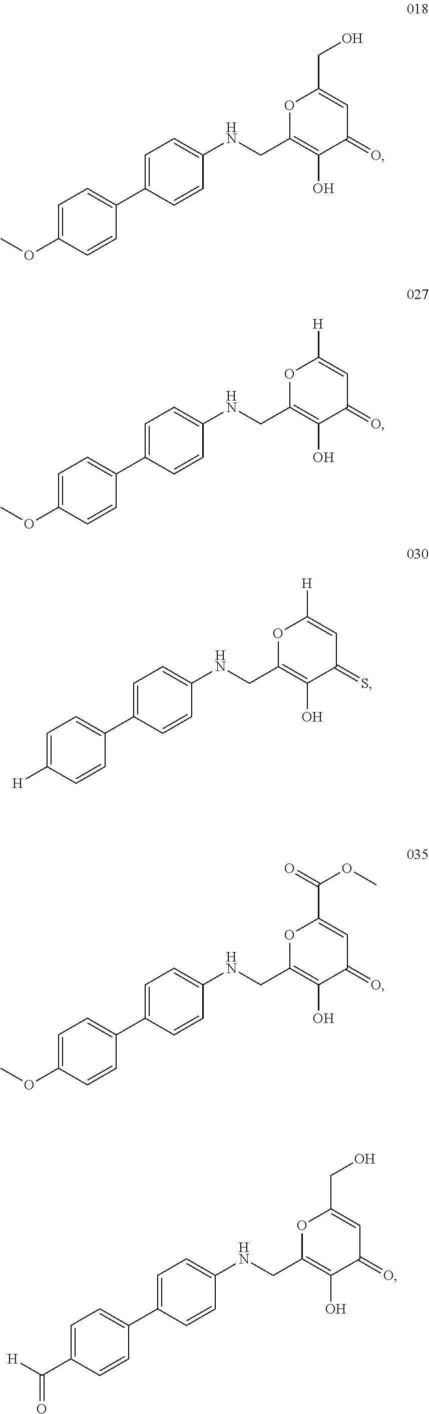

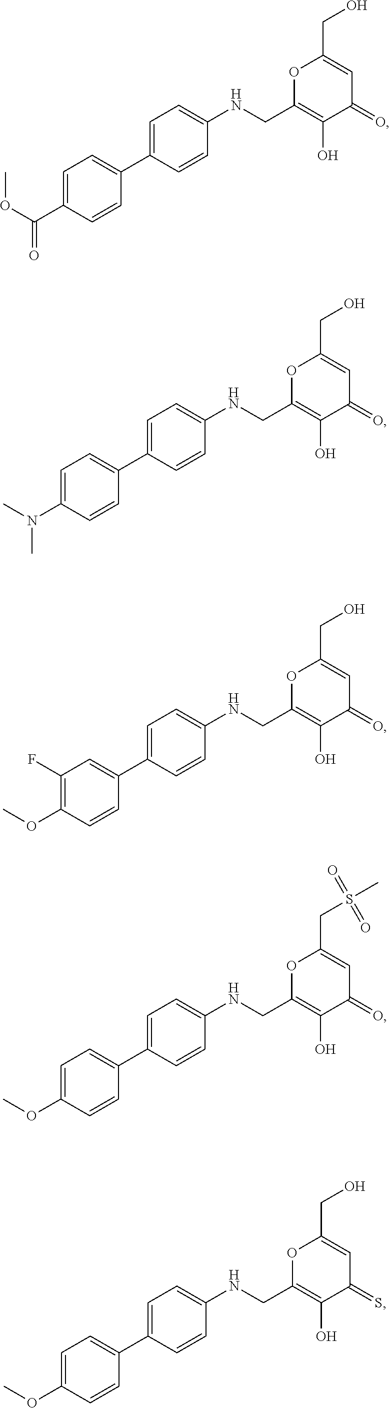

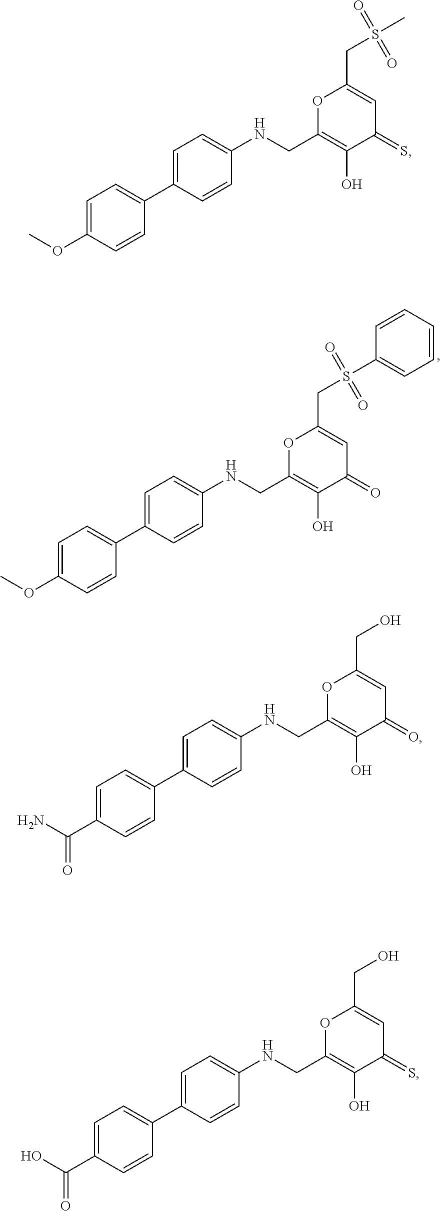

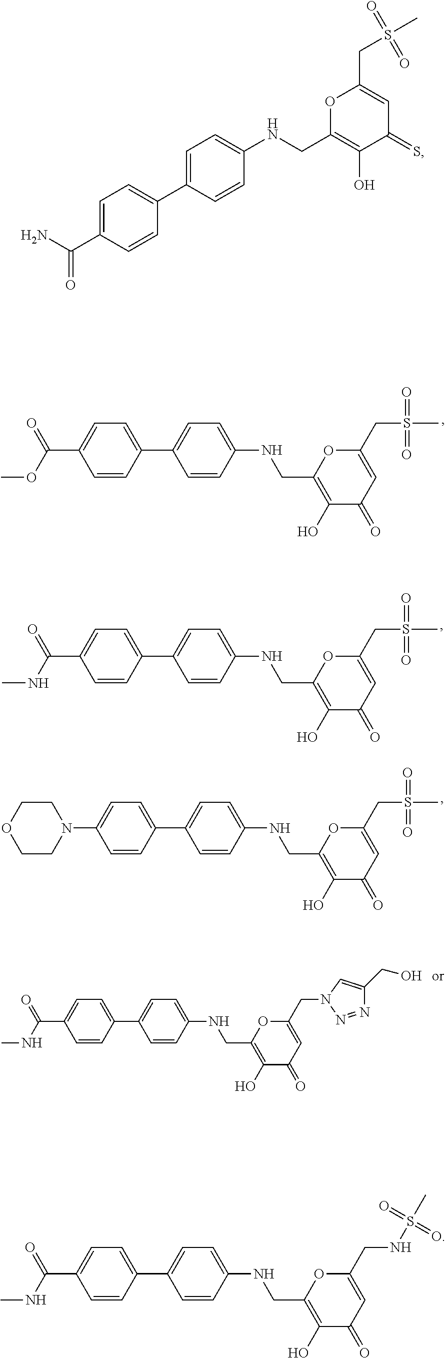

45. A pharmaceutical composition comprising a pharmaceutically acceptable excipient and a compound of claim 1.

46. (canceled)

47. (canceled)

48. (canceled)

49. (canceled)

50. A method of treating a UDP-3-O—(R-3-hydroxymyristoyl)-N-acetyl lucosamine deacetylase (LpxC)-mediated disease in a patient in need of such treatment, said method comprising administering a therapeutically effective amount of a compound of formula: 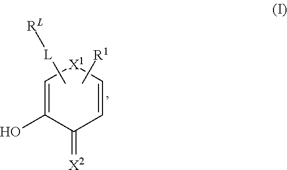
wherein
R¹ is independently hydrogen, halogen, —CX^{a}₃, —CN, —SR³, —SO₂Cl, —SOₙ₁R³, —SOᵥ₁NR³R⁴, —NHNH₂, —ONR³R⁴, —NHC═(O)NHNH₂, —NHC═(O)NR³R⁴, —N(O)ₘ₁, —NR³R⁴, —NH—O—R³, —C(O)R³, —C(O)—OR³, —C(O)NR³R⁴, —OR³, -L⁴-SOₙ₁R³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{L} is independently hydrogen, halogen, —CX^{b}₃, —CN, —SR⁵, —SO₂Cl, —SOₙ₂R⁵, —SOᵥ₂NR⁵R⁶, —NHNH₂, —ONR⁵R⁶, —NHC═(O)NHNH₂, —NHC═(O)NR⁵R⁶, —N(O)ₘ₂, —NR⁵R⁶, —NH—O—R⁵, —C(O)R⁵, —C(O)—OR⁵, —C(O)NR⁵R⁶, —OR⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³, R⁴, R⁵, R⁶ and R⁷ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L is independently a bond, —S(O)—, —S(O)₂NH—, —NHS(O)₂—, —C(O)O—, —OC(O)—, —C(O)—, —C(O)NH—, —C(O)NH-L⁵-, -L⁵-C(O)NH—, —NH—, —NHC(O)—, —NH-L⁵-, -L⁵-NH—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁵ is independently —C(O)—, —C(O)—NH₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
X^{a} and X^{b} are independently —F, —Cl, —Br, or —I;
n₁ and n₂ are independently an integer from 0 to 4;
m₁ and m₂ are independently an integer from 1 to 2;
v₁ and v₂ are independently an integer from 1 to 2;
X¹ is C, N, O, NR⁷ or S; and
X² is O or S.

51. The method of claim 50, wherein L is 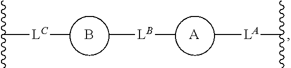
wherein
L^{A} is independently a bond, —S(O)—, —S(O)₂NH—, —NHS(O)₂—, —C(O)O—, —OC(O)—, —C(O)—, —C(O)NH—, —C(O)NH-L⁴-, —NH—, —NHC(O)—, —NH-L⁴-, -L⁴-NH—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{B} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{C} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is independently —C(O)—, —C(O)—NH₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
ring A is substituted or unsubstituted cycloalkylene, substituted or unsubstituted heterocycloalkylene, substituted or unsubstituted arylene, or substituted or unsubstituted heteroarylene; and
ring B is substituted or unsubstituted cycloalkylene, substituted or unsubstituted heterocycloalkylene, substituted or unsubstituted arylene, or substituted or unsubstituted heteroarylene.

52. A method of treating a UDP-3-O—(R-3-hydroxymyristoyl)-N-acetyl lucosamine deacetylase (LpxC)-mediated disease in a patient in need of such treatment, said method comprising administering a therapeutically effective amount of a compound of formula: 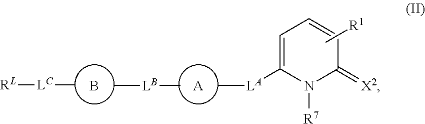
wherein
R¹ is independently hydrogen, halogen, —CX^{a}₃, —CN, —SR³, —SO₂Cl, —SOₙ₁R³, —SOᵥ₁NR³R⁴, —NHNH₂, —ONR³R⁴, —NHC═(O)NHNH₂, —NHC═(O)NR³R⁴, —N(O)ₘ₁, —NR³R⁴, —NH—O—R³, —C(O)R³, —C(O)—OR³, —C(O)NR³R⁴, —OR³, -L⁴-SOₙ₁R³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{L} is independently hydrogen, halogen, —CX^{b}₃, —CN, —SR⁵, —SO₂Cl, —SOₙ₂R⁵, —SOᵥ₂NR⁵R⁶, —NHNH₂, —ONR⁵R⁶, —NHC═(O)NHNH₂, —NHC═(O)NR⁵R⁶, —N(O)ₘ₂, —NR⁵R⁶, —NH—O—R⁵, —C(O)R⁵, —C(O)—OR⁵, —C(O)NR⁵R⁶, —OR⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³, R⁴, R⁵, R⁶ and R⁷ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{A} is independently a bond, —S(O)—, —S(O)₂NH—, —NHS(O)₂—, —C(O)O—, —OC(O)—, —C(O)—, —C(O)NH—, —C(O)NH-L⁴-, —NH—, —NHC(O)—, —NH-L⁴-, -L⁴-NH—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{B} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{C} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is independently —C(O)—, —C(O)—NH₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
ring A is substituted or unsubstituted cycloalkylene, substituted or unsubstituted heterocycloalkylene, substituted or unsubstituted arylene, or substituted or unsubstituted heteroarylene;
ring B is substituted or unsubstituted cycloalkylene, substituted or unsubstituted heterocycloalkylene, substituted or unsubstituted arylene, or substituted or unsubstituted heteroarylene;
X^{a} and X^{b} are independently —F, —Cl, —Br, or —I;
n₁ and n₂ are independently an integer from 0 to 4;
m₁ and m₂ are independently an integer from 1 to 2;
v₁ and v₂ are independently an integer from 1 to 2; and
X² is O or S.

53. A method of treating a UDP-3-O—(R-3-hydroxymyristoyl)-N-acetylglucosamine deacetylase (LpxC)-mediated disease in a patient in need of such treatment, said method comprising administering a therapeutically effective amount of a compound of formula: 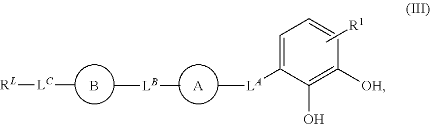
wherein
R¹ is independently hydrogen, halogen, —CX^{a}₃, —CN, —SR³, —SO₂Cl, —SOₙ₁R³, —SOᵥ₁NR³R⁴, —NHNH₂, —ONR³R⁴, —NHC═(O)NHNH₂, —NHC═(O)NR³R⁴, —N(O)ₘ₁, —NR³R⁴, —NH—O—R³, —C(O)R³, —C(O)—OR³, —C(O)NR³R⁴, —OR³, -L⁴-SOₙ₁R³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{L} is independently hydrogen, halogen, —CX^{b}₃, —CN, —SR, —SO₂Cl, —SOₙ₂R⁵, —SOᵥ₂NR⁵R⁶, —NHNH₂, —ONR⁵R⁶, —NHC═(O)NHNH₂, —NHC═(O)NR⁵R⁶, —N(O)ₘ₂, —NR⁵R⁶, —NH—O—R⁵, —C(O)R⁵, —C(O)—OR⁵, —C(O)NR⁵R⁶, —OR⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³, R⁴, R⁵ and R⁶ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{A} is independently a bond, —S(O)—, —S(O)₂NH—, —NHS(O)₂—, —C(O)O—, —OC(O)—, —C(O)—, —C(O)NH—, —C(O)NH-L⁴-, —NH—, —NHC(O)—, —NH-L⁴-, -L⁴-NH—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{B} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{C} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is independently —C(O)—, —C(O)—NH₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
ring A is substituted or unsubstituted cycloalkylene, substituted or unsubstituted heterocycloalkylene, substituted or unsubstituted arylene, or substituted or unsubstituted heteroarylene;
ring B is substituted or unsubstituted cycloalkylene, substituted or unsubstituted heterocycloalkylene, substituted or unsubstituted arylene, or substituted or unsubstituted heteroarylene;
X^{a} and X^{b} are independently —F, —Cl, —Br, or —I;
n₁ and n₂ are independently an integer from 0 to 4;
m₁ and m₂ are independently an integer from 1 to 2; and
v₁ and v₂ are independently an integer from 1 to 2.

54. A method of treating a UDP-3-O—(R-3-hydroxymyristoyl)-N-acetyl lucosamine deacetylase (LpxC)-mediated disease in a patient in need of such treatment, said method comprising administering a therapeutically effective amount of a compound of formula: 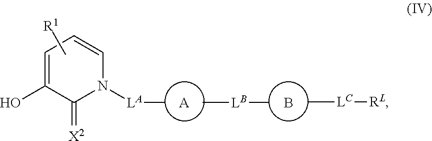
wherein
R¹ is independently hydrogen, halogen, —CX^{a}₃, —CN, —SR³, —SO₂Cl, —SOₙR³, —SOᵥ₁NR³R⁴, —NHNH₂, —ONR³R⁴, —NHC═(O)NHNH₂, —NHC═(O)NR³R⁴, —N(O)ₘ₁, —NR³R⁴, —NH—O—R³, —C(O)R³, —C(O)—OR³, —C(O)NR³R⁴, —OR³, -L⁴-SOₙ₁R³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{L} is independently hydrogen, halogen, —CX^{b}₃, —CN, —SR⁵, —SO₂Cl, —SOₙ₂R⁵, —SOᵥ₂NR⁵R⁶, —NHNH₂, —ONR⁵R⁶, —NHC═(O)NHNH₂, —NHC═(O)NR⁵R⁶, —N(O)ₘ₂, —NR⁵R⁶, —NH—O—R⁵, —C(O)R⁵, —C(O)—OR⁵, —C(O)NR⁵R⁶, —OR⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³, R⁴, R⁵ and R⁶ are independently hydrogen, halogen, ═O, ═S, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{A} is independently a bond, —S(O)—, —S(O)₂NH—, —NHS(O)₂—, —C(O)O—, —OC(O)—, —C(O)—, —C(O)NH—, —C(O)NH-L⁴-, —NH—, —NHC(O)—, —NH-L⁴-, -L⁴-NH—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{B} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{C} is independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁴ is independently —C(O)—, —C(O)—NH₂—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
ring A is substituted or unsubstituted cycloalkylene, substituted or unsubstituted heterocycloalkylene, substituted or unsubstituted arylene, or substituted or unsubstituted heteroarylene;
ring B is substituted or unsubstituted cycloalkylene, substituted or unsubstituted heterocycloalkylene, substituted or unsubstituted arylene, or substituted or unsubstituted heteroarylene;
X^{a} and X^{b} are independently —F, —Cl, —Br, or —I;
n₁ and n₂ are independently an integer from 0 to 4;
m₁ and m₂ are independently an integer from 1 to 2;
v₁ and v₂ are independently an integer from 1 to 2; and
X² is O or S.

55. The method of claim 50, 52, 53 or 54, wherein said disease is an infectious disease, a bacterial disease or said disease is caused by gram-negative bacteria.

56.-57. (canceled)
